(19) United States
(12) Patent Application Publication
(10) Pub. No.: US 2013/0143752 A1
(43) Pub. Date: Jun. 6, 2013

(54) GENE BIOMARKERS OF LUNG FUNCTION

(71) Applicants: Jeffery S. Edmiston, Mechanicsville, VA (US); Barbara K. Zedler, Richmond, VA (US); Edward Lenn Murrelle, Midlothian, VA (US); Mark Leppert, Salt Lake City, UT (US); Kellie J. Archer, Richmond, VA (US); Mariano J. Scian, Charlottesville, VA (US)

(72) Inventors: Jeffery S. Edmiston, Mechanicsville, VA (US); Barbara K. Zedler, Richmond, VA (US); Edward Lenn Murrelle, Midlothian, VA (US); Mark Leppert, Salt Lake City, UT (US); Kellie J. Archer, Richmond, VA (US); Mariano J. Scian, Charlottesville, VA (US)

(21) Appl. No.: 13/541,349

(22) Filed: Jul. 3, 2012

Related U.S. Application Data
(63) Continuation of application No. PCT/US2011/000016, filed on Jan. 4, 2011.
(60) Provisional application No. 61/292,154, filed on Jan. 4, 2010.

(51) Int. Cl. C12Q 1/68

(52) U.S. Cl. CPC C12Q 1/6876
USPC 506/9; 506/17; 506/39

(57) ABSTRACT

Described herein are a group of 1,013 genes and 1 phenotypic variable are identified as candidate predictors that differentiated smokers (current or former) with or without COPD. The full predictor set can be reduced to a nine-gene classifier (IL6R, CCR2, PPP2CB, RASSF2, WTAP, DNTTIP2, GDAP1, LIPE, and RPL14) with similar performance. Also described herein is the use of the full predictor set and the reduced nine gene set in methods of diagnosing lung disease or an increased risk of developing lung disease, such as COPD, in a subject. Also described herein is the use of the full predictor set and the reduced nine gene set in methods of providing a prognosis for a subject with lung disease, such as COPD.

This application claims the benefit of U.S. Provisional Application Ser. No. 61/292,154, filed Jan. 4, 2010, entitled “GENE BIOMARKERS OF LUNG FUNCTION” the entirety of which is hereby incorporated by reference.

### BACKGROUND

Lung diseases impair lung function and, according to the American Lung Association, are the third primary cause of death in America, accounting for one in six deaths. The main categories of lung disease include airway diseases, lung tissue diseases and pulmonary circulation diseases as well as combinations of the above. Examples of diseases affecting lung function include asthma, chronic obstructive pulmonary disease (COPD), lung cancer, alpha-1 antitrypsin deficiency, respiratory distress syndrome, chronic bronchitis, chronic systemic inflammation, and inflammatory respiratory disease among others.

COPD is the fourth leading cause of morbidity and mortality in the United States and is expected to rank third as the cause of death, worldwide, by 2020 (Mannino and Braman, 2007, Proceedings of the American Thoracic Society 4:502-506). Cigarette smoking is widely recognized as a primary causative factor of COPD and accounts for approximately 80-99% of all cases in the United States. COPD is characterized by chronic airflow limitation, measured spirometrically by the ratio of the forced expiratory volume in one second (FEV₁) to the forced vital capacity (FVC), and associated with an abnormal inflammatory response of the lung to noxious particles or gases. The operational diagnosis of lung diseases such as COPD has traditionally been made by spirometry, as a ratio of FEV₁ to FVC below 70% (Rabe et al., 2007, American Journal of Respiratory and Critical Care Medicine 176:532-555).

Prior diagnostic methods of COPD and other lung diseases employ diagnostic tests which rely on the presumed correlation of decreased pulmonary function with lung disease such as COPD, asthma, fibrosis, emphysema and others. While lung function tests can provide a general assessment of the functional status of a subject's lungs, the tests do not distinguish between the different types of lung diseases that may be present. For example, certain diseases such as asthma cannot be confirmed based on functional tests alone. In addition, it is only when a measurable change in lung function exists that such tests aid in the diagnosis of a lung disease.

Studies of mechanisms underlying lung diseases are hampered by the procedures required to obtain samples of disease tissue. In particular, studies investigating differential gene expression associated with lung disease have been hindered by the invasiveness of procedures used to obtain sample tissue from diseased and normal subjects. Methods which provide an accurate diagnosis of lung disease prior to development measurable changes in lung function using less invasive tissue sampling techniques would be desirable.

### SUMMARY

Novel gene biomarkers of lung function are provided. In one aspect, the gene biomarkers are identified using comparisons of gene expression profiles in subjects with a lung disease and in subjects not having the disease. In another aspect, the profiles are obtained using a method comprising high-throughput analysis. Compositions and devices comprising the novel gene biomarkers are also provided. The gene biomarkers also are useful as prognostic or diagnostic indicators of lung disease or as an indicator of a subject's risk of developing lung disease. In an additional aspect, the lung disease is COPD.

In one embodiment, gene biomarkers of lung function comprise one, two, three, four, five, six, seven, eight or more genes selected from the group of genes set forth in Supplementary Table II. In another embodiment a gene biomarker of lung function is selected from a nucleic acid molecule (polynucleotide) having a nucleotide sequence of a gene set forth in Supplementary Table II, or a nucleic acid molecule (polynucleotide) having a sequence with 70-99% identity to the nucleic acid sequence of a gene set forth in Supplementary Table II, or a fragment thereof. In another embodiment a gene biomarker of lung function is selected from a nucleic acid molecule comprising a nucleotide sequence of a gene selected from IL6R, CCR2, PPP2CB, RASSF2, WTAP, DNTTIP2, GDAP1, LIPE, and RPL14, or a nucleic acid molecule comprising a sequence with 70-99% identity to the nucleic acid sequences of a genes selected from IL6R, CCR2, PPP2CB, RASSF2, WTAP, DNTTIP2, GDAP1, LIPE, and RPL14, or a fragment thereof. It is understood that such nucleic acid molecules and fragments thereof include the sequence of the coding strand or the non-coding strand of the gene, or a fragment thereof unless stated otherwise. It is also understood that such nucleic acid molecules and fragments may comprise the sequences found in either the exons and/or introns of the genes set forth in Supplementary Table II unless stated otherwise.

The present disclosure provides for a composition comprising nucleic acids having the nucleotide sequence of a gene biomarker of lung function. In one embodiment the disclosure provides for compositions comprising two nucleic acid molecules wherein the first nucleic acid molecule comprises a first nucleotide sequence and the second nucleic acid molecule comprises a second nucleotide sequence, wherein the first nucleotide sequence differs from the second nucleotide sequence and the first and second nucleotide sequences are selected independently from the group consisting of the nucleotide sequences of the genes set forth in Supplementary Table II, or a sequence having 70-99% identity to the nucleotide sequences of the genes set forth in Supplementary Table II, or a fragment thereof. In other embodiments the disclosure provides for compositions further comprising a third, forth, fifth, sixth, seventh, eighth and/or ninth nucleic acid molecules.

Also provided is a device comprising a plurality of locations (e.g., a chip or slide bearing an array), wherein 2, 3, 4, 5, 6, 7, 8 or more of said locations each comprise a different nucleic acid molecule comprising a nucleotide sequence of a gene set forth in Supplementary Table H, or a sequence having 70-99% identity to the nucleotide sequences of a gene as set forth in Supplementary Table II, or a fragment thereof (e.g., a fragment of the protein coding exon regions).

In one embodiment, the disclosure provides a method of identifying a gene biomarker associated with lung disease by employing statistical analysis of nucleic acid sequences differentially expressed in subjects having lung disease as compared to control subjects without the disease. In one aspect, the gene biomarkers of lung disease are identified as the group of genes set forth in Supplementary Table II. In another embodiment, the gene biomarkers of lung function are identified as one or more genes (or nucleic acids encoding those genes) selected from: IL6R, CCR2, PPP2CB, RASSF2, WTAP, DNTTIP2, GDAP1, LIPE, and RPL14. Exemplary lung diseases include, for example, asthma, chronic obstructive pulmonary disease, lung cancer, alpha-1 antitrypsin deficiency, respiratory distress syndrome, chronic bronchitis, chronic systemic inflammation, and inflammatory respiratory disease, among others. In one embodiment, lung diseases or disorders may exclude cancers and/or tumors of the lungs, airways, or of other respiratory tissues. In another embodiment lung diseases may exclude one or more of asthma, chronic bronchitis, chronic systemic inflammation or inflammatory respiratory disease.

In one embodiment, a diagnostic and/or prognostic method of assessing lung disease in a subject is provided, wherein the method includes use of two or more described gene biomarkers. In one aspect, the method includes detecting two or more gene biomarkers in a biological sample obtained from a subject expression. In another embodiment, the method includes measurement of the level of expression of a gene biomarker selected from: IL6R, CCR2, PPP2CB, RASSF2, WTAP, DNTTIP2, GDAP1, LIPE, and RPL14.

In another aspect, the present disclosure provides a method of monitoring an increase in the severity of lung disease in a subject by comparing expression profiles of two or more gene biomarkers in the subject at a first time point versus a second time point, wherein a difference in the expression profiles indicates an increase in severity of the subject's lung disease. In one embodiment, the gene biomarker is selected from: IL6R, CCR2, PPP2CB, RASSF2, WTAP, DNTTIP2, GDAP1, LIPE, and RPL14 (including sequences complementary to those encoding mRNAs).

In an additional aspect, the gene biomarkers are useful as prognostic indicators of lung disease. Thus, in one embodiment, the present disclosure provides a method of determining the prognosis of a lung disease in a subject by detecting in a subject sample expression of two or more gene biomarkers at a first point in time and then at a second point in time, and comparing the profile of gene biomarkers expressed at the second time point versus the first time point to determine the prognosis of the lung disease in a subject. In one embodiment, the gene biomarker is selected from: IL6R, CCR2, PPP2CB, RASSF2, WTAP, DNTTIP2, GDAP1, LIPE, and RPL14 (and complementary sequences thereof).

Also provided are kits for use in the diagnosis, prognosis and treatment of lung disease comprising one or more of the gene biomarkers or compositions described herein.

### BRIEF DESCRIPTION OF THE DRAWINGS

FIG. 1 shows candidate predictors sorted in decreasing order by mean decrease in accuracy (left panel) and mean decrease in Gini impurity (right panel).

FIG. 2 shows a set of top Database for Annotation, Visualization and Integrated Discovery (DAVID) annotated biological processes, fifteen in total, including the gene ontology category name, percentage of genes within the category, EASE score, and fold enrichment. Each category has an EASE score (p-value)<0.01 and a fold enrichment>1.5. ‘COPD LIST’ refers to genes identified by random forest; ‘Microarray’ refers to all the genes represented on the array.

FIG. 3 shows the DAVID annotated biological pathways, including the percentage of genes identified, EASE score and fold enrichment. Pathways have an EASE score (p-value)<0.01 and a fold enrichment>1.5. ‘COPD LIST’ refers to genes identified by random forest; ‘Microarray’ refers to all the genes represented on the array.

FIG. 4 shows some regulatory interactions between proteins and biological outcomes developed with Pathway Studio software. Panel 4A shows protein-protein interactions associated with the MAPK signaling cascade. Panel 4B shows protein-protein interactions associated with the apoptotic cascade. MAP2K4 can phosphorylate and activate MAPK1. Binding of MAP3K1 to TRAF2 can result in their subsequent activation providing two potential links between the two pathways depicted in Panels 4A and 4B (Chadee et al. 2002, Molecular and Cellular Biology 22:737-749; Witowsky & Johnson 2003, The Journal of Biological Chemistry 278:1403-1406). Random Forest (RF) model-identified genes are shown with the name surrounded by a dashed oval, the other genes are Pathway Studio-identified genes. The abbreviations for human genes and proteins appearing in this figure are from Pathway Studio.

FIG. 5 shows an example of gene expression results from an L₁ penalized logistic regression model. (A) Microarray results for the randomly selected samples from the training set (12 Controls and 12 Cases). Relative mRNA percent difference in expression is calculated using the Control group as the comparator, and p-value for difference between the Case/Control groups mean values obtained by Student's t-test. Asterisks indicate a p-value<0.05 (*), <0.01 (**) or <0.001 (***). (B) Real-time PCR is conducted on the same samples as in A. Relative mRNA expression levels are calculated using a ΔΔCt method algorithm. Asterisks indicate a p-value<0.05 (*) or <0.01 (**).

FIG. 6 shows a study flow diagram and clear descriptions of the cohort and training and test sets in the described COPD Biomarker Discovery Study.

### DETAILED DESCRIPTION

The present disclosure provides compositions and methods of identifying genes as biomarkers of lung disease and compositions and kits comprising materials (e.g., nucleic acids and/or protein affinity reagents such as antibodies) for use in assessing nucleic acid and protein expression from those genes. Also provided are methods of using the novel biomarker for diagnostic, prognostic and predictive measures of a subject's lung disease. In one embodiment, the lung disease is COPD, where by identifying genes differentially expressed in subjects with COPD compared to control subjects, (biomarkers for the diagnostic, prognostic and predictive measures of a subject's lung disease are provided). Other exemplary diseases include, but are not limited to, obstructive pulmonary disease, chronic systemic inflammation, emphysema, asthma, pulmonary fibrosis, cystic fibrosis, obstructive lung disease, pulmonary inflammatory disorder, and lung cancer.

In one embodiment an individual or a population of individuals may be considered as not having lung disease or impaired lung function when they do not have exhibit clinically relevant signs, symptoms, and/or measures of lung disease. Thus, in various aspects, an individual or a population of individuals may be considered as not having chronic obstructive pulmonary disease, chronic systemic inflammation, emphysema, asthma, pulmonary fibrosis, cystic fibrosis, obstructive lung disease, pulmonary inflammatory disorder, or lung cancer when they do not manifest clinically relevant signs, symptoms and/or measures of those disorders. In another embodiment, an individual or a population of individuals may be considered as not having lung disease or impaired lung function, such as COPD, when they have a FEV₁/FVC ratio greater than or equal to about 0.70 or 0.72 or 0.75. In another embodiment, an individual or population of individuals that may be considered as not having lung disease or impaired lung function are sex- and age-matched with test subjects (e.g., age matched to 5 or 10 year bands) that =current or former cigarette smokers without apparent lung disease who have an FEV1/FVC≧0.70 or ≧0.75. Individuals or populations of individuals without lung disease or impaired lung function may be employed to establish the normal range of proteins, peptides or gene expression. Individuals or populations of individuals without lung disease or impaired lung function may also provide samples against which to compare one or more samples taken from a subject (e.g., samples taken at one or more different first and second times) whose lung disease or lung function status may be unknown. In other embodiments, an individual or a population of individuals may be considered as having lung disease or impaired lung function when they do not meet the criteria of one or more of the above mentioned embodiments.

In one embodiment, control subjects, as that term is used herein are sex- and age-matched current or former cigarette smokers, without apparent lung disease who have FEV1/FVC≧0.70. Age matching may be conducted in bands of several years, including 5, 10 or 15 year bands. Control subjects are preferably recruited from the same clinical settings. A control group is more than one, and preferably a statistically significant number of control subjects. In one embodiment control subjects are sex- and age-matched (in 10 year bands) current or former cigarette smokers, without apparent lung disease who had FEV1/FVC≧0.70

In one embodiment, a control sample is a sample from one or more control subjects or which provides a result representative of tests conducted on a control group. In another embodiment, a control sample is a sample from a subject without lung disease (e.g., COPD) or which provides a result representative of tests conducted on a subjects without lung disease. In another embodiment a control sample is a sample containing a known amount (e.g., in mass, number of moles, or concentration) of one or more nucleic acids and/or proteins.

As described herein, a “gene biomarker” is a gene, or a nucleic acid sequence, such as the sequence of a gene, or fragment thereof, which is differentially expressed in a sample obtained from an individual having one phenotypic status (e.g., having a lung disease such as COPD) as compared with individual having another phenotypic status (e.g., control subject without a lung disease). A biomarker is an assayable nucleic acid sequence (or fragment thereof) that is used to identify, predict, or monitor a condition related to lung disease, such as COPD, or a therapy for such a condition, in a subject or sample obtained from a subject. The presence, absence, or relative amount of a gene biomarker can be used to identify a condition or status of a condition in a subject or sample obtained from that subject. Proteins that are encoded by a nucleic acid gene biomarker may be assayed as surrogates for the nucleic acid, and may be understood to be a biomarker or gene biomarker in that circumstance.

A gene biomarker may be characterized using a variety of approaches. Exemplary methodologies include, but are not limited to, the use of the polymerase chain reaction, sequencing, quantitative polymerase chain reaction, quantitative real-time polymerase chain reaction, protein or DNA array, microarray, ligase chain reaction, and oligonucleotide ligation assay, as well as use of high-throughput techniques such as cDNA microarray followed by statistical analysis to identify those nucleic acid sequences which are differentially expressed in subjects having lung disease as compared to control subjects.

A biomarker is differentially expressed between different phenotypic statuses if the expression level of the biomarker in the different groups is calculated to be statistically significantly different. Exemplary statistical analysis includes, among others, Random forest analysis (Breiman, 2001, Random Forests. Machine Learning 45:5-32), L₁ penalized logistic regression (Tibshirani, 1996, Journal of the Royal Statistical Society B 58:267-288) and use of R programming environment (R Development Core Team 2007, R: a language and environment for statistical computing. http://www R-project org).

Gene biomarkers, alone or in combination, are useful as diagnostic markers of: lung disease; determining therapeutic effectiveness of a treatment for lung disease and/or lung disease progression; determining prognosis of lung disease; and/or for determining an individual's relative risk of developing lung disease.

Methods for identifying gene biomarkers are useful as diagnostic or prognostic indicators of different classifications and/or severity of lung disease by comparison of gene biomarkers differentially expressed in subjects having lung disease varying in degrees of severity or symptoms. In one embodiment, the gene biomarkers of lung function may be used as prognostic indicators of how likely a subject having lung disease is to experience an increase in disease symptoms or how severe those symptoms may become. In one embodiment, the greater the difference in expression of the gene biomarkers of lung function (e.g., IL6R, CCR2, PPP2CB, RASSF2, WTAP, DNTTIP2, GDAP1, LIFE, and RPL14) in a subject with suspected lung disease from when compared to control subjects, the more likely they will have the disease.

Gene biomarkers may also be identified by analysis of nucleic acid sequences differentially expressed by a subject with a lung disease as compared to nucleic acid sequences expressed by gender-matched control subjects. Identification of nucleic acid sequences that are differentially abundant among subjects with lung disease as compared to control subjects (e.g., COPD subjects having mild to moderate COPD with rapid or slow decline in lung function versus age- and gender-matched smokers without COPD) allows an understanding of the mechanisms underlying a lung disease and its related decline in lung function. Such nucleic acid sequences are useful as gene biomarkers for diagnostic and prognostic determinants of lung disease and/or assessing a subject's relative risk of developing a lung disease.

In one embodiment, methods for determining gene expression profiles include determining the amount of RNA that is produced by a gene encoding a polypeptide. Such methods include, but are not limited to, the use of reverse-transcriptase PCR (RT-PCR), competitive RT-PCR, real time RT-PCR, differential display RT-PCR, Northern Blot analysis and other related assays. The methods include the use of individual PCR reactions as well as amplification of complementary DNA (cDNA) and/or complementary RNA (cRNA) produced from mRNA and analysis via microarray.

Gene expression profiling using microarray analysis allows measurement of the steady-state mRNA level of thousands of genes simultaneously. Microarray techniques useful in the methods described herein are known in the art and are described, for example, in U.S. Pat. No. 6,271,002; U.S. Pat. No. 6,218,122; U.S. Pat. No. 6,218,114; and U.S. Pat. No. 6,004,755.

A gene biomarker may be detected in any tissue of interest from a subject suspected of having, at risk of having, or diagnosed as having a lung disease. Biological samples obtained from a subject that are suitable for detection of gene biomarkers include, but are not limited to, serum, plasma, blood, lymphatic fluid, cerebral spinal fluid, saliva, and epithelial cells, such as those available from a buccal swab. It is known that the transcriptome of peripheral blood leukocytes (PBL) reflect a majority of genes actively expressed in a subject. Thus, PBLs are useful as a target tissue “surrogate” for identifying genes differentially expressed in diseased subjects as compared to control subjects. As such, the present disclosure also provides a method of identifying the presence of a gene biomarker in a biological sample of a subject obtained using less invasive sampling techniques. A biological sample includes peripheral blood cells which are readily accessible using traditional blood drawing techniques such as, for example, venipuncture or finger prick.

In one embodiment, a gene biomarker of lung disease is selected from the nucleic acid sequence of a gene set forth in Supplementary Table II. In another embodiment, a gene biomarker of lung disease is a nucleic acid sequence encoding IL6R, CCR2, PPP2CB, RASSF2, WTAP, DNTTIP2, GDAP1, LIPE and RPL14, or a complementary sequence thereof (i.e., IL6R complementary sequence, CCR2 complementary sequence, PPP2CB complementary sequence, RASSF2 complementary sequence, WTAP complementary sequence, DNTTIP2 complementary sequence, GDAP1 complementary sequence, LIPE complementary sequence and RPL14 complementary sequence), or a fragment thereof.

In another embodiment, the present disclosure provides a composition comprising two, three, four, five, six, seven, eight or nine nucleic acid molecules, wherein each nucleic acid molecule differs from the other nucleic acid molecules and each nucleic acid molecule comprises a nucleotide sequence that is selected independently from the nucleic acid sequences of the genes set forth in Supplementary Table II, their complements, or a sequence having 70-99% identity to the nucleic acid sequences of the genes set forth in Supplementary Table II, or a fragment thereof. Moreover, such a composition may contain two, three, four, five, six, seven eight or nine nucleic acid molecules that are directed to different sequences selected independently from the nucleic acid sequences of the genes set forth in Supplementary Table H, or a sequence having 70-99% identity to the nucleic acid sequences of the genes set forth in Supplementary Table II, or a fragment thereof. It is understood that such nucleic acid molecules may have the sequence of the coding strand or the non-coding strand of the gene, or a fragment thereof. In aspects of such an embodiment, the fragments may be selected independently to have lengths greater than about 20, 22, 23, 24, 25, 26, 27, 28, 32, 34, 36, 38, 40, 50, 60, 75, 100, or 150 contiguous nucleotides of those sequences.

In another embodiment, the present disclosure provides a composition comprising two, three, four, five, six, seven, eight or nine different nucleic acid molecules where each comprises a nucleotide sequence that is: complementary to a fragment greater than about 20, 22, 23, 24, 25, 26, 27, 28, 32, 34, 36, 38, 40, 50, 60, 75, 100, or 150 contiguous nucleotides of the coding or non-coding strand of a gene set forth in Supplementary Table II, an RNA or cDNA transcribed from a gene set forth in Supplementary Table II, or the protein coding (exons) thereof.

Nucleic acid molecules, which may also be referred to herein as polynucleotides, “polynucleotide probes” or simply as “probes” may be immobilized on a substrate. In one embodiment, the present disclosure provides a device comprising one or more nucleic acid molecules immobilized on a substrate wherein each probe includes a gene biomarker. In another embodiment, the device comprises a plurality of nucleic acid molecules, each probe stably associated with (e.g., covalently bound to) and having a unique position on the substrate. In one embodiment, the substrate comprises an array or microarray device. In yet another embodiment the array comprises an array of nucleic acid molecules wherein two, three, four, five, six, seven, eight or nine different nucleic acid molecules are gene biomarkers of lung disease described herein (e.g., IL6R, CCR2, PPP2CB, RASSF2, WTAP, DNTTIP2, GDAP1, LIFE, and RPL14).

Nucleic acid molecules comprising a nucleotide sequence of a gene biomarker of lung disease may also be immobilized on beads or nanoparticles, such as gold, platinum, or silver nanoparticles. Nucleic acid molecules comprising a nucleotide sequence of a gene biomarker of lung disease may also be detectably labeled. In one embodiment, the label is detectable by fluorescence, or UV/Visible spectroscopic means. In other embodiments, the label is a nanoparticle such as a colloidal metal nanoparticle that is detectable by spectroscopic means including plasmon resonance. In still other embodiments, the label is a radioactive label.

Another embodiment is directed to a device comprising two, three, four, five, six, seven or eight different nucleic acid molecules that comprise the sequence of a gene biomarker of lung disease. In one embodiment the nucleic acid molecule(s) comprises a nucleotide sequence having greater than about 20, 22, 23, 24, 25, 26, 27, 28, 32, 34, 36, 38, 40, 50, 60, 75, 100, or 150 contiguous nucleotides of a gene biomarker of lung disease set forth in Supplementary Table II. In such embodiments the device can be an array wherein each nucleic acid molecule is fixed at a spatially addressable location.

The disclosure provided herein employs highly sensitive techniques for identification of gene biomarkers. that have low systemic levels in a subject. In one embodiment, a biological sample may be analyzed by use of an array technology and methods employing arrays such as, for example, a nucleic acid microarray or a biochip bearing an array of nucleic acids. An array or biochip generally comprises a solid substrate having a generally planar surface, to which a capture reagent is attached. Frequently, the surface of an array or biochip comprises a plurality of addressable locations, each of which has a capture reagent bound thereon. In one embodiment the arrays will permit the detection and/or quantitation of two, three, four, five, six, seven, or eight or more different biomarkers associated with COPD or its progression. In another embodiment the array will comprise addressable locations for capturing/binding and/or measuring two, three, four, five, six, seven, eight or more different gene biomarkers of lung disease. In one embodiment the gene biomarkers of lung disease are selected from nucleic acid sequences of one or more genes selected from IL6R, CCR2, PPP2CB, RASSF2, WTAP, DNTTIP2, GDAP1, LIPE and RPL14 (including the coding strand, non-coding strand, or exons thereof).

In one particular embodiment, the methods are provided using one or more gene biomarkers for diagnosing the presence of a lung disease or for determining a risk of developing a lung disease in a subject. A gene biomarker may include a nucleic acid sequence or fragment thereof encoding IL6R, CCR2, PPP2CB, RASSF2, WTAP, DNTTIP2, GDAP1, LIPE, RPL14, IL6R complementary sequence, CCR2 complementary sequence, PPP2CB complementary sequence, RASSF2 complementary sequence, WTAP complementary sequence, DNTTIP2 complementary sequence, GDAP1 complementary sequence, LIPE complementary sequence or RPL14 complementary sequence. A lung disease may include, but is not limited to, asthma, COPD, lung cancer, alpha-1 antitrypsin deficiency, respiratory distress syndrome, chronic bronchitis, chronic systemic inflammation, and inflammatory respiratory disease, which may, or may not, include lung cancer in any embodiment described herein. In one aspect the biological sample is a blood sample, a plasma sample, a serum sample, a urine sample, a lymphatic fluid sample, saliva sample or a sputum sample.

In one aspect, the present disclosure provides a method for identifying gene biomarkers of a disease that are associated with either a slow decrease or a rapid decrease in lung function. Methods are also provided for discriminating between a rapid and a slow decline in lung function and/or methods for identifying a subject as having an increased risk of developing a rapid decline in lung function or an increased risk of developing a slow decline in lung function by use of a gene biomarker. As used herein, the term “increased risk” refers to a statistically higher frequency of occurrence of the disease or disorder in an individual in comparison to the ?average frequency of occurrence of the disease or disorder in a population. A “decreased risk” refers to a statistically lower frequency of occurrence of the disease or disorder in an individual in comparison to the ?average frequency of occurrence of the disease or disorder in a population.

In another embodiment, the status of a subject's lung disease may be determined by measuring the quantity of one or more particular gene biomarkers present in a biological sample from that subject, and correlating the quantity of each biomarker with a previously determined measure of the severity of the disease based on the presence and/or quantity of one or more particular gene biomarkers present in a test sample from the subject. As used herein, the term “status” refers to the degree of severity of a subject's lung disease such as, for example, the number or degree of severity of symptoms presented or exhibited by the subject with the lung disease. The symptoms associated with different forms of lung diseases may differ between forms of lung diseases or may overlap. For example, exemplary symptoms commonly associated with COPD include, destruction or decreased function of the air sacs in the lungs, cough producing mucus that may be streaked with blood, fatigue, frequent respiratory infections, headaches, dyspnea, swelling of extremities, and wheezing. A subject with COPD may have a few to all of these symptoms. A subject with an early stage of COPD may exhibit one, two, three, or only a few of those symptoms.

In another embodiment, the present disclosure provides a method of determining the status of a subject's lung disease by assessing the level of expression of one or more gene biomarkers during the course of the subject's lung disease. Such assessment includes (1) measuring at a first time point the level of expression of one or more gene biomarkers of lung disease in a subject's sample, (2) measuring the same biomarker(s) at a second time, and (3) comparing the first measurement to the second measurement, wherein a difference between the two measurements indicates the status of the lung disease, such as an increase or decrease in severity of the disease. In one embodiment a gene biomarker of a lung disease or an impaired lung function measure is selected from the group consisting of: IL6R, CCR2, PPP2CB, RASSF2, WTAP, DNTTIP2, GDAP1, LIPE, RPL14, or fragments thereof. In other aspects the method further comprises measuring two, three, four, five, six, seven, or eight, or more different gene biomarkers of lung disease.

Techniques for use in a method of measuring an increased or decreased expression of gene biomarkers include the use of quantitative assays for nucleic acids and proteins, including for example, polymerase chain reaction, array detection and measurement of proteins (e.g., using immobilized antibodies), quantitative RT-PCR (reverse transcriptase followed by PCR for measuring mRNA for example), quantative real time PCR, multiplex PCR, quantitative DNA array analysis, autoradiograph analysis, quantitative hybridization, immunoassays (e.g., ELIAS, Western, or sandwich assays), quantitative rRNA-based amplification, fluorescent probe hybridization, fluorescent nucleic acid sequence specific amplification, loop-mediated isothermal amplification and/or ligase chain reaction.

In one embodiment, the present disclosure provides a method of managing a subject's lung disease whereby a therapeutic treatment plan is customized/personalized or adjusted based on the status of the disease. Exemplary therapeutic treatments for lung disease include administering to the subject one or more of: immunosuppressants, corticosteroids (e.g., betamethasone delivered by inhaler), β2-adrenergic receptor agonists (e.g., short acting agonists such as albuterol), anticholinergics (e.g., ipratropium, or a salt thereof delivered by nebulizer), and/or oxygen. In addition, where the lung disease is caused or exacerbated by bacterial or viral infections, one or more antibiotics or antiviral agents may also be administered to the subject.

The materials and reagents required for diagnosing a lung disease, for determining the prognosis of a lung disease, or for use in the treatment or management of lung disease in a subject may be assembled together in a kit. A kit comprises one or more biomarker probes and a control nucleic acid sequence (e.g., present in a known quantity or amount), wherein the control nucleic acid sequence corresponds to a sequence that is not a gene biomarker of lung disease. The kit may be used for diagnosing, identifying prognosis, and/or predicting a lung disease in a subject. The kit generally will comprise components and reagents necessary for determining one or more biomarkers in a biological sample as well as control and/or standard samples. For example, a kit may include, probes, and/or antibodies specific to the one or more proteins, or peptide fragments of proteins, encoded by a gene set forth in Supplementary Table II for use in a quantitative assay such as RT-PCR, in situ hybridization, microarray and/or biochip detection. In another embodiment, the kit may include a compositions with gene expression products in ratios found in individuals having lung disease and/or compositions with gene expression products in ratios found in individuals not having a lung disease, thus avoiding the use of control gene(s) or control sample(s) from “control” subjects. In some embodiments, the kit includes a pamphlet which includes a description of use of the kit in relation to COPD diagnosis, prognosis, or therapeutic management and instructions for analyzing results obtained using the kit.

### EXAMPLES

A cDNA microarray was used to obtain data to identify genes differentially expressed in PBLs between adult cigarette smokers or other subjects with or without COPD. In a training set of Cases and Controls clearly defined by spirometric criteria, random forest statistical modeling was used to generate a list of variables that predicted COPD classification. This list was then subjected to an L₁ penalized logistic regression model to create a more focused set of variables. Both lists were assessed in a test set of subjects with spirometric parameters that closely bordered the generally acceptable spirometric diagnostic value for COPD. The identified genes were analyzed for their ontology assignment and pathway involvement. The gene expression profiles identified in this study are novel biomarkers for COPD and provide insight into disease mechanisms.

### Materials and Methods

Study Design and Subjects

The COPD Biomarker Discovery Study (CBD) included male and female self-reported cigarette smokers, aged 45 years or older, with at least 10 pack-years smoking history that were recruited from the University of Utah Health Sciences Network of local clinics and hospitals and from community physician offices. COPD was diagnosed in 300 subjects according to the Global Initiative for Chronic Obstructive Lung Disease (GOLD) spirometric guidelines as having a ratio of forced expiratory volume in 1 second (FEV₁) to forced vital capacity (FVC)<0.70 (Rabe et al. 2007, American Journal of Respiratory and Critical Care Medicine 176:532-555). The Control group included 425 sex- and age-matched, current or former cigarette smokers, without apparent lung disease with FEV₁/FVC≧0.70. Individuals who had recent exacerbation of COPD, uncontrolled angina, hypertension, or allergy to albuterol, and females who were pregnant or lactating were excluded. Demographic variables, respiratory symptoms, medical history, tobacco use history, and concomitant medications were assessed. Pack-years were calculated as (maximum average cigarettes smoked per day over total smoking history/20)×(total years smoking). Body weight and height were measured. Spirometry was performed with a rolling seal spirometer by certified pulmonary function technicians according to American Thoracic Society guidelines (Miller et al. 2005, European Respiratory Journal 26:319-338). Measurements of FEV₁ and FVC were made before and at least 20 min after inhaled bronchodilator administration (albuterol 180 μg). The FEV₁/FVC ratio was calculated for each subject from the highest post-bronchodilator values of FEV₁ and FVC. A blood sample was collected for assessment of carboxyhemoglobin (COHb) and complete blood cell counts. In a subgroup of 81 subjects with COPD and 61 unaffected (Control) subjects, a whole blood sample was also obtained for assessment of gene expression in PBLs.

Blood Sample Collection and Processing

Whole blood samples were obtained from each subject by venipuncture using 10 mL EDTA Vacutainer® tubes (BD, Franklin Lakes, N.J., USA). COHb, hemoglobin, hematocrit and total and differential white blood cell (WBC) counts were measured at ARUP Laboratories™, a national, CLIA (Clinical Laboratory Improvement Amendments of 1988)-certified reference laboratory (Centers for Medicare & Medicaid Services 1992, Federal Register 40:7002-7186). Isolation of PBLs was carried out using the LeukoLOCK™ Total RNA Isolation System (Ambion, Inc., Austin Tex., USA) following the manufacturer's protocol. Briefly, after isolation of PBLs, the filter was flushed with 3 EA. of phosphate-buffered saline, to remove residual red blood cells, and then with RNAlater®, to stabilize the leukocyte RNA, and frozen at −20° C. until processing for RNA. RNA isolation was then carried out using the mirVana™ miRNA Isolation Kit (Ambion, Inc., Austin Tex., USA). The LeukoLOCK™ filter was flushed with 2.5 mL of mirVana miRNA Lysis Solution, and the lysate was collected in a 15-mL conical tube. mirVana miRNA homogenate additive (one-tenth volume) was then added to the cell lysate. A volume of acid-phenol:chloroform, equal to the lysate volume, was used to flush the LeukoLOCK™ filter and was collected into the same 15-mL conical tube as the lysate. The tube was shaken vigorously for 30 seconds and stored for 5 min at room temperature. The samples were centrifuged for 10 min at 10,000×g (maximum) in a table-top centrifuge. The aqueous phase was transferred into a new tube, and mixed with 1.25 volumes of room-temperature 100% ethanol, and the mixture was filtered through the filter cartridge into the collection tubes supplied with the kit. The isolated RNA was then washed and eluted following the standard steps described in the kit's manual. Quality of the isolated RNA was checked using the Agilent 2100 Bioanalyzer (Agilent Technologies, Inc., Santa Clara, Calif., USA) before use and storage at −80° C.

Microarray Data Acquisition

Statistical procedures and analysis involved in pre-processing and identifying differential expression of microarray data were performed using Bead Studio® v3.0.14 (IIlumina Inc., San Diego, Calif., USA) and R-2.6.1 software (R Development Core Team 2007). cRNA from each sample following RNA isolation were hybridized to Sentrix® Human WG-6 BeadChips (Illumina Inc., San Diego, Calif., USA). Hybridized BeadArrays™ were examined with respect to number of genes detected, average intensity, 95th percentile of signal intensity, signal-to-noise ratio, and background signal intensity as a means of assessing quality. For each quality control (QC) measure, the BeadArray statistics were plotted and the mean+3 standard deviations were overlaid on the plot as a method for identifying potentially outlying arrays. All BeadArrays were considered to be within acceptable limits for these QC measures. In addition, the BeadArrays were examined with respect to beadtypes labeled as hybridization, low and high stringency, biotin, housekeeping, and labeling controls (data not shown). All control beadtypes yielded intensities at the expected levels, therefore each of the 142 hybridizations were considered to be of good quality.

Microarray Data Preprocessing

Prior to analysis, the gene expression data was log₂ transformed. Since negative control bead background correction was demonstrated to negatively impact identifying differentially expressed genes (Dunning et al. 2008, BMC Bioinformatics 9:85), the estimated background from the negative control beads was not subtracted from the mean beadtype signal intensities. The log₂ transformed intensities were subsequently normalized using a global median scaling method. Specifically, the expression for each sample was scaled by an array-specific constant factor so that the median expression values were the same across all arrays. An arbitrarily selected array was set as the baseline against which all other arrays were normalized. For array i and beadtype j, using the log₂ transformed expression values log₂ (xᵢⱼ), global normalization was performed as follows: 1) the median expression for the baseline array

$${\tilde{x}}_{base} = {median}_{j} ( {log}_{2} ( {x}_{base , j} ) ) ,$$

was calculated; 2) for the i^{th} array, the median expression,

$${\tilde{x}}_{i} = {median}_{j} ( {log}_{2} ( {x}_{ij} ) ) ,$$

was also calculated; and 3) for the i^{th} array, bᵢ=\tilde{x}_{base}/\tilde{x}ᵢ was taken to be the global scaling factor and was applied to normalize the j expression values for array i so that the log₂ transformed and scaled values for beadtype j and array i were xᵢⱼ^{norm}=bᵢ log₂ (xᵢⱼ).

Random Forest Analysis

The normalized gene expression data were combined with selected demographic, smoking history and clinical variables (see Supplementary Table I). A random forest consisting of 10,000 trees was derived for predicting COPD-affected (Case) or unaffected (Control) samples/individuals, using a split-sample approach (training and test sets) and the random Forest package in the R programming environment (Breiman 2001, Liaw & Wiener 2002, R News 2:18-22; R Development Core Team, 2007). An extreme discordant phenotype design (Zhang et al. 2006, Pharmacogenitics and Genomics 16:401-413), based on the FEV₁/FVC ratio, was used to select the training set for the analysis. Of 142 subjects, 36 were clearly classified as having COPD (FEV₁/FVC<0.60), and 36 were classified as Controls (FEV₁/FVC>0.75). This set of samples was then used as the training set for the analysis in order to maximally stratify the Case and Control subgroups. The remaining 70 subjects had FEV₁/FVC values between 0.60 and 0.75 and were used as the test set.

For each classification tree in the random forest, the observations left out of the bootstrap re-sample (e.g., “out-of-bag”) were used as a natural test set for estimating prediction error. The out-of-bag observations were also used to estimate the importance of each variable for the classification task (Archer & Kimes, 2008, Computational Statistics and Data Analysis 52:2249-2260). The bootstrap method was used to estimate the null distribution for the mean decrease in Gini impurity by drawing a random sample with replacement from those variables with a non-zero mean decrease in Gini impurity, estimating the mean decrease of the re-sampled observations and repeating this procedure 2000 times. Candidate predictors with a Gini impurity>99.99795% were considered significant for the classification task.

L₁ Penalized Logistic Regression

An L₁ penalized logistic regression model was fit to predict the dichotomous outcome variable (Case/Control status) using the significant candidate predictors identified by the random forest algorithm. This additional modeling step was used to identify a more focused set of predictor variables that retain a similar error rate as the complete predicted random forest. This model was fit using the same training set used to derive the random forest model. The glmpath library (Park & Hastie, 2007, Journal of the Royal Statistical Society B 69:659-677) in the R programming environment (R Development Core Team, 2007) was used for fitting the L₁ penalized models. The final model was selected as that model with minimum Akaike's information criterion (AIC) and was subsequently used to obtain fitted probabilities for all testable subjects. Those subjects with probabilities≧0.5 were classified as Cases, and all others were classified as Controls.

Gene Ontology and Pathway Analysis

Genes identified statistically as having significant predictive value for the discrete Case/Control outcome were used as the input for subsequent gene ontology and pathway analysis. Gene ontology and functional categories were identified by analyzing isolated gene lists using the Database for Annotation, Visualization and Integrated Discovery (DAVID, on the world wide web at david.abcc.ncifcrf.gov/) (Dennis et al. 2003, Genome Biology 4:3) and Pathway Studio V5.0 (Ariadne Inc., Rockville, Md., USA). EASE scores for gene-enrichment analysis were calculated using a 0.1 threshold. The DAVID annotation tool was also used to probe the Kyoto Encyclopedia of Genes and Genomes (KEGG, www.genome.jp/kegg/kegg2.html), BioCarta (www.biocarta.com/genes/index.asp) and the Biological and Biochemical Image Database (BBID, on the world wide web at bbid.grc.nia.nih.gov/) pathway databases to identify regulated pathways and to complement the gene ontology. “Biological processes” and “Pathways” with ap-value≦0.05 were considered significant. The output analyses were manually filtered to remove overlapping and redundant categories to generate non-redundant lists.

Quantitative Real-Time PCR (qRT-PCR)

Quantitative real-time polymerase chain reaction (qRT-PCR) was performed on isolated RNA from randomly selected subjects in the training set (12 with and 12 without COPD) to confirm the microarray results in terms of differential expression and statistical significance. First-strand cDNA was synthesized from 1 μg of RNA in a 100 μl reaction volume with the TaqMan® Reverse Transcriptase Reaction Kit (Applied Biosystems, Carlsbad, Calif., USA) using random hexamers as primers following the manufacturer's recommended protocol. After the synthesis was complete, the cDNA was diluted 1:3. Six microliters of diluted cDNA were then used for each qRT-PCR reaction in a final volume of 20 using pre-designed Gene Expression Assays (Applied Biosystems, Carlsbad, Calif., USA) for the genes of interest. All PCR reactions were carried out in triplicate. Relative expression levels were calculated using the ΔΔCt method algorithm provided by Applied Biosystems. The average intensity value obtained for the Control subjects was used as the calibrator. All reactions were run in an Applied Biosystems 7500 Fast Sequence Detection System (Applied Biosystems, Carlsbad, Calif., USA). The gene expression assays used were: 18S (Hs99999991_{—}1), GAPDH (4310884E), DNTTIP2 (Hs00966646_{—}1), GDAP1 (Hs00184079_{—}1), IL6R (Hs01075667_{—}1), LIPE (Hs00943410_{—}1), WTAP (Hs00374488_{—}1), CCR2 (Hs00174150_{—}1), PPP2CB (Hs00602137_{—}1), RASSF2 (Hs00542460_{—}1) and RPL14 (Hs00427856_{—}1).

### Results

Subject Demographics

Characteristics of the spirometrically defined COPD-affected and unaffected groups (overall and for the training set) are summarized in Table I. The distribution of the COPD group by severity of airflow obstruction (FEV₁ as percent of predicted) by GOLD spirometric guidelines (Rabe et al. 2007) was GOLD 1 (mild, n=30), GOLD 2 (moderate, n=38), GOLD 3 (severe, n=6), and GOLD 4 (very severe, n=7). It should be noted that 10 subjects with FEV₁/FVC>0.70 were categorized as Controls according to the GOLD guideline but had subnormal FEV₁ (<80% predicted) and could be considered to have spirometrically indeterminate Case/Control status; 3 subjects were in the training set, and 7 were in the test set. In the cohort overall and in the training and test sets, the COPD group was older and had at least 56% greater pack-years of cigarette smoking, on average, than the Control group. However, the proportion of current smokers was similar across all groups, at 58-69%. Although the mean total circulating WBC count did not differ significantly between the groups, those with COPD had significantly higher mean neutrophils and lower mean lymphocytes, as percentages of the total WBC, than the group without COPD.

TABLE I
Characteristics of the spirometrically defined COPD-affected (Cases)
and unaffected (Controls) subjects.

| | All Subjects | | | Training Subset^{a} | | |
|---|---|---|---|---|---|---|
| | Cases | Controls | | Cases | Controls | |
| Characteristic | (n = 81) | (n = 61) | p-value^{b} | (n = 36) | (n = 36) | p-value^{b} |
| Male (%) | 67 | 62 | 0.60 | 64 | 61 | 1.00 |
| Age (y) | 61.2 (8.2) | 54.8 (9.0) | <0.0001 | 63.3 (7.4) | 52.6 (7.7) | <0.0001 |
| Current smoker (%) | 62 | 64 | 0.86 | 58 | 69 | 0.46 |
| Cigarettes per day^{c} | 14.6 (17.0) | 12.0 (12.3) | 0.30 | 12.7 (14.1) | 13.0 (13.4) | 0.92 |
| Pack-years | 59.5 (38.0) | 38.1 (19.8) | <0.0001 | 64.3 (38.8) | 32.8 (19.3) | <0.0001 |
| FEV₁ (L) | 2.33 (1.01) | 3.12 (0.79) | <0.0001 | 1.74 (0.94) | 3.30 (0.75) | <0.0001 |
| FEV₁ (% predicted) | 70.6 (24.9) | 94.6 (14.3) | <0.0001 | 54.2 (23.5) | 99.0 (14.1) | <0.0001 |
| FVC (L) | 4.05 (1.32) | 4.04 (1.01) | 0.94 | 3.8 (1.47) | 4.1 (0.97) | 0.32 |
| FEV₁/FVC (%) | 56.3 (12.9) | 77.4 (4.9) | <0.0001 | 44.7 (11.1) | 80.8 (3.1) | <0.0001 |
| WBC, total (10³ μL⁻¹) | 7.4 (1.7) | 7.6 (2.1) | 0.57 | 7.6 (1.9) | 7.3 (1.8) | 0.51 |
| Granulocytes (%) | 64 (7) | 59 (10) | 0.004 | 66 (6) | 57 (10) | <0.0001 |
| Lymphocytes (%) | 25 (7) | 30 (9) | 0.002 | 23 (6) | 32 (10) | <0.0001 |
| Monocytes (%) | 6.2 (1.7) | 5.9 (1.6) | 0.19 | 6.4 (1.7) | 5.7 (1.4) | 0.06 |

COPD, chronic obstructive pulmonary disease;
FEV₁, forced expiratory volume in 1 s;
FVC, forced vital capacity;
WBC, white blood cells.
Values are mean (±SD) unless otherwise indicated.
^{a}COPD subjects with % FEV₁/FVC <60 and control subjects with % FEV₁/FVC >75.
^{b}p-value for difference in mean values between the Case/Control groups was obtained by Welch's t-test for continuous variables and by Fisher's exact test for categorical variables.
^{c}Average daily cigarette consumption of current smokers during the 3 months prior to study participation

Identification of COPD Predictors

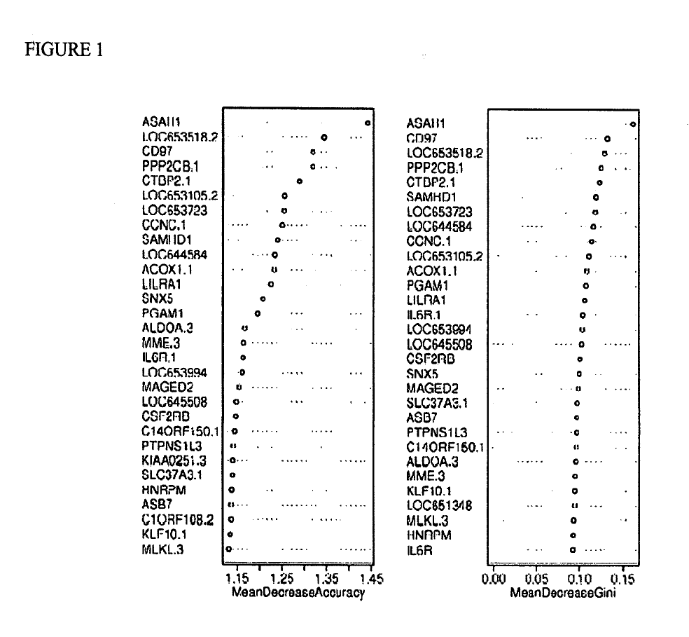

Due to the inability of the random forest algorithm to handle missing values among the predictor variables, the medication history of the subjects was not included in the analysis since several subjects had missing values. For example, 15/81 (18.5%) Cases and 19/61 (31%) Controls failed to indicate whether they were using glucocorticoids. The final size of the training set was 33 Cases and 34 Controls because 3 Cases and 2 Controls had missing values for other key variables. The out-of-bag estimate of error associated with the random forest analysis in the training set was 6.0% overall, with a misclassification rate of 2.9% for the spirometric Controls and 9.1% for the spirometric Cases (Table H). The random forest algorithm identified 1,014 candidate predictor variables, which included only 1 phenotypic variable, ‘years of daily smoking’. The top 30 candidate predictors using the mean decrease in Gini impurity, as well as the mean decrease in accuracy, are displayed in FIG. 1. The complete list of predictors can be found in Supplementary Table H.

TABLE II
Spirometric class versus random forest model-predicted class with associated class-specific discordance
rates for the training set (FEV₁/FVC <0.60 or >0.75) and the test set (FEV₁/FVC 0.60-0.75).

| | Spirometric class | | | |
|---|---|---|---|---|
| | Training set (n = 67) | | Test set (n = 65) | |
| | Cases | Controls | Cases | Controls |
| Predicted Class | | | | |
| Cases | 30 | 1 | 27 | 2 |
| Controls | 3 | 33 | 14 | 22 |
| Discordance rate (%) | 9.1 | 2.9 | 34.1 | 8.3 |

FEV₁, forced expiratory volume in 1 s;
FVC, forced vital capacity

The random forest model derived using the training set was then applied to the remaining 70 subjects with FEV₁/FVC values of 0.60-0.75 (test set). Five subjects were excluded due to missing values for a key variable, leaving 65 subjects as a test set for evaluation of the random forest classifier. The overall misclassification rate for the test set was 24.6% (16/65). Spirometric versus gene expression-predicted classifications for the training and test sets are shown in Table II, along with misclassification rates. Of the discordantly classified subjects in the test group, 14/16 (87.5%) were classified as Cases by spirometry but not by their gene expression profile.

Gene Ontology and Pathway Analyses

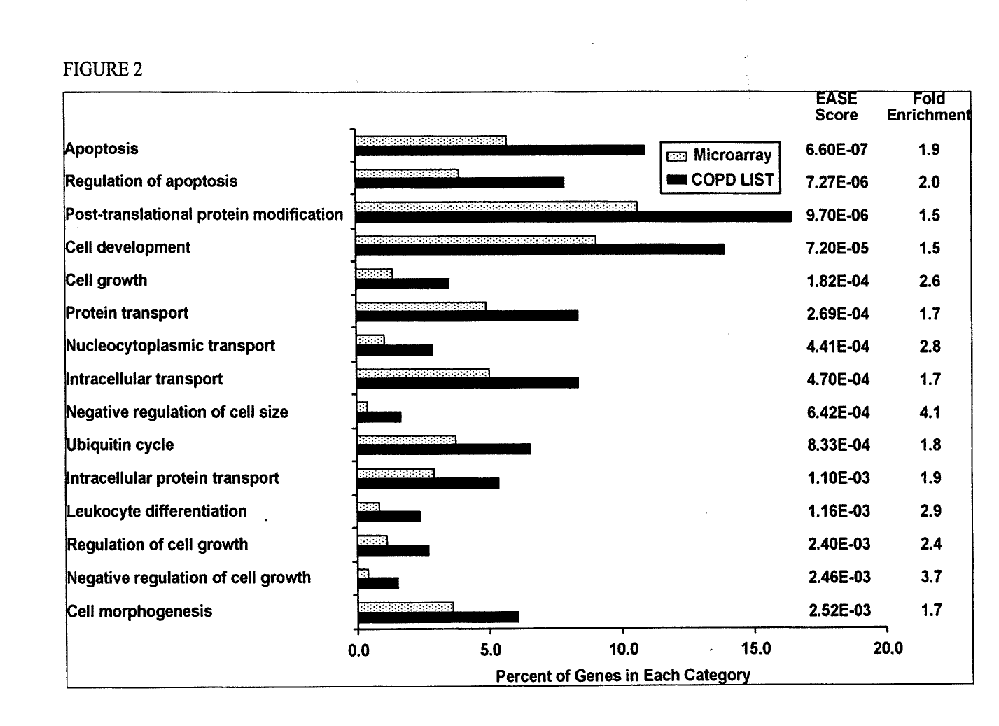

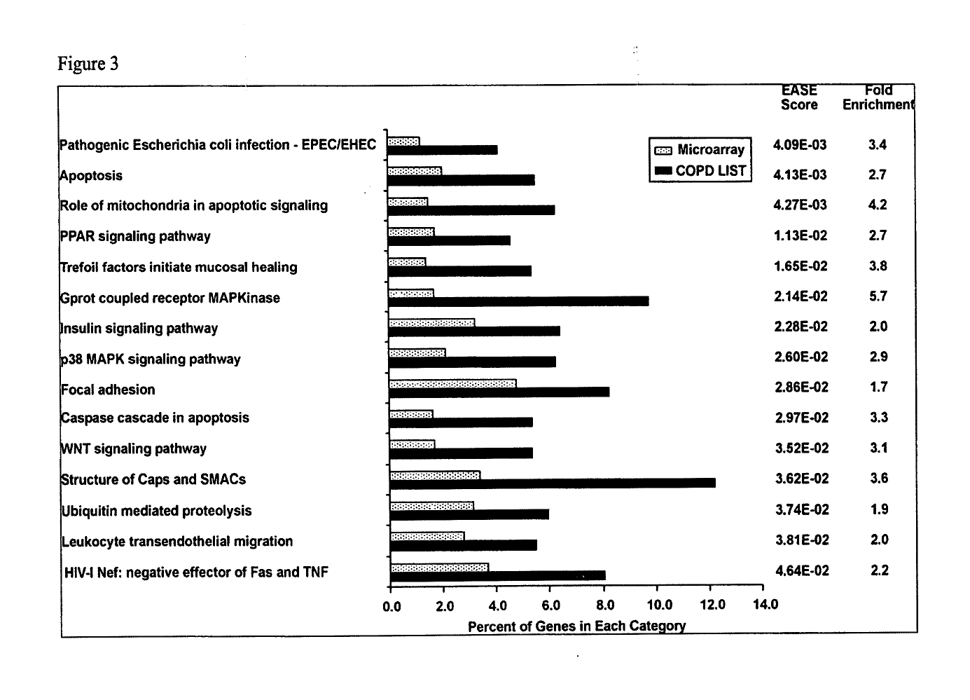

In an effort to identify biological processes and pathways that were differentially affected in Cases versus Controls, gene ontology assessment using the DAVID annotation tool (Dennis et al., 2003) was performed. A total of 784 genes (77.4% of the 1,013 genes identified by random forest modeling) were represented in the DAVID gene ontology categories. The analysis output list was manually edited to remove redundant and overlapping gene ontologies. Biological processes that were enriched in the set of predictor genes included regulation of apoptosis and cell growth, macromolecule (protein and RNA) transport, post-translational protein modification, cellular defense response, inflammatory response and RNA processing (FIG. 2). Major pathways identified by DAVID included apoptosis (mitochondrial apoptotic signaling and caspase cascade), p38 MAPK, WNT and PPAR signaling, focal adhesion and leukocyte transendothelial migration (FIG. 3).

The gene ontology analysis revealed a number of up-regulated genes involved in positive regulation of apoptosis (e.g., BAD, CASP4, CASP6, CASP10, DIABLO, FAF1, FASTK and TRADD) as well as a number of genes involved in inhibition of apoptosis (e.g., BCL2L1, BIRC2, CDKN2D, MCL1, NAIP, SERPINB2, SGMS1 and YWHAZ). A similar situation occurred with cell cycle progression related genes. Several of the genes identified are involved in general regulation of the cell (e.g., CCT7, CDC2L1, CDK2, CDC42, CDKN2D, MDM4, NEDD9, PCNA, PML, PMS1, RASSF2, RASSF4, RASSF5, RB1, TSC1, VEGFB and VHL) with a number of them clearly involved in negative regulation of the cell (e.g., CDKN2D, PML, RASSF2, RASSF4, RB1 and TSC1).

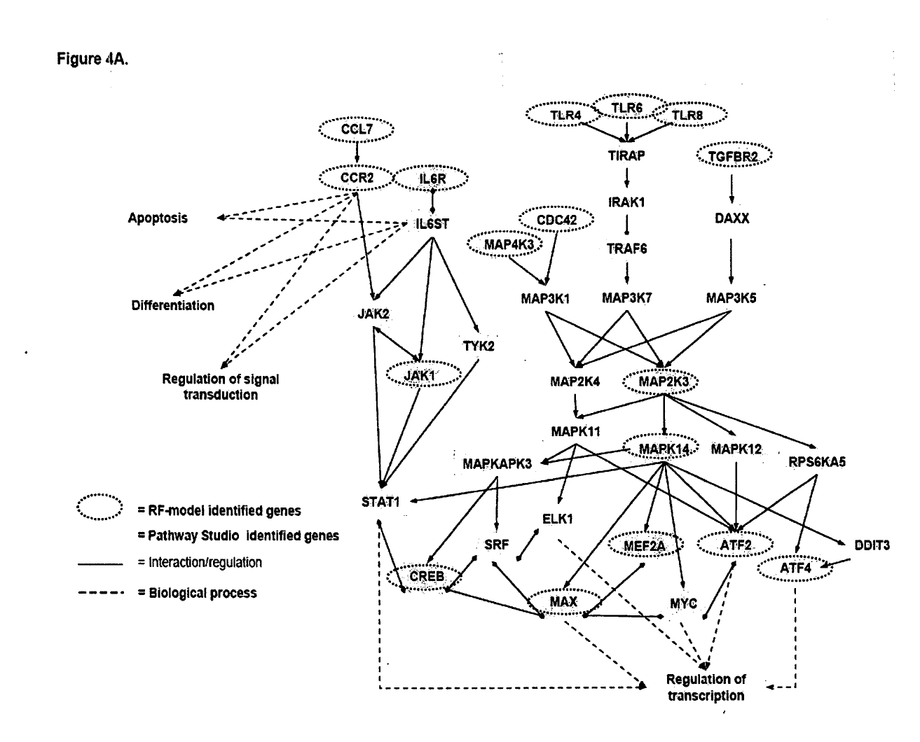

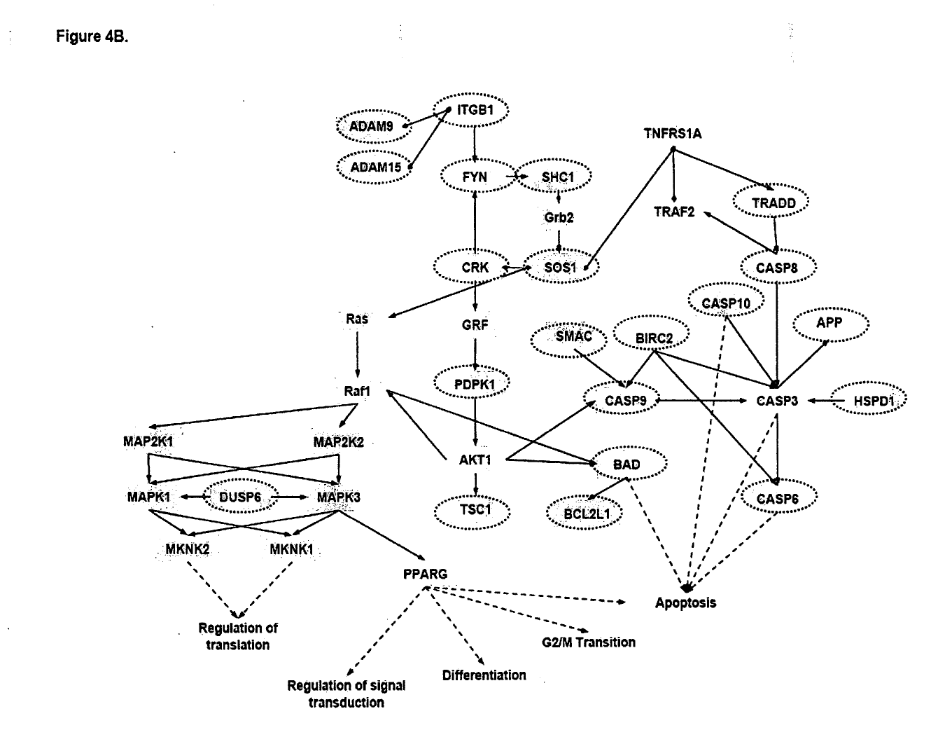

A number of genes were identified that were involved in the MAPK signaling pathway (e.g., ATF2, ATF4, DUSP6, DUSP10, IL1R2, MAP2K3, MAP4K3, MAPK14, MAX, MEF2A, PIK3R5, SOS1, SOS2 and TGFBR2) and in inflammatory response (e.g., ALOX5, CCL7, CCR2, CCR4, CD97, CD163, NFRKB, NLRP3, PLAA, SPN, TLR4, TLR6, TLR8), consistent with prior reports in the literature and the systemic pro-inflammatory characteristics associated with COPD (Mossman et al. 2006, American Journal of Respiratory Cell and Molecular Biology 34:666-669; Agusti et al. 2003, European Respiratory Journal 21:347-360; Rahman et al. 1996, American Journal of Respiratory and Critical Care Medicine 154:1055-1060; Chung 2001, European Respiratory Journal Supplement 34:50s-59s; Chung 2005, Curr Drug Targets Inflamm Allergy 4:619-625; Rahman 2005, Treatments in Respiratory Medicine 4:175-200; Agusti & Soriano 2008, Journal of Chronic Obstructive Pulmonary Disease 5:133-138; Fabbri & Rabe 2007, Lancet 370:797-799). A summary of the protein-protein interactions and possible biological outcomes identified by Pathway Studio from the list of candidate predictor genes is shown in FIG. 4.

L₁ Penalized Logistic Regression Model

In order to identify a more focused set of variables having a similar predictive capability as the random forest, an L₁ penalized logistic regression model was fit to predict the dichotomous outcome variable (Case/Control status) using the 1,014 variables identified by the random forest algorithm. L₁ penalized models are effective in performing automatic variable selection (Tibshirani, 1996). The model was first fit using data from the training set of 33 Cases and 34 Controls used to derive the random forest model. The final model, selected as the L₁ logistic regression model with minimum AIC (data not shown), comprised 9 predictor genes: IL6R, CCR2, PPP2CB, RASSF2, and WTAP were up-regulated and DNTTIP2, GDAP1, LIPE, and RPL14 were down-regulated in Cases compared with Controls. As shown in Table III, the 9-gene model had an overall error rate of 3.0%, discordantly classifying 1 spirometric Case and 1 spirometric Control. The derived L₁ penalized logistic regression model was subsequently applied to classify the test set of 70 subjects with FEV₁/PVC of 0.60-0.75, although one subject was excluded for missing a key variable leaving 69 subjects in the test set. The overall misclassification rate was 21.7% (Table III). The calculated sensitivity, specificity, and positive and negative predictive values in the test set of samples for both models are shown in Table IV.

TABLE III
Spirometric class versus L₁ penalized logistic regression
model-predicted class with associated
class-specific discordance rates for the training set
(FEV₁/FVC <0.60 or >0.75) and the test set (FEV₁/FVC 0.60-0.75).

| | Spirometric class | | | |
|---|---|---|---|---|
| | Training set | | Test set | |
| | (n = 67) | | (n = 69) | |
| | Cases | Controls | Cases | Controls |
| Predicted Class | | | | |
| Cases | 32 | 1 | 31 | 2 |
| Controls | 1 | 33 | 13 | 23 |
| Discordance | 3.0 | 2.9 | 29.5 | 8.0 |
| rate (%) | | | | |

FEV₁, forced expiratory volume in 1 s;
FVC, forced vital capacity

TABLE IV
Performance characteristics of the model-based classifiers in the test set
(n = 65, FEV₁/FVC 0.60-0.75).

| | Classifier Performance in Test Set | | | | | |
|---|---|---|---|---|---|---|
| | | Discordant | | | Positive | Negative |
| | Number of | Classification | Sensitivity | Specificity | Predictive | Predictive |
| Model Classifier | Variables | (%) | (%) | (%) | Value (%) | Value (%) |
| Full random forest | 1,014 | 24.6 | 65.9 | 91.7 | 93.1 | 61.1 |
| L₁-penalized logistic | 9 | 21.7 | 70.5 | 92.0 | 93.9 | 63.9 |
| regression | | | | | | |

FEV₁, forced expiratory volume in 1 s;
FVC, forced vital capacity

Biological Validation

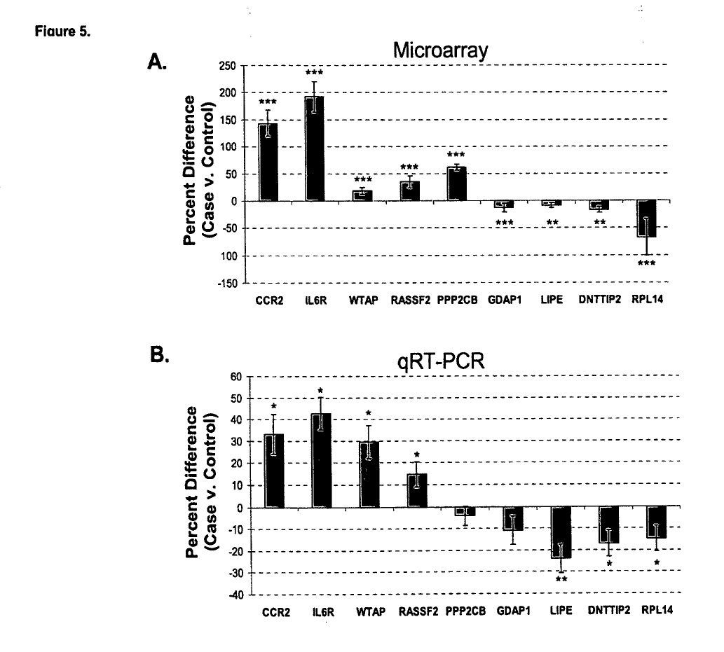

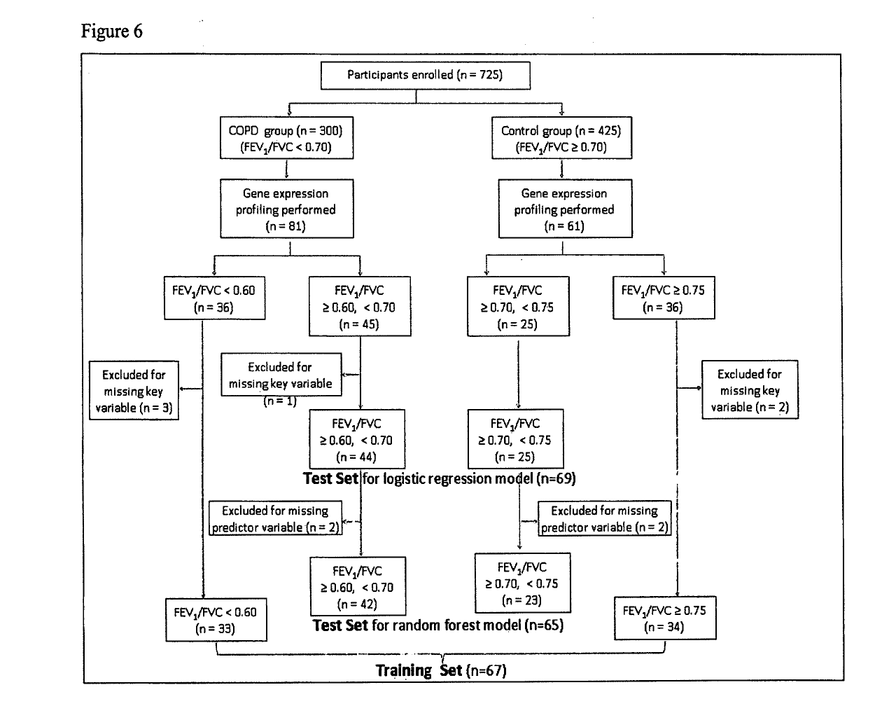

Real-time PCR was performed using isolated RNA from 24 randomly selected subjects in the training set (12 Cases and 12 Controls) to confirm the microarray results for the 9 predictor genes. Experimental results are shown in FIG. 5. Not all of the predictors from the microarray data were confirmed by qRT-PCR. However, a concordant directional trend in differential expression (Pearson correlation coefficient=0.795) between the two platforms for 7 of the 9 genes was observed, although in some instances the magnitude of the difference between Cases and Controls by qRT-PCR varied from that detected by microarray. No statistically significant differences were observed for PPP2CB and GDAP1 by qRT-PCR.

Using microarray analysis of PBL and random forest modeling, 1,013 genes were identified. One phenotypic variable was identified as a candidate predictor capable of differentiating smokers (current or former) with or without COPD. Gene ontology analyses indicate that these genes are involved in various cellular processes including regulation of apoptosis, regulation of cell growth, macromolecule (protein and RNA) transport, post-translational protein modification, cellular defense response, inflammatory response and RNA processing. A 9-gene subset derived from the larger set of candidate predictors that reliably discriminated between COPD and non-COPD objects was also identified. Differential expression of 7 of the 9 genes identified was confirmed by qRT-PCR, corroborating the microarray results.

The full random forest predictive model discordantly classified, or “misclassified,” 6% of the training set and 24.6% of the test set, and the 9-gene model differed from the spirometrically-defined classification for 3% of the training set and 21.7% of the test set. These models performed well in the more phenotypically extreme (by spirometry) training set and less well in the test set whose FEV₁/FVC values more closely bordered the diagnostic Case/Control cutoff value of 0.70. The great majority of the discordantly classified subjects in the test set were classified as Cases by spirometry but as Controls by their gene expression profile. It is possible for an individual to have a spuriously low airflow measurement that could result in a misdiagnosis of COPD by the GOLD guideline, which uses a fixed, arbitrary cutoff value of FEV₁/FVC.

Furthermore, although spirometric parameters are the traditional diagnostic and prognostic markers for COPD, it has become clear that they do not adequately represent all of its respiratory and systemic aspects (Marin et al. 2009, Respiratory Medicine 103(3):373-378; Celli 2006, Proceedings of the American Thoracic Society 3:461-465). FEV₁ correlates poorly with the degree of dyspnea, and the change in FEV₁ does not reflect the rate of decline in health status (Celli et al. 2004, Celli 2006, Burge et al. 2000, British Medical Journal 320:1297-1303). Other factors, such as emphysema and hyperinflation (Casanova et al. 2005, American Journal of Respiratory and Critical Care Medicine 171:591-597), malnutrition (Schols et al. 1998, American Journal of Respiratory and Critical Care Medicine 157:1791-1797), peripheral muscle dysfunction (Maltais et al. 2000, Clinics in Chest Medicine 21:665-677), and dyspnea (Nishimura et al. 2002, Chest 121:1434-1440), are independent predictors of outcome. In fact, the multifactorial BODE index that includes body mass index (B), degree of airflow obstruction (0), dyspnea score (D), and exercise endurance (E), is a better predictor of mortality than FEV₁ alone (Celli et al. 2004, The New England Journal of Medicine 350:1005-1012). The PBL gene expression profile alone or in combination with clinical markers such as the BODE components and/or lung parenchymal or airway changes on chest CT scans (Omori et al. 2006, Respirology 11:205-210) may be more predictive of the (early) presence, activity, and progression of the multi-component syndrome that is COPD than the clinical parameters alone.

One of the major constraints of COPD biomarker discovery has been the accessibility of suitable samples. In the past, sputum, bronchoalveolar lavage fluid, exhaled breath condensate, and bronchial biopsy tissue have been used (Sin & Man 2008, Chest 133:1296-1298). However, the sampling methodologies for such specimens are limited by their invasiveness and poor reproducibility. Since COPD is accompanied by systemic changes, as well as increased serum levels of certain proteins [e.g., C-reactive protein (CRP), interleukin 6 (IL-6), IL-8, leukotriene B₄ (LTB4), and TNFa], the use of PBLs as a surrogate biosample is an ideal alternative because they can be easily collected in large quantities at multiple time points using a relatively non-invasive procedure (Celli 2006; Schols et al. 1996, Thorax 51:819-824; Rahman & Biswas 2004, Redox Report: Communications in Free Radical Research 9:125-143; Rahman et al. 1996, Vemooy et al. 2002, American Journal of Respiratory and Critical Care Medicine 166:1218-1224; Agusti et al. 2003, Noguera et al. 1998, American Journal of Respiratory and Critical Care Medicine 158:1664-1668). As noted earlier, PBL gene expression profiles are successfully used to identify the presence or risk of other diseases having prominent systemic components.

Due to the role of PBLs in inflammation, the gene expression differences between subjects with and without COPD in this population of cells can reflect the degree of systemic inflammation or inflammation in the lungs. Lung inflammation is known to increase with the severity of the disease, as classified by the degree of airflow limitation (Hogg et al. 2004). The gene expression-based classifier is derived from the training set of COPD subjects with the most extreme airflow limitation, who likely also have the greatest degree of inflammation, while the test group with lesser airflow limitation may be predicted to have less inflammation. This may also partially account for the lower predictive ability between spirometric Cases and Controls in the test set compared to the training set.

In the present study, biological processes identified as over-represented in the set of COPD predictor genes include regulation of apoptosis, regulation of cell growth, macromolecule (protein and RNA) transport, post-translational protein modification, cellular defense response, inflammatory response and RNA processing. Major pathways identified include apoptosis, p38/MAPK signaling, focal adhesion, and leukocyte transendothelial migration. Changes in these biological processes and pathways may reflect the changes in activation, differentiation and cellular composition of the samples analyzed. The identification of leukocyte transendothelial migration is an important change in this cell population as COPD is characterized by leukocyte infiltration in the lung parenchyma (Panina et al. 2006, Current Drug Targets 7:669-674). Differences in expression of these genes may result in a predisposition of leukocyte subpopulations to infiltrate the lung tissue, and perhaps other tissues. This observation is supported by previously reported changes in chemotaxis and extracellular proteolysis in neutrophils isolated from the blood of subjects with COPD (Burnett et al. 1987, Lancet 2:1043-1046).

The subset of 9 genes identified using L₁ penalized logistic regression modeling have similar predictive performance as the full set of candidate predictors identified by the random forest model. It includes 5 up-regulated genes (CCR2, IL6R, PPP2CB, RASSF2, and WTAP) and 4 down-regulated genes (DNTTIP2, GDAP1, LIPE, RPL14) in COPD Cases compared with Controls. IL6R and CCR2 have been previously reported to have possible roles in COPD development and progression (Owen 2001, Pulmonary Pharmacology and Therapeutics 14:193-202; Wilk et al. 2007, BMC Medical Genetics 8 Suppl 1:S8). However, there have been no prior reports of an association with COPD for DNITIP2, GDAP1, LIPE, PPP2CB, RASSF2, RPL14 and WTAP.

The IL6R gene codes for the IL6 receptor, which is only reported to be expressed in subpopulations of leukocytes (monocytes, neutrophils and T and B lymphocytes) and hepatocytes (Chalaris et al. 2007, Blood 110:1748-1755; Jones et al. 2001, The FASEB Journal 15:43-58; Hamid et al. 2004, Diabetes 53:3342-3345). Many cell types do not express IL6R and are not directly responsive to IL6 (Chalaris et al. 2007, Jones et al. 2001). However, these cell types can be stimulated by IL6 bound to a soluble form of the IL6 receptor in a process called trans-signaling (Chalaris et al. 2007, Jones et al. 2001). IL6R shedding and subsequent release of the soluble form of the receptor results from cleavage of the membrane-bound receptor during apoptosis, a biological process and pathway identified in the gene expression signatures. This process is dependent on the metalloproteinases, ADAM17 and to a lesser extent ADAM10 (Chalaris et al. 2007, Matthews et al. 2003, The Journal of Biological Chemistry 278:38829-38839). ADAM17 was also found to be up-regulated in the microarray and was identified as one of the candidate predictor genes. Reported inducers of IL6R shedding include phorbol myristate acetate, cholesterol depletion, CRP, bacterial toxins, Fas stimulation and ultraviolet light (Chalaris et al. 2007, Mullberg et al. 1992, Biochemical and Biophysical Research Communications 189:794-800; Jones et al. 1999, Journal of Experimental Medicine 189:599-604; Matthews et al. 2003). Signaling through IL6R has also been shown to have a role in both inflammation and apoptosis (Finotto et al. 2007, Int Immunol 19:685-693). Furthermore, genome-wide association analyses have identified IL6R as a likely candidate gene for association with lung function (Wilk et al. 2007).

CCR2, which encodes the receptor for monocyte chemoattractant protein 1 and 3 (MCP1 and MCP3), is involved in inflammatory processes related to rheumatoid arthritis, alveolitis and tumor infiltration (Owen 2001). Higher levels of MCP1 mRNA and protein are detected in the bronchiolar epithelium in subjects with COPD, and increased levels of CCR2 are detected in macrophages, mast cells and epithelial cells of COPD subjects, indicating that MCP1 and CCR2 are involved in the recruitment of macrophages into the airway epithelium (Owen 2001, de Boer et al. 2000, Journal of Pathology 199:619-626). This increased expression of CCR2 also correlates with increased levels of mast cells and macrophages in the lungs of COPD subjects (de Boer et al. 2000). In addition, it has been demonstrated that activated neutrophils migrate in response to MCP1 (Johnston et al. 1999, The Journal of Clinical Investigation 103:1269-1276). These findings indicate mechanistic roles of IL6R and CCR2 in systemic and lung inflammation in COPD.

The 7 other genes in the 9-gene profile have varied biological functions. PPP2CB encodes the beta-isoform of the catalytic subunit of protein phosphatase 2A (PP2A) (Hemmings et al. 1988, Nucleic Acids Research 16:11366; Cohen 1989, Annual Review of Biochemistry 58:453-508). PP2A has been shown to regulate apoptosis in neutrophils by dephosphorylating both p38/MAPK and its substrate caspase 3, suggesting that PP2A has a role in the induction of apoptosis and the resolution of inflammation (Alvarado-Kristensson & Andersson 2005, The Journal of Biological Chemistry 280:6238-6244). RASSF2 promotes apoptosis and cell cycle arrest (Vos et al. 2003, The Journal of Biological Chemistry 278:28045-28051). WTAP is involved in the expression of genes related to cell division cycle and the G2/M checkpoint (Horiuchi et al. 2006, PNAS USA 103:17278-17283). The DNTT-interacting protein 2 (DNTTIP2), also known as estrogen receptor-binding protein, can bind the estrogen receptor-alpha and enhance its transcriptional activity in an estrogen-dependent manner (Bu et al. 2004, Biochemical and Biophysical Research Communications 317:54-59). GDAP1, or ganglioside-induced differentiation-associated protein 1, is found localized in the mitochondrial outer membrane and regulates the mitochondrial network. Over-expression of GDAP1 induces fragmentation of mitochondria without inducing apoptosis, affecting overall mitochondrial activity, or interfering with mitochondrial fusion (Niemann et al. 2005, The Journal of Cell Biology 170:1067-1078; Cuesta et al. 2002, Nature Genetics 30:22-25). LIPE, also know as HSL (hormone-sensitive lipase), has a role in the mobilization of free fatty acids from adipose tissue by controlling the rate of lipolysis of the stored triglycerides (Holm et al. 1988, Nucleic Acids Research 16:9879). Finally, RPL14 is a gene coding for a protein of the large ribosomal subunit (Robledo et al. 2008, RNA 14:1918-1929). The role of these genes in COPD may be linked to the cellular processes and pathways, such as cell cycle regulation and apoptosis, associated with the full list of genes.

Some factors, such as cellular composition of the sample, may influence the gene expression profiles detected by microarray in this study. Although the average total circulating WBC counts were similar between the groups with and without COPD, the mean lymphocyte and granulocyte counts as percentages of the total were significantly different (Table I). These parameters were included in the random forest analysis yet were not retained in the final model, indicating that the gene expression differences were more predictive of COPD status than lymphocyte and granulocyte percentages. Due to the random forest algorithm's inability to handle missing values among the predictor variables, the medication history of the subjects was not included in the analysis as several subjects had missing values. Although it is unclear how corticosteroids might affect gene expression in PBLs, it is known that the small airway inflammation responsible for airflow obstruction in COPD is poorly sensitive to the anti-inflammatory effects of corticosteroids (Hogg et al. 2004, The New England Journal of Medicine 350:2645-2653; Barnes 2006, Chest 129:151-155). Recent evidence has attributed this to oxidative and nitrative stress-induced reduction in histone deacetylase expression in inflammatory cells, thus preventing activated corticosteroid receptors from reversing the acetylation of activated inflammatory genes and turning off their transcription (Barnes 2006). Analysis of 10 subjects with possible indeterminate spirometric COPD Case/Control status based on their combination of FEV₁/FVC and FEV₁% predicted, categorizing them spirometrically as Controls by the GOLD-identified FEV₁/FVC cutoff value is also included. Only one of these subjects, in the test set, was discordantly classified as a Case by the gene expression profile (both the full and reduced models).

Cigarette smoke exposure can also influence gene expression, and of the 1,013 predictor genes identified in this analysis, differential expression of ATF4, MCL1, MAPK14, SERPINA1 and SOD2 was also identified in a study by van Leeuwen et al. (2007, Carcinogenesis 28:691-697), as strongly correlating with serum cotinine levels, a biomarker of recent exposure to tobacco. Two additional genes in the list, CCR2 and EPB41, are observed by Lampe et al. (2004, Cancer Epidemiology, Biomarkers & Prevention 13:445-453) as part of a cigarette smoke exposure molecular signature. Both the van Leeuwen and Lampe studies use PBLs isolated from current smokers and non-smokers indicating that the differential gene expression of some of the genes identified in this analysis may be related to tobacco smoke exposure. In a study of bronchial epithelial cells from never, current and former smokers, Beane et al. (2007, Genome Biology 8:R201) found 175 genes differentially expressed between never and current smokers, with irreversible changes in expression for 28 genes, slowly reversible for 6 genes and rapidly reversible for 139 genes. This indicates that duration and possibly intensity of cigarette smoking, and length of time since quitting, may be important confounding variables to gene expression analysis. The 1 phenotypic variable identified as a candidate predictor in this analysis (‘years of daily smoking’) appears to support this possibility.

This example indicates, among other things, that a training set and test set can be established that permit the identification of differential gene expression (1,013 genes in this instance) occurring in peripheral WBCs that discriminated between cigarette smokers with or without spirometrically defined COPD. The group of 1,013 genes can be reduced to a 9-gene subset with similar performance in differentiating smokers with or without COPD. Gene ontology and pathway analyses indicate that these genes are involved in regulation of apoptosis, regulation of cell growth, macromolecule (protein and RNA) transport, RNA processing, post-translational protein modification, cellular defense response, and inflammatory response. This is the first study to use microarray analysis of PBLs to identify gene expression differences associated with COPD. PBL samples are easy to obtain and their analysis complements current clinical diagnostic procedures for COPD. The gene expression profiles identified are novel biomarkers for COPD.

SUPPLEMENTARY TABLE I
Supplementary Table I. Phenotypic and smoking history
variables evaluated in random forest analysis.
Phenotypic variables included in random forest model
Gender
Age on spirometry test date
Age when first tried a cigarette
Age when first started smoking daily
Years of daily smoking
Pack-years of smoking
Current smoking status
Average number of cigarettes per day during past 3 months
Whether currently smoking ≧1 cigarettes on most days
Height (cm)
Weight (kg)
Body mass index [kg (m²)⁻¹]
Systolic blood pressure (mm Hg)
Diastolic blood pressure (mm Hg)
Blood hemoglobin concentration (g dL⁻¹)
Blood hematocrit (%)
Total white blood cell count (WBC, 10³ μL⁻¹)
Blood basophils as % of total WBC
Blood eosinophils as % of total WBC
Blood granulocytes as % of total WBC
Blood lymphocytes as % of total WBC
Blood monocytes as % of total WBC
Carboxyhemoglobin concentration (% saturation)

### Supplementary TableII

Unless otherwise indicated, the nucleic acids listed or set forth in Supplementary Table II include: nucleic acids having the sequences recited in the table and/or their complement; the sequences of nucleic acids transcribed from the genes or loci listed in the table or their complement; and either or both strands (if double stranded) of cDNAs clones of the nucleic acids transcribed from the genes or loci listed in the table. The nucleic acids listed or set forth in Supplementary Table II also include the specific nucleic acid sequences listed under the NCBI accession and/or the NCBI GI number categories and their complementary sequences.

SUPPLEMENTARY TABLE II
Complete list of covariates identified as having significant Gini variable importance measures by random
forest modeling, with the fold change between cases and controls along with the 95% lower confidence (lcl)
and upper confidence limits (ucl). For the variable included that was not a gene (years daily smoking)
the average number of more years daily smoking and its confidence interval are reported rather than fold change.

| | | | | Illumina | | | | |
|---|---|---|---|---|---|---|---|---|
| Covariate | NCBI Accession | NCBI GI | Illumina | Array | | Fold | | |
| (Gene Name) | and Version* | Number* | Search Key | Address ID | Gini | Change | lcl | ucl |
| ASAH1 | NM_177924.1 | 30089927 | ILMN_26236 | 840161 | 0.1619 | 2.32 | 1.99 | 2.67 |
| CD97 | NM_078481.2 | 68508935 | ILMN_26363 | 1400121 | 0.1324 | 2.52 | 2.14 | 2.93 |
| LOC653518 | XM_930277.1 | 88961606 | ILMN_35449 | 3800082 | 0.1292 | 3.55 | 2.88 | 4.20 |
| (CCR2) | | | | | | | | |
| PPP2CB | NM_001009552.1 | 57222564 | ILMN_22922 | 4390446 | 0.1248 | 1.65 | 1.53 | 1.78 |
| CTBP2 | NM_001329.1 | 4557498 | ILMN_20261 | 4230736 | 0.1227 | 2.07 | 1.86 | 2.30 |
| SAMHD1 | NM_015474.2 | 38016913 | ILMN_17752 | 7320047 | 0.1187 | 2.13 | 1.89 | 2.37 |
| LOC653723 | XM_929209.1 | 89056911 | ILMN_46534 | 4120673 | 0.1179 | 1.70 | 1.56 | 1.86 |
| LOC644584 | XM_927700.1 | 89037308 | ILMN_38997 | 7040739 | 0.1156 | 2.28 | 2.01 | 2.55 |
| CCNC | NM_005190.3 | 61676090 | ILMN_11667 | 7210121 | 0.1137 | 2.57 | 2.17 | 3.01 |
| LOC653105 | XM_931214.1 | 88944406 | ILMN_44054 | 3800139 | 0.1108 | 1.63 | 1.50 | 1.76 |
| ACOX1 | NM_004035.4 | 34304338 | ILMN_138201 | 3450138 | 0.1086 | 1.69 | 1.53 | 1.85 |
| PGAM1 | NM_002629.2 | 31543395 | ILMN_26357 | 6220242 | 0.1075 | 3.97 | 3.18 | 4.84 |
| LILRA1 | NM_006863.1 | 5803065 | ILMN_2616 | 6660400 | 0.1061 | 1.56 | 1.43 | 1.69 |
| IL6R | NM_000565.2 | 31317250 | ILMN_22419 | 6250360 | 0.104 | 3.63 | 3.04 | 4.29 |
| LOC653994 | XM_944429.1 | 89026095 | ILMN_38337 | 3290470 | 0.1035 | 6.81 | 5.11 | 8.65 |
| LOC645508 | XM_928532.1 | 89025625 | ILMN_38571 | 5270164 | 0.1027 | 1.53 | 1.43 | 1.63 |
| CSF2RB | NM_000395.1 | 4559407 | ILMN_5898 | 6330079 | 0.1006 | 2.32 | 2.00 | 2.69 |
| SNX5 | NM_152227.1 | 23111046 | ILMN_6733 | 1030424 | 0.0997 | 1.83 | 1.63 | 2.06 |
| MAGED2 | NM_014599.4 | 29171703 | ILMN_17148 | 380025 | 0.0988 | 1.48 | 1.38 | 1.58 |
| SLC37A3 | NM_207113.1 | 46361975 | ILMN_15540 | 6380575 | 0.0969 | 1.60 | 1.44 | 1.77 |
| ASB7 | NM_198243.1 | 38176282 | ILMN_13798 | 50750 | 0.0965 | 1.44 | 1.35 | 1.53 |
| PTPNS1L3 | XM_944363.1 | 89057937 | ILMN_30873 | 3990544 | 0.0964 | 1.42 | 1.31 | 1.53 |
| C14ORF150 | NM_080666.2 | 57165357 | ILMN_1497 | 5220369 | 0.096 | 1.29 | 1.22 | 1.36 |
| ALDOA | NM_184041.1 | 34577109 | ILMN_19652 | 4590671 | 0.0953 | 4.64 | 3.49 | 6.13 |
| MME | NM_000902.2 | 6042205 | ILMN_21688 | 2360400 | 0.0943 | 1.49 | 1.36 | 1.63 |
| KLF10 | NM_005655.1 | 5032176 | ILMN_2466 | 5810280 | 0.0941 | 2.45 | 2.11 | 2.82 |
| LOC651348 | XM_946163.1 | 89057480 | ILMN_32410 | 6480619 | 0.094 | 3.96 | 3.17 | 4.90 |
| MLKL | NM_152649.1 | 22749322 | ILMN_25241 | 6960204 | 0.0928 | 2.45 | 2.08 | 2.86 |
| HNRPM | NM_005968.2 | 14141151 | ILMN_24927 | 2070309 | 0.0921 | 1.38 | 1.29 | 1.46 |
| IL6R | NM_181359.1 | 31317248 | ILMN_6641 | 2600475 | 0.0918 | 3.39 | 2.78 | 4.11 |
| C1ORF108 | NM_024595.1 | 13375790 | ILMN_6070 | 2640243 | 0.0911 | 1.90 | 1.70 | 2.09 |
| KIAA0251 | NM_015027.1 | 39930344 | ILMN_14287 | 4290274 | 0.0907 | 1.60 | 1.49 | 1.72 |
| CECR1 | NM_017424.2 | 29029549 | ILMN_10713 | 650592 | 0.0899 | 3.13 | 2.56 | 3.73 |
| LOC653738 | XM_929341.1 | 88961756 | ILMN_37845 | 2370019 | 0.0892 | 1.41 | 1.31 | 1.50 |
| GLUL | NM_001033056.1 | 74271825 | ILMN_26367 | 670537 | 0.089 | 2.67 | 2.29 | 3.06 |
| TUBB | NM_178014.2 | 34222261 | ILMN_23399 | 1580484 | 0.0872 | 2.50 | 2.10 | 2.90 |
| MATR3 | NM_018834.4 | 62750352 | ILMN_15182 | 4810577 | 0.0862 | 2.02 | 1.78 | 2.27 |
| SON | NM_138926.1 | 21040321 | ILMN_12440 | 2940435 | 0.086 | 1.40 | 1.32 | 1.48 |
| LOC648763 | XM_940246.1 | 88979438 | ILMN_30575 | 3180349 | 0.0854 | 2.83 | 2.33 | 3.39 |
| ACTG1 | NM_001614.2 | 11038618 | ILMN_24353 | 6520497 | 0.0851 | 6.23 | 4.43 | 8.63 |
| DDX19B | NM_001014451.1 | 62241023 | ILMN_17268 | 7210471 | 0.085 | 1.39 | 1.30 | 1.47 |
| SRP54 | XM_940545.1 | 89037651 | ILMN_138804 | 7380221 | 0.0849 | 1.82 | 1.63 | 2.00 |
| GPR97 | NM_170776.3 | 40538803 | ILMN_18651 | 6110630 | 0.0848 | 3.31 | 2.68 | 4.01 |
| UTRN | NM_007124.1 | 6005937 | ILMN_15375 | 4570470 | 0.0845 | 1.75 | 1.59 | 1.92 |
| LOC644330 | XM_934365.1 | 89056804 | ILMN_42347 | 1430079 | 0.0844 | 4.26 | 3.36 | 5.34 |
| ARFIP1 | NM_001025595.1 | 71040093 | ILMN_16086 | 1430364 | 0.0839 | 1.85 | 1.67 | 2.06 |
| NBR1 | NM_005899.2 | 14110374 | ILMN_16223 | 2970324 | 0.0825 | 2.01 | 1.75 | 2.27 |
| LOC653094 | XM_925947.1 | 89059738 | ILMN_35175 | 1070128 | 0.0823 | 1.67 | 1.53 | 1.81 |
| LOC644063 | XM_931572.1 | 88965390 | ILMN_40116 | 6520639 | 0.0814 | 6.71 | 4.87 | 9.10 |
| C10ORF46 | NM_153810.3 | 54262140 | ILMN_14628 | 2070286 | 0.0801 | 1.88 | 1.67 | 2.10 |
| LOC653895 | XM_936379.1 | 89033487 | ILMN_38756 | 1440273 | 0.08 | 1.26 | 1.20 | 1.32 |
| LOC647474 | XM_943003.1 | 89061094 | ILMN_42643 | 6480465 | 0.0795 | 1.46 | 1.37 | 1.56 |
| LBH | NM_030915.1 | 13569871 | ILMN_21350 | 150592 | 0.0791 | 1.82 | 1.61 | 2.04 |
| CSTF1 | NM_001033521.1 | 75709216 | ILMN_28771 | 3520634 | 0.0786 | 1.40 | 1.32 | 1.49 |
| LSM12 | NM_152344.1 | 22748746 | ILMN_1510 | 3990338 | 0.0784 | 2.06 | 1.82 | 2.32 |
| RASSF1 | NM_170712.1 | 25777679 | ILMN_11841 | 1820470 | 0.0783 | 1.25 | 1.20 | 1.30 |
| LOC650667 | XM_939756.1 | 89059311 | ILMN_36687 | 60711 | 0.078 | 1.79 | 1.62 | 1.95 |
| HS.571253 | DA938875 | 82424570 | ILMN_123434 | 3140414 | 0.0776 | 1.73 | 1.55 | 1.90 |
| LOC646144 | XM_935294.1 | 89025359 | ILMN_45775 | 3120671 | 0.0775 | 1.43 | 1.32 | 1.53 |
| MARCH1 | NM_017923.2 | 53759068 | ILMN_30212 | 1070326 | 0.0771 | 2.60 | 2.17 | 3.07 |
| CDC42 | NM_044472.1 | 16357471 | ILMN_137677 | 1030035 | 0.0766 | 2.82 | 2.30 | 3.41 |
| WAC | NM_100264.1 | 18379329 | ILMN_28064 | 6100136 | 0.076 | 2.05 | 1.81 | 2.28 |
| LOC652388 | XM_941821.1 | 89071419 | ILMN_45950 | 7320259 | 0.076 | 1.96 | 1.73 | 2.21 |
| CRTAP | NM_006371.3 | 53759127 | ILMN_2952 | 1470044 | 0.0759 | 3.64 | 2.88 | 4.51 |
| TNPO1 | NM_153188.1 | 23510380 | ILMN_29083 | 1570397 | 0.0759 | 1.68 | 1.54 | 1.82 |
| CRK | NM_016823.2 | 41327711 | ILMN_25875 | 5810176 | 0.0758 | 2.01 | 1.78 | 2.25 |
| ALOX5 | NM_000698.2 | 62912458 | ILMN_2997 | 6220097 | 0.0751 | 1.36 | 1.28 | 1.44 |
| LOC646309 | XM_929247.1 | 89030887 | ILMN_44679 | 4390246 | 0.0749 | 1.53 | 1.42 | 1.65 |
| FBXO7 | NM_012179.3 | 74229026 | ILMN_28542 | 1690070 | 0.0744 | 2.03 | 1.80 | 2.27 |
| LYPLA1 | NM_006330.2 | 20302148 | ILMN_5453 | 2070673 | 0.0744 | 2.56 | 2.16 | 3.00 |
| KUA-UEV | NM_199203.1 | 40806189 | ILMN_20084 | 4540561 | 0.074 | 1.61 | 1.47 | 1.75 |
| WSB1 | NM_134264.2 | 58331182 | ILMN_674 | 5260673 | 0.074 | 2.12 | 1.84 | 2.43 |
| LOC653491 | XM_927709.1 | 89025111 | ILMN_37211 | 1170646 | 0.0737 | 1.66 | 1.51 | 1.82 |
| C20ORF14 | NM_012469.2 | 40807484 | ILMN_18026 | 4180670 | 0.0737 | 2.19 | 1.93 | 2.47 |
| LOC389850 | XM_372205.4 | 89059568 | ILMN_45804 | 2900619 | 0.0732 | 1.50 | 1.38 | 1.63 |
| MAEA | NM_001017405.1 | 62953130 | ILMN_4828 | 6660470 | 0.0732 | 2.83 | 2.41 | 3.31 |
| SLIC1 | NM_182854.1 | 33504570 | ILMN_511 | 3710767 | 0.0729 | 2.03 | 1.76 | 2.30 |
| ACSL5 | NM_016234.3 | 42794755 | ILMN_6741 | 4010619 | 0.0724 | 1.39 | 1.30 | 1.47 |
| GRAP | NM_006613.3 | 50659102 | ILMN_5687 | 110703 | 0.072 | 1.53 | 1.39 | 1.68 |
| NOMO2 | NM_173614.2 | 51944972 | ILMN_1736 | 60717 | 0.072 | 1.63 | 1.45 | 1.80 |
| LOC651106 | XM_940235.1 | 89061862 | ILMN_44908 | 2570014 | 0.0718 | 1.49 | 1.39 | 1.61 |
| ZDHHC13 | NM_019028.2 | 47933345 | ILMN_24550 | 4250592 | 0.0716 | 1.26 | 1.20 | 1.31 |
| ECD | NM_007265.1 | 6005783 | ILMN_25476 | 2120379 | 0.0714 | 2.16 | 1.88 | 2.48 |
| MPEG1 | XM_166227.6 | 89033974 | ILMN_38016 | 7380008 | 0.0709 | 4.19 | 3.32 | 5.11 |
| WDFY3 | NM_014991.3 | 31317271 | ILMN_12455 | 4260280 | 0.0708 | 1.57 | 1.44 | 1.71 |
| SPG21 | XM_945608.1 | 89039020 | ILMN_137401 | 4260195 | 0.0704 | 2.94 | 2.41 | 3.59 |
| RASSF2 | NM_170773.1 | 25777674 | ILMN_137091 | 4570333 | 0.0704 | 1.31 | 1.24 | 1.37 |
| CDV3 | NM_017548.3 | 52856418 | ILMN_11989 | 4860386 | 0.0703 | 1.37 | 1.28 | 1.46 |
| SLC3A2 | NM_001013251.1 | 61744482 | ILMN_12826 | 4280458 | 0.07 | 2.42 | 2.11 | 2.74 |
| NIPA2 | NM_030922.5 | 57013273 | ILMN_3795 | 5270682 | 0.0698 | 1.48 | 1.38 | 1.58 |
| TFG | NM_006070.4 | 56090655 | ILMN_7895 | 6520180 | 0.0698 | 1.48 | 1.37 | 1.58 |
| LOC654189 | XM_942687.1 | 88968995 | ILMN_30702 | 7100386 | 0.0698 | 1.61 | 1.48 | 1.76 |
| ELMO1 | NM_014800.8 | 18765699 | ILMN_137709 | 4880133 | 0.0696 | 1.54 | 1.41 | 1.67 |
| FLJ25037 | XM_941208.1 | 89067009 | ILMN_137053 | 1570376 | 0.0694 | 1.44 | 1.34 | 1.55 |
| MAP2K3 | XM_944206.1 | 89042496 | ILMN_137034 | 4640131 | 0.0692 | 2.65 | 2.28 | 3.05 |
| TPM3 | NM_153649.2 | 39725631 | ILMN_17262 | 6590730 | 0.069 | 3.73 | 2.97 | 4.67 |
| PDLIM5 | NM_006457.2 | 58533152 | ILMN_12134 | 4480484 | 0.0688 | 1.51 | 1.40 | 1.62 |
| ST3GAL1 | NM_003033.2 | 27765097 | ILMN_2099 | 3370292 | 0.0686 | 1.91 | 1.71 | 2.12 |
| ARHGAP25 | NM_001007231.1 | 55770897 | ILMN_1674 | 4850079 | 0.0685 | 1.80 | 1.63 | 1.97 |
| LOC653133 | XM_926881.1 | 89024662 | ILMN_138087 | 2900288 | 0.0682 | 1.70 | 1.54 | 1.88 |
| KUA-UEV | NM_199203.1 | 40806189 | ILMN_20084 | 6280270 | 0.0677 | 1.75 | 1.58 | 1.92 |
| MDM4 | NM_002393.1 | 4505138 | ILMN_137381 | 4490671 | 0.0676 | 2.46 | 2.12 | 2.85 |
| HS.105636 | BX417162 | 46930487 | ILMN_74929 | 5220014 | 0.0675 | 1.99 | 1.76 | 2.22 |
| VASP | NM_003370.3 | 57165437 | ILMN_28263 | 5260161 | 0.0674 | 2.08 | 1.84 | 2.31 |
| NUP98 | NM_016320.3 | 56550110 | ILMN_21954 | 7650669 | 0.0668 | 1.64 | 1.49 | 1.80 |
| PICALM | NM_007166.2 | 56788365 | ILMN_23418 | 1580364 | 0.0665 | 2.63 | 2.20 | 3.09 |
| GGT2 | NM_002058.1 | 62079286 | ILMN_3296 | 4590523 | 0.0665 | 1.58 | 1.46 | 1.72 |
| LOC648189 | XM_937239.1 | 89039190 | ILMN_40837 | 1980059 | 0.0663 | 1.48 | 1.37 | 1.58 |
| GPR141 | NM_181791.1 | 32401434 | ILMN_20517 | 2260672 | 0.066 | 1.57 | 1.42 | 1.71 |
| BTN2A1 | NM_078476.1 | 17975771 | ILMN_28434 | 7210379 | 0.0656 | 1.73 | 1.56 | 1.90 |
| NEK7 | NM_133494.1 | 19424131 | ILMN_23490 | 4880553 | 0.0653 | 2.42 | 2.04 | 2.80 |
| LBR | NM_002296.2 | 37595749 | ILMN_7414 | 2360731 | 0.0649 | 5.61 | 4.31 | 7.10 |
| RPL14 | NM_003973.2 | 16753224 | ILMN_138835 | 3800280 | 0.0641 | −2.73 | −3.24 | −2.26 |
| UNC93B1 | NM_030930.2 | 45580708 | ILMN_8587 | 4560370 | 0.0641 | 2.46 | 2.10 | 2.84 |
| TM2D3 | NM_078474.1 | 17865799 | ILMN_28191 | 940273 | 0.0639 | 1.57 | 1.42 | 1.71 |
| GRINL1A | NM_001018102.1 | 70166831 | ILMN_20762 | 2850343 | 0.0637 | 1.79 | 1.60 | 1.99 |
| MLKL | XM_936963.1 | 89041041 | ILMN_139138 | 6350274 | 0.0637 | 2.60 | 2.18 | 3.07 |
| SETD3 | NM_199123.1 | 40068482 | ILMN_27724 | 2570035 | 0.0636 | 1.74 | 1.56 | 1.92 |
| SS18 | NM_001007559.1 | 56117845 | ILMN_22307 | 3890047 | 0.0635 | 1.25 | 1.18 | 1.32 |
| HFE | NM_139007.1 | 21040348 | ILMN_21360 | 2000487 | 0.0631 | 1.24 | 1.18 | 1.28 |
| LOC653383 | XM_927177.1 | 89030160 | ILMN_35816 | 2120521 | 0.0628 | 2.03 | 1.77 | 2.31 |
| MAPK14 | NM_139013.1 | 20986513 | ILMN_17267 | 6860717 | 0.0628 | 3.31 | 2.68 | 4.00 |
| FASTK | NM_006712.3 | 39995105 | ILMN_11299 | 650753 | 0.0626 | 1.81 | 1.63 | 2.00 |
| MRRF | NM_199176.1 | 40317621 | ILMN_4576 | 7380736 | 0.0625 | 1.33 | 1.24 | 1.41 |
| MAP2K3 | NM_145110.1 | 21618350 | ILMN_10112 | 4290524 | 0.0624 | 2.17 | 1.92 | 2.44 |
| MCRS1 | NM_006337.3 | 34222264 | ILMN_9875 | 5570445 | 0.0623 | 2.10 | 1.82 | 2.39 |
| NCOA2 | NM_006540.2 | 76253684 | ILMN_1913 | 4780039 | 0.0622 | 1.62 | 1.47 | 1.77 |
| EGFL5 | XM_929502.1 | 89029942 | ILMN_37703 | 7560615 | 0.0622 | 1.73 | 1.54 | 1.94 |
| WBSCR1 | NM_022170.1 | 11559922 | ILMN_6141 | 4540047 | 0.0614 | 1.57 | 1.41 | 1.73 |
| GTF2I | XM_939506.1 | 89026111 | ILMN_138994 | 3830348 | 0.0611 | 2.22 | 1.87 | 2.57 |
| NSF | XM_938198.1 | 89042742 | ILMN_136981 | 160735 | 0.0606 | 1.99 | 1.76 | 2.21 |
| NSF | NM_006178.1 | 11079227 | ILMN_23282 | 3830040 | 0.0605 | 2.04 | 1.79 | 2.30 |
| TSC22D3 | NM_198057.2 | 62865623 | ILMN_23548 | 1740327 | 0.0602 | 1.49 | 1.38 | 1.59 |
| MCFP | NM_018843.2 | 46094064 | ILMN_15963 | 6510326 | 0.0602 | 1.67 | 1.50 | 1.83 |
| CREB5 | NM_001011666.1 | 59938775 | ILMN_19827 | 4220026 | 0.06 | 2.70 | 2.20 | 3.27 |
| C1ORF183 | NM_019099.3 | 39545578 | ILMN_9599 | 6280431 | 0.0599 | 1.66 | 1.51 | 1.82 |
| PSEN1 | NM_000021.2 | 21536454 | ILMN_28849 | 6220754 | 0.0598 | 1.91 | 1.72 | 2.11 |
| RASSF5 | NM_182664.1 | 32996732 | ILMN_690 | 7560563 | 0.0596 | 3.55 | 2.92 | 4.30 |
| LOC648394 | XM_942936.1 | 89066728 | ILMN_33220 | 2630601 | 0.0593 | 2.01 | 1.75 | 2.32 |
| WDR1 | NM_017491.3 | 53729350 | ILMN_14280 | 3610767 | 0.059 | 5.09 | 4.01 | 6.39 |
| TCF20 | NM_181492.1 | 31652241 | ILMN_25080 | 3450093 | 0.0587 | 1.30 | 1.24 | 1.37 |
| MGC15875 | NM_153373.1 | 24119276 | ILMN_28180 | 7320288 | 0.0587 | 1.85 | 1.64 | 2.05 |
| DPP7 | NM_013379.2 | 62420887 | ILMN_6361 | 110274 | 0.0585 | 2.07 | 1.82 | 2.34 |
| ABCC1 | NM_019900.1 | 9955955 | ILMN_12532 | 2480543 | 0.0585 | 1.41 | 1.32 | 1.50 |
| CEPT1 | NM_006090.3 | 56119170 | ILMN_14637 | 7380441 | 0.0583 | 1.75 | 1.58 | 1.91 |
| USP4 | NM_003363.2 | 40795664 | ILMN_5953 | 3060709 | 0.0582 | 2.66 | 2.26 | 3.09 |
| SON | NM_032195.1 | 21040313 | ILMN_8462 | 6450128 | 0.058 | 2.33 | 2.02 | 2.66 |
| ADAM9 | NM_003816.2 | 54292119 | ILMN_922 | 7550082 | 0.0578 | 1.24 | 1.18 | 1.29 |
| OAS2 | NM_016817.2 | 74229018 | ILMN_5994 | 150056 | 0.0573 | 1.82 | 1.61 | 2.02 |
| ATF4 | NM_001675.2 | 33469975 | ILMN_10757 | 2900170 | 0.0572 | 1.68 | 1.51 | 1.87 |
| USP22 | XM_942262.1 | 89042515 | ILMN_38059 | 6560438 | 0.0569 | 1.90 | 1.69 | 2.12 |
| PBEF1 | NM_182790.1 | 33386694 | ILMN_13867 | 2690068 | 0.0567 | 6.05 | 4.36 | 8.24 |
| STK24 | NM_001032296.1 | 73808091 | ILMN_10104 | 4850373 | 0.0567 | 1.60 | 1.46 | 1.75 |
| C19ORF6 | NM_001033026.1 | 74229024 | ILMN_12941 | 3930064 | 0.0564 | 2.04 | 1.82 | 2.27 |
| TXNDC5 | NM_030810.2 | 42794770 | ILMN_24968 | 2900458 | 0.0553 | 1.55 | 1.39 | 1.72 |
| MAX | NM_197957.2 | 59814750 | ILMN_1660 | 6860682 | 0.055 | 2.08 | 1.80 | 2.40 |
| ERGIC1 | NM_001031711.1 | 72534711 | ILMN_7272 | 6060333 | 0.0549 | 2.31 | 1.97 | 2.68 |
| CLSTN1 | XM_937951.1 | 88945307 | ILMN_136995 | 270372 | 0.0544 | 1.29 | 1.22 | 1.36 |
| DPH2 | NM_001384.3 | 41352701 | ILMN_137484 | 670450 | 0.0539 | 1.37 | 1.28 | 1.45 |
| CTBP1 | NM_001012614.1 | 61743966 | ILMN_21952 | 6770113 | 0.0539 | 2.17 | 1.90 | 2.43 |
| CDK5RAP3 | NM_176095.1 | 28872789 | ILMN_11403 | 2940722 | 0.0538 | 3.14 | 2.59 | 3.65 |
| CDK2 | NM_001798.2 | 16936527 | ILMN_12332 | 450315 | 0.0537 | 1.45 | 1.35 | 1.54 |
| LOC344620 | XM_937279.1 | 88970732 | ILMN_35635 | 6100168 | 0.0536 | 2.33 | 2.03 | 2.64 |
| ELMO2 | NM_022086.6 | 33469944 | ILMN_19511 | 160403 | 0.0535 | 1.73 | 1.56 | 1.90 |
| DPAGT1 | NM_001382.2 | 42794008 | ILMN_10306 | 1990347 | 0.0534 | 1.75 | 1.56 | 1.93 |
| C9ORF72 | NM_145005.3 | 37039614 | ILMN_9580 | 840242 | 0.0534 | 2.31 | 1.96 | 2.70 |
| PHF12 | NM_020889.2 | 75677337 | ILMN_8914 | 1470025 | 0.0533 | 1.28 | 1.22 | 1.35 |
| RNF187 | XM_047499.9 | 88943868 | ILMN_37839 | 1440504 | 0.0531 | 1.28 | 1.21 | 1.33 |
| MAT2B | NM_013283.3 | 33519456 | ILMN_18923 | 5080494 | 0.0531 | 3.60 | 2.80 | 4.57 |
| LOC654174 | XM_940438.1 | 88999456 | ILMN_44671 | 1260112 | 0.0529 | 2.23 | 1.94 | 2.52 |
| VPS13C | NM_018080.2 | 66348090 | ILMN_2446 | 5890136 | 0.0529 | 1.48 | 1.39 | 1.58 |
| LOC652626 | XM_942172.1 | 89073794 | ILMN_44442 | 10274 | 0.0527 | 1.62 | 1.48 | 1.79 |
| TOP1MT | NM_052963.1 | 16418460 | ILMN_15321 | 1940594 | 0.0521 | 1.59 | 1.40 | 1.77 |
| DGKA | NM_001345.4 | 41393585 | ILMN_4980 | 4670021 | 0.052 | 1.42 | 1.31 | 1.54 |
| CTNNB1 | NM_001904.2 | 40254459 | ILMN_21386 | 6040201 | 0.0516 | 2.18 | 1.90 | 2.50 |
| HSPD1 | NM_002156.4 | 41399283 | ILMN_7269 | 940767 | 0.0513 | 1.34 | 1.26 | 1.41 |
| RNF135 | NM_032322.3 | 37655166 | ILMN_26639 | 3370041 | 0.0507 | 1.97 | 1.75 | 2.20 |
| TRUB1 | NM_139169.3 | 34303921 | ILMN_26216 | 4570215 | 0.0507 | 1.33 | 1.24 | 1.42 |
| HM13 | NM_030789.2 | 30581114 | ILMN_2780 | 3370326 | 0.0505 | 2.93 | 2.42 | 3.48 |
| MGAT4B | NM_014275.2 | 16915933 | ILMN_139177 | 1090328 | 0.0495 | 1.59 | 1.45 | 1.73 |
| RAE1 | NM_003610.3 | 62739174 | ILMN_24358 | 4010519 | 0.0492 | 1.69 | 1.53 | 1.84 |
| RAB37 | NM_001006638.1 | 54859684 | ILMN_8592 | 6940551 | 0.0492 | 3.16 | 2.61 | 3.78 |
| TAP2 | NM_018833.2 | 73747916 | ILMN_437 | 1780528 | 0.0491 | 1.81 | 1.58 | 2.10 |
| ACTB | NM_001101.2 | 5016088 | ILMN_2565 | 2650079 | 0.0478 | 3.10 | 2.49 | 3.80 |
| CPNE1 | NM_003915.2 | 23397694 | ILMN_22052 | 6520577 | 0.0478 | 1.80 | 1.62 | 1.99 |
| TPST2 | NM_003595.3 | 56699462 | ILMN_13359 | 620014 | 0.0477 | 1.77 | 1.59 | 1.96 |
| MRE11A | NM_005590.3 | 56550106 | ILMN_6718 | 2030762 | 0.0472 | 1.32 | 1.25 | 1.40 |
| CTGLF1 | NM_133446.1 | 19263342 | ILMN_22934 | 7000437 | 0.0472 | 1.88 | 1.68 | 2.07 |
| NFX1 | NM_147133.1 | 22212924 | ILMN_17577 | 5900338 | 0.0469 | 1.61 | 1.45 | 1.77 |
| LOC652878 | XM_942594.1 | 89065158 | ILMN_41407 | 6040634 | 0.0469 | 3.35 | 2.63 | 4.19 |
| LOC653518 | XM_934555.1 | 88961609 | ILMN_35512 | 270242 | 0.0467 | 3.02 | 2.46 | 3.65 |
| LOC441511 | XM_497141.2 | 89059964 | ILMN_41472 | 1980367 | 0.0466 | 1.66 | 1.50 | 1.82 |
| PTGS1 | NM_000962.2 | 18104966 | ILMN_24170 | 4060438 | 0.0463 | 1.59 | 1.46 | 1.73 |
| VNN2 | NM_078488.1 | 17865815 | ILMN_24337 | 2690079 | 0.0461 | 1.84 | 1.62 | 2.07 |
| GPR97 | XM_936582.1 | 89065470 | ILMN_138901 | 2690338 | 0.0461 | 3.61 | 2.86 | 4.42 |
| B3GNT1 | NM_006577.3 | 15451893 | ILMN_138549 | 4610082 | 0.0461 | 1.82 | 1.62 | 2.05 |
| DDB1 | XM_943551.1 | 89034785 | ILMN_139085 | 2690300 | 0.046 | 1.50 | 1.37 | 1.61 |
| FBXO9 | NM_033480.1 | 15812200 | ILMN_26635 | 2190129 | 0.0459 | 1.59 | 1.46 | 1.74 |
| GIMAP6 | NM_024711.3 | 56119213 | ILMN_1753 | 730327 | 0.0459 | 1.25 | 1.19 | 1.31 |
| FAM21C | NM_015262.1 | 59814410 | ILMN_17686 | 4890519 | 0.0457 | 1.92 | 1.71 | 2.15 |
| TES | NM_015641.2 | 23238186 | ILMN_17251 | 780524 | 0.0457 | 1.65 | 1.51 | 1.78 |
| TCIRG1 | NM_006019.2 | 19924144 | ILMN_2161 | 3850128 | 0.0455 | 2.47 | 2.06 | 2.93 |
| STOM | NM_004099.4 | 38016910 | ILMN_17469 | 4640484 | 0.0455 | 3.16 | 2.52 | 3.90 |
| ARHGAP30 | NM_001025598.1 | 71040097 | ILMN_15952 | 830189 | 0.0454 | 3.05 | 2.51 | 3.65 |
| LOC647481 | XM_936545.1 | 88952403 | ILMN_40110 | 540154 | 0.0453 | 1.63 | 1.47 | 1.78 |
| DHX9 | NM_001357.2 | 13514819 | ILMN_7196 | 130328 | 0.0452 | 2.60 | 2.18 | 3.06 |
| UBE2Z | NM_023079.2 | 20149671 | ILMN_17384 | 1030504 | 0.0451 | 1.38 | 1.29 | 1.46 |
| CIAS1 | NM_004895.3 | 34878692 | ILMN_11278 | 3190520 | 0.0449 | 1.49 | 1.39 | 1.60 |
| LOC645367 | XM_932672.1 | 89058763 | ILMN_34862 | 2680379 | 0.0446 | 1.25 | 1.19 | 1.31 |
| LOC652506 | XM_941975.1 | 89062938 | ILMN_39645 | 4920593 | 0.0445 | 2.16 | 1.89 | 2.41 |
| ITGAX | NM_000887.3 | 34452172 | ILMN_7741 | 270373 | 0.0443 | 1.44 | 1.35 | 1.54 |
| WBSCR20B | NM_145645.1 | 21717802 | ILMN_137520 | 6060452 | 0.0441 | 1.46 | 1.35 | 1.58 |
| LOC654135 | XM_945932.1 | 88999049 | ILMN_31315 | 620360 | 0.0439 | 1.51 | 1.40 | 1.62 |
| AGPAT2 | NM_006412.3 | 68835055 | ILMN_6967 | 6860039 | 0.0439 | 2.18 | 1.90 | 2.48 |
| LOC652184 | XM_941546.1 | 89062473 | ILMN_46021 | 7320553 | 0.0439 | 2.07 | 1.81 | 2.36 |
| TOP1 | NM_003286.2 | 19913404 | ILMN_13071 | 5890326 | 0.0438 | 1.97 | 1.74 | 2.21 |
| LOC645600 | XM_928616.1 | 89031346 | ILMN_31564 | 2750594 | 0.0437 | 1.22 | 1.17 | 1.27 |
| SPTBN1 | NM_178313.1 | 30315657 | ILMN_17508 | 4480091 | 0.0431 | 1.35 | 1.27 | 1.44 |
| GPR27 | NM_018971.1 | 9506746 | ILMN_16834 | 2600670 | 0.0428 | 2.14 | 1.88 | 2.44 |
| SMYD2 | NM_020197.1 | 9910273 | ILMN_4244 | 6900050 | 0.0428 | 1.61 | 1.45 | 1.76 |
| MAT2B | NM_182796.1 | 33519454 | ILMN_19777 | 4610133 | 0.0426 | 1.60 | 1.45 | 1.75 |
| LOC644615 | XM_927730.1 | 89035568 | ILMN_44684 | 4560414 | 0.042 | 1.41 | 1.32 | 1.49 |
| DNAJB12 | XM_944538.1 | 89031976 | ILMN_137399 | 3360204 | 0.0419 | 1.97 | 1.74 | 2.23 |
| LOC650230 | XM_941946.1 | 88970975 | ILMN_39890 | 5390315 | 0.0419 | 2.40 | 1.99 | 2.88 |
| PSMC4 | NM_153001.1 | 24430154 | ILMN_27399 | 6180192 | 0.0415 | 2.73 | 2.20 | 3.32 |
| USF2 | NM_003367.2 | 46877103 | ILMN_7790 | 5220079 | 0.0414 | 1.31 | 1.24 | 1.39 |
| PHF17 | NM_199320.1 | 40556392 | ILMN_26400 | 7200709 | 0.0412 | 1.54 | 1.42 | 1.66 |
| PIK3R5 | NM_014308.1 | 7657432 | ILMN_21503 | 6650564 | 0.0407 | 1.70 | 1.55 | 1.84 |
| LOC375133 | XM_942088.1 | 89071779 | ILMN_32434 | 3400632 | 0.0405 | 2.14 | 1.84 | 2.47 |
| C7ORF20 | NM_015949.2 | 38570061 | ILMN_23467 | 6660377 | 0.0405 | 1.44 | 1.34 | 1.53 |
| CASC4 | NM_138423.2 | 29826288 | ILMN_15514 | 3930458 | 0.0403 | 1.93 | 1.69 | 2.18 |
| CUGBP1 | NM_198700.1 | 38570080 | ILMN_10496 | 450243 | 0.0403 | 1.41 | 1.32 | 1.50 |
| HIATL2 | XM_939817.1 | 89030482 | ILMN_137017 | 1170750 | 0.0399 | 2.23 | 1.90 | 2.58 |
| CASP8 | NM_033358.2 | 73623022 | ILMN_29186 | 1300750 | 0.0397 | 3.46 | 2.66 | 4.37 |
| LIMK2 | NM_005569.3 | 73390104 | ILMN_5825 | 3840475 | 0.0396 | 1.35 | 1.27 | 1.43 |
| HCAP-H2 | NM_014551.3 | 34303963 | ILMN_14918 | 1850685 | 0.0394 | 1.28 | 1.21 | 1.35 |
| CASP8 | NM_033356.2 | 73623020 | ILMN_2110 | 2120719 | 0.0393 | 2.60 | 2.14 | 3.11 |
| NFATC2IP | NM_032815.3 | 46447822 | ILMN_17542 | 7160671 | 0.0393 | 1.34 | 1.27 | 1.40 |
| MAWBP | NM_001033083.1 | 74316008 | ILMN_12511 | 2120184 | 0.0391 | 1.19 | 1.14 | 1.24 |
| SIGIRR | NM_021805.1 | 11141876 | ILMN_18194 | 7380328 | 0.0391 | 2.60 | 2.20 | 3.05 |
| HS.569340 | DA483022 | 80904863 | ILMN_121521 | 4280181 | 0.039 | 1.21 | 1.16 | 1.26 |
| BTBD1 | NM_025238.3 | 59814019 | ILMN_19868 | 5860717 | 0.039 | 2.17 | 1.86 | 2.51 |
| ERBB2IP | NM_018695.2 | 56237019 | ILMN_26248 | 450646 | 0.0388 | 1.75 | 1.57 | 1.95 |
| AMY2B | NM_020978.3 | 56550100 | ILMN_5982 | 2970192 | 0.0386 | 1.45 | 1.35 | 1.55 |
| ATP1B3 | NM_001679.2 | 49574492 | ILMN_3785 | 5490403 | 0.0386 | 1.89 | 1.65 | 2.15 |
| AFF1 | NM_005935.1 | 5174572 | ILMN_8254 | 3130070 | 0.0385 | 1.25 | 1.19 | 1.31 |
| PML | XM_945882.1 | 89039091 | ILMN_137695 | 3400017 | 0.0384 | 1.24 | 1.18 | 1.30 |
| LOC643025 | XM_926168.1 | 89060501 | ILMN_38834 | 380243 | 0.0383 | 2.79 | 2.30 | 3.36 |
| UGP2 | NM_001001521.1 | 48255967 | ILMN_24547 | 7400035 | 0.0379 | 1.41 | 1.30 | 1.51 |
| STARD7 | NM_020151.2 | 21450854 | ILMN_9703 | 130707 | 0.0378 | 1.49 | 1.37 | 1.60 |
| SLC25A24 | NM_013386.2 | 33598953 | ILMN_15753 | 6400017 | 0.0377 | 1.19 | 1.14 | 1.23 |
| DMXL2 | NM_015263.1 | 19745147 | ILMN_24373 | 3060360 | 0.0376 | 2.29 | 1.98 | 2.63 |
| APOL6 | NM_030641.2 | 22035660 | ILMN_138012 | 6380338 | 0.0376 | 1.47 | 1.36 | 1.58 |
| AZIN1 | NM_015878.4 | 62526034 | ILMN_4825 | 5810504 | 0.0375 | 2.19 | 1.88 | 2.49 |
| PARP8 | NM_024615.2 | 24432008 | ILMN_26673 | 630671 | 0.0373 | 1.37 | 1.27 | 1.46 |
| LOC653504 | XM_930804.1 | 89059736 | ILMN_35114 | 6220255 | 0.0372 | 1.35 | 1.27 | 1.43 |
| KAT3 | NM_001008661.1 | 56713253 | ILMN_1120 | 6220474 | 0.0372 | 1.47 | 1.37 | 1.58 |
| POLDIP3 | NM_032311.3 | 30089917 | ILMN_11068 | 630743 | 0.0372 | 1.51 | 1.39 | 1.62 |
| IHPK2 | NM_001005911.1 | 55769523 | ILMN_4437 | 3130521 | 0.0369 | 2.12 | 1.83 | 2.45 |
| CXCR4 | NM_003467.2 | 56790928 | ILMN_26085 | 6650142 | 0.0369 | 4.26 | 3.19 | 5.69 |
| VHL | NM_000551.2 | 38045904 | ILMN_21046 | 5670746 | 0.0367 | 2.19 | 1.90 | 2.50 |
| TGIF | NM_003244.2 | 28178841 | ILMN_9308 | 4230014 | 0.0366 | 1.18 | 1.13 | 1.22 |
| DUSP6 | NM_001946.2 | 42764682 | ILMN_5440 | 4780754 | 0.0366 | 3.72 | 2.90 | 4.62 |
| AMD1 | NM_001634.4 | 74275345 | ILMN_21529 | 1430021 | 0.0365 | 3.43 | 2.63 | 4.42 |
| TSC1 | NM_001008567.1 | 56699467 | ILMN_24230 | 5080452 | 0.0365 | 1.20 | 1.16 | 1.24 |
| YWHAE | NM_006761.3 | 34304385 | ILMN_18524 | 160372 | 0.0364 | 1.37 | 1.29 | 1.45 |
| GPIAP1 | NM_203364.2 | 61676202 | ILMN_9771 | 5810438 | 0.0363 | 2.50 | 2.09 | 2.97 |
| SDHA | NM_004168.1 | 4759079 | ILMN_22058 | 1660341 | 0.0362 | 2.75 | 2.27 | 3.27 |
| HS.580138 | DA783170 | 82134687 | ILMN_132319 | 6580634 | 0.0362 | 1.47 | 1.37 | 1.58 |
| SLC25A3 | NM_213611.1 | 47132594 | ILMN_18748 | 1230196 | 0.0361 | 1.47 | 1.36 | 1.58 |
| MCL1 | NM_021960.3 | 33519459 | ILMN_18397 | 6020280 | 0.0361 | 5.20 | 3.84 | 6.81 |
| GALNACT-2 | NM_018590.3 | 24429591 | ILMN_11419 | 6060730 | 0.0356 | 2.37 | 2.00 | 2.75 |
| DNAJB12 | NM_017626.3 | 50593535 | ILMN_22702 | 2340750 | 0.0355 | 1.77 | 1.58 | 1.99 |
| SLC25A24 | NM_013386.2 | 33598953 | ILMN_15753 | 380376 | 0.0355 | 2.04 | 1.80 | 2.32 |
| LOC646358 | XM_929287.1 | 89038440 | ILMN_30990 | 1940520 | 0.0354 | 1.22 | 1.17 | 1.26 |
| LOC440836 | NM_001014440.1 | 62198217 | ILMN_26695 | 5960025 | 0.0354 | 1.25 | 1.19 | 1.30 |
| MGC5139 | XM_934229.1 | 89035770 | ILMN_39523 | 6040053 | 0.0352 | 1.12 | 1.09 | 1.15 |
| CTNNB1 | XM_945650.1 | 88968748 | ILMN_137682 | 6400066 | 0.0352 | 2.27 | 1.96 | 2.62 |
| TOP1MT | XM_944877.1 | 89028998 | ILMN_137050 | 60343 | 0.035 | 1.31 | 1.19 | 1.40 |
| BCL2L1 | NM_138578.1 | 20336334 | ILMN_12148 | 60162 | 0.0349 | 1.64 | 1.47 | 1.82 |
| PRIM2A | XM_942683.1 | 88999106 | ILMN_139106 | 3290273 | 0.0348 | 1.51 | 1.38 | 1.64 |
| DHX40 | NM_024612.3 | 31542728 | ILMN_1864 | 6280639 | 0.0348 | 1.66 | 1.49 | 1.82 |
| SLC25A3 | NM_213612.1 | 47132596 | ILMN_19383 | 7000167 | 0.0348 | 1.25 | 1.19 | 1.31 |
| RASSF5 | NM_182665.1 | 32996734 | ILMN_2837 | 7560215 | 0.0348 | 2.69 | 2.22 | 3.18 |
| RFFL | NM_001017368.1 | 62865648 | ILMN_18313 | 7000059 | 0.0347 | 1.17 | 1.12 | 1.22 |
| HIST2H2BF | NM_001024599.1 | 66912161 | ILMN_138755 | 3850021 | 0.0346 | 1.46 | 1.34 | 1.59 |
| SNX14 | NM_153816.2 | 39777616 | ILMN_590 | 4900040 | 0.0345 | 1.19 | 1.14 | 1.23 |
| KIAA0319L | NM_024874.3 | 33359220 | ILMN_21669 | 3370470 | 0.0344 | 2.39 | 2.04 | 2.77 |
| PIM3 | NM_001001852.2 | 52138581 | ILMN_19535 | 4250735 | 0.0344 | 1.68 | 1.52 | 1.85 |
| SLC39A9 | NM_018375.2 | 40254927 | ILMN_2302 | 6290369 | 0.0344 | 1.31 | 1.24 | 1.37 |
| IFRD1 | NM_001007245.1 | 55953130 | ILMN_21701 | 1820685 | 0.0342 | 1.90 | 1.67 | 2.14 |
| LOC651559 | XM_940732.1 | 89036328 | ILMN_37739 | 4610678 | 0.0342 | 3.69 | 2.88 | 4.65 |
| BACH1 | NM_001011545.1 | 59559716 | ILMN_19165 | 4920041 | 0.0342 | 1.66 | 1.48 | 1.84 |
| KIAA2010 | NM_017936.3 | 47933393 | ILMN_24293 | 870368 | 0.0341 | 1.66 | 1.51 | 1.82 |
| PARP6 | NM_020214.1 | 19482155 | ILMN_5230 | 3180632 | 0.034 | 1.25 | 1.19 | 1.30 |
| ARNTL | NM_001030273.1 | 71852581 | ILMN_6868 | 4480288 | 0.0338 | 1.25 | 1.19 | 1.30 |
| ZNF3 | NM_017715.1 | 8923203 | ILMN_21977 | 2640743 | 0.0337 | 1.44 | 1.33 | 1.55 |
| C20ORF32 | NM_020356.2 | 55769584 | ILMN_18849 | 730692 | 0.0336 | 1.58 | 1.44 | 1.73 |
| LOC649270 | XM_945399.1 | 89042789 | ILMN_31115 | 150475 | 0.0335 | 1.33 | 1.24 | 1.41 |
| IDH1 | NM_005896.2 | 28178824 | ILMN_14217 | 70605 | 0.0335 | 1.92 | 1.68 | 2.18 |
| NBR1 | NM_031862.1 | 14110380 | ILMN_16223 | 2570576 | 0.0334 | 1.50 | 1.39 | 1.62 |
| HRB | NM_004504.3 | 38570131 | ILMN_10703 | 6520685 | 0.0332 | 1.88 | 1.67 | 2.09 |
| TSN | NM_004622.2 | 20302160 | ILMN_14998 | 4780184 | 0.0329 | −1.17 | −1.22 | −1.13 |
| LOC648196 | XM_937246.1 | 89065640 | ILMN_36633 | 2680204 | 0.0328 | 1.99 | 1.78 | 2.23 |
| RASSF4 | NM_032023.3 | 30474868 | ILMN_2116 | 60239 | 0.0328 | 1.26 | 1.21 | 1.32 |
| HNRPK | NM_031263.1 | 14165436 | ILMN_16515 | 2970474 | 0.0326 | 2.51 | 2.08 | 2.96 |
| ZFYVE1 | NM_021260.1 | 30795179 | ILMN_6420 | 4540687 | 0.0325 | 1.35 | 1.27 | 1.44 |
| LOC391045 | XM_372780.3 | 88942847 | ILMN_35251 | 7650615 | 0.0325 | 2.24 | 1.91 | 2.60 |
| LOC653740 | XM_929347.1 | 88965790 | ILMN_30701 | 6180601 | 0.0324 | 1.17 | 1.13 | 1.21 |
| HK1 | NM_000188.1 | 4504390 | ILMN_26711 | 150379 | 0.0323 | 1.37 | 1.28 | 1.47 |
| SCP2 | NM_002979.3 | 56243511 | ILMN_1160 | 5690678 | 0.0323 | 1.56 | 1.40 | 1.73 |
| LOC653450 | XM_370557.2 | 89031217 | ILMN_43543 | 870392 | 0.0322 | 2.26 | 1.94 | 2.60 |
| AMFR | NM_001144.3 | 21071000 | ILMN_138270 | 380095 | 0.0321 | 1.43 | 1.32 | 1.53 |
| GRAP | XM_941681.1 | 89070432 | ILMN_137142 | 3870390 | 0.0321 | 1.59 | 1.43 | 1.77 |
| LOC654114 | XM_942070.1 | 88971006 | ILMN_40430 | 4860243 | 0.032 | 1.19 | 1.14 | 1.23 |
| FCGR2A | NM_021642.2 | 50511935 | ILMN_26366 | 2190035 | 0.0318 | 3.57 | 2.81 | 4.44 |
| LOC650274 | XM_942068.1 | 89034934 | ILMN_39625 | 6590762 | 0.0318 | 1.54 | 1.42 | 1.65 |
| C21ORF33 | NM_004649.4 | 38026968 | ILMN_28752 | 2120619 | 0.0317 | 1.89 | 1.62 | 2.21 |
| SPAG9 | NM_172345.1 | 27436921 | ILMN_21524 | 4050564 | 0.0317 | 2.02 | 1.78 | 2.28 |
| LOC652455 | XM_941904.1 | 89062811 | ILMN_32683 | 4060138 | 0.0317 | 1.41 | 1.31 | 1.52 |
| PMS1 | NM_000534.3 | 53729349 | ILMN_18417 | 6380424 | 0.0317 | −1.25 | −1.33 | −1.18 |
| LMAN1 | NM_005570.2 | 10862689 | ILMN_26805 | 2230703 | 0.0316 | 1.44 | 1.32 | 1.56 |
| LOC648154 | XM_943879.1 | 88952787 | ILMN_36960 | 4810184 | 0.0314 | 1.59 | 1.44 | 1.74 |
| ALG1 | NM_019109.3 | 41350215 | ILMN_20216 | 3610671 | 0.0313 | 1.49 | 1.38 | 1.61 |
| TLR8 | NM_016610.2 | 20302165 | ILMN_19246 | 580240 | 0.0313 | 1.51 | 1.36 | 1.65 |
| RNF6 | NM_183045.1 | 34305296 | ILMN_3182 | 2850692 | 0.0312 | 1.47 | 1.34 | 1.58 |
| SHC1 | NM_183001.3 | 52693920 | ILMN_8375 | 3360392 | 0.0312 | 1.41 | 1.32 | 1.51 |
| ACTR3B | NM_020445.3 | 54792124 | ILMN_21594 | 3400240 | 0.0312 | 1.31 | 1.23 | 1.39 |
| FAF1 | NM_007051.2 | 19528653 | ILMN_25532 | 3420372 | 0.0312 | 2.00 | 1.75 | 2.27 |
| CLK3 | NM_003992.1 | 4502884 | ILMN_1044 | 3990647 | 0.0311 | 2.71 | 2.30 | 3.17 |
| ZSWIM3 | XM_938235.1 | 89058075 | ILMN_137711 | 7330241 | 0.031 | 1.22 | 1.17 | 1.27 |
| GPR97 | XM_936582.1 | 89065470 | ILMN_138901 | 4640446 | 0.0307 | 1.15 | 1.11 | 1.19 |
| SC4MOL | NM_006745.3 | 62865626 | ILMN_2770 | 2000369 | 0.0305 | 1.54 | 1.40 | 1.68 |
| TBRG4 | NM_004749.2 | 40217811 | ILMN_27685 | 3850349 | 0.0305 | 1.28 | 1.22 | 1.35 |
| ARRDC2 | NM_015683.1 | 18373304 | ILMN_7560 | 6560685 | 0.0305 | 1.23 | 1.18 | 1.29 |
| MRFAP1L1 | NM_203462.1 | 44921607 | ILMN_9584 | 6130180 | 0.03 | 1.61 | 1.46 | 1.76 |
| MDFIC | NM_199072.2 | 40068513 | ILMN_21649 | 6250504 | 0.03 | 2.01 | 1.73 | 2.32 |
| LOC648024 | XM_943353.1 | 89060903 | ILMN_36327 | 4560196 | 0.0299 | 2.81 | 2.26 | 3.44 |
| SOD2 | NM_000636.2 | 67782304 | ILMN_19880 | 4640402 | 0.0299 | 3.92 | 3.01 | 4.93 |
| SPTLC1 | NM_178324.1 | 30474870 | ILMN_7889 | 3290397 | 0.0298 | 1.91 | 1.67 | 2.17 |
| LOC644422 | XM_930254.1 | 89041203 | ILMN_34341 | 4210497 | 0.0297 | 1.44 | 1.34 | 1.54 |
| SMPD1 | NM_000543.3 | 56117839 | ILMN_10742 | 5870168 | 0.0297 | 1.33 | 1.24 | 1.42 |
| IVNS1ABP | NM_016389.2 | 54144641 | ILMN_26908 | 2710242 | 0.0296 | 1.49 | 1.37 | 1.61 |
| GLE1L | NM_001499.2 | 51317381 | ILMN_18966 | 7510369 | 0.0296 | 1.67 | 1.50 | 1.86 |
| YWHAZ | NM_003406.2 | 21735623 | ILMN_11028 | 6660603 | 0.0293 | 2.83 | 2.36 | 3.36 |
| VNN2 | NM_004665.2 | 17865813 | ILMN_16565 | 1400070 | 0.0292 | 5.99 | 4.25 | 8.30 |
| NGRN | NM_016645.2 | 49574506 | ILMN_14282 | 3460102 | 0.0291 | 2.07 | 1.76 | 2.40 |
| SRP9 | NM_003133.1 | 4507216 | ILMN_137290 | 4610358 | 0.029 | 1.89 | 1.64 | 2.15 |
| LOC653150 | XM_931089.1 | 88998561 | ILMN_46899 | 1850446 | 0.0285 | 1.16 | 1.12 | 1.20 |
| (WTAP) | | | | | | | | |
| CD82 | NM_002231.3 | 67782352 | ILMN_24985 | 3310427 | 0.0285 | 1.87 | 1.65 | 2.09 |
| SNX6 | NM_021249.2 | 23111048 | ILMN_139240 | 2060039 | 0.0284 | 1.38 | 1.29 | 1.47 |
| PPP2R5C | NM_002719.2 | 31083258 | ILMN_18075 | 4010348 | 0.0284 | 2.12 | 1.80 | 2.47 |
| GPR15 | NM_005290.1 | 4885298 | ILMN_10602 | 6980291 | 0.0282 | 1.61 | 1.43 | 1.79 |
| HPCAL1 | NM_002149.2 | 19913440 | ILMN_11657 | 1820148 | 0.028 | 1.66 | 1.51 | 1.82 |
| TSPAN4 | NM_001025234.1 | 68799996 | ILMN_9896 | 7200544 | 0.0279 | 1.24 | 1.19 | 1.30 |
| RASSF1 | NM_170713.1 | 25777681 | ILMN_14262 | 2480619 | 0.0278 | 1.68 | 1.51 | 1.84 |
| WRNIP1 | NM_020135.2 | 18426901 | ILMN_30297 | 6180605 | 0.0278 | 1.89 | 1.67 | 2.15 |
| PARVB | NM_013327.3 | 51477694 | ILMN_545 | 2470634 | 0.0274 | 1.16 | 1.12 | 1.20 |
| CNN2 | NM_004368.2 | 41327728 | ILMN_26898 | 870241 | 0.0272 | 3.53 | 2.80 | 4.38 |
| PPIL2 | NM_148175.1 | 22547211 | ILMN_4210 | 50609 | 0.0271 | 1.13 | 1.10 | 1.17 |
| TAF4B | XM_290809.5 | 89047154 | ILMN_39525 | 7040471 | 0.0269 | −1.16 | −1.20 | −1.12 |
| C10ORF26 | NM_017787.3 | 41152103 | ILMN_28562 | 7150671 | 0.0269 | 2.32 | 1.94 | 2.75 |
| FEN1 | NM_004111.4 | 19718776 | ILMN_24738 | 1820521 | 0.0268 | 1.34 | 1.25 | 1.44 |
| LOC654347 | XM_946379.1 | 89034108 | ILMN_44485 | 2350446 | 0.0267 | 1.45 | 1.35 | 1.56 |
| GALK2 | NM_002044.2 | 48527955 | ILMN_10957 | 4900129 | 0.0267 | 1.62 | 1.46 | 1.78 |
| SVH | NM_031905.2 | 31377662 | ILMN_16544 | 6770079 | 0.0267 | 1.42 | 1.31 | 1.52 |
| CDK5RAP1 | NM_016408.2 | 28872781 | ILMN_7499 | 7210128 | 0.0267 | 1.22 | 1.16 | 1.27 |
| YEARSDAILY- | NA | NA | YEARSDAILY- | YEARSDAILY- | 0.0267 | 16.40 | 12.20 | 20.60 |
| SMOKING | | | SMOKING | SMOKING | | | | |
| FKBP1A | NM_054014.1 | 17149835 | ILMN_29213 | 110475 | 0.0266 | 2.52 | 2.05 | 3.08 |
| ACSL3 | NM_004457.3 | 42794751 | ILMN_416 | 540112 | 0.0266 | 1.31 | 1.23 | 1.39 |
| FBXO38 | NM_205836.1 | 45545408 | ILMN_4585 | 3800187 | 0.0265 | 1.46 | 1.36 | 1.57 |
| CR1 | XM_936516.1 | 88952714 | ILMN_137312 | 610687 | 0.0265 | 2.49 | 2.10 | 2.93 |
| MAP2K3 | NM_145109.1 | 21618348 | ILMN_14315 | 5390561 | 0.0264 | 1.49 | 1.38 | 1.60 |
| RPSA | NM_001012321.1 | 59859884 | ILMN_20469 | 70307 | 0.0264 | −1.22 | −1.28 | −1.16 |
| TRPM7 | NM_017672.2 | 29893551 | ILMN_11670 | 610187 | 0.0262 | 1.28 | 1.21 | 1.35 |
| CASP9 | NM_001229.2 | 14790123 | ILMN_2760 | 770754 | 0.0262 | 1.52 | 1.41 | 1.63 |
| SLIC1 | NM_182854.1 | 33504570 | ILMN_511 | 1980338 | 0.026 | 2.21 | 1.87 | 2.58 |
| BTN2A2 | NM_006995.3 | 31881700 | ILMN_15223 | 4220494 | 0.026 | 1.29 | 1.23 | 1.36 |
| ENTPD6 | NM_001247.1 | 4557422 | ILMN_17684 | 6480669 | 0.026 | 1.20 | 1.15 | 1.25 |
| CR1 | NM_000651.3 | 21536275 | ILMN_137353 | 770075 | 0.026 | 1.61 | 1.46 | 1.78 |
| ZNF655 | NM_001009956.1 | 58331255 | ILMN_2214 | 1780370 | 0.0259 | 1.13 | 1.09 | 1.17 |
| APOL2 | NM_145637.1 | 22035652 | ILMN_19232 | 5870376 | 0.0259 | 1.63 | 1.49 | 1.77 |
| CHMP6 | NM_024591.3 | 52851447 | ILMN_26654 | 510142 | 0.0256 | 1.57 | 1.44 | 1.71 |
| SERTAD3 | NM_203344.1 | 42741651 | ILMN_1527 | 1190634 | 0.0254 | 1.78 | 1.59 | 1.97 |
| IFIT3 | NM_001031683.1 | 72534657 | ILMN_22925 | 3830041 | 0.0254 | 2.69 | 2.16 | 3.31 |
| GFM2 | NM_170681.1 | 25306282 | ILMN_16025 | 4070735 | 0.0254 | 1.41 | 1.30 | 1.52 |
| TAGAP | NM_138810.2 | 23199968 | ILMN_11224 | 4250369 | 0.0254 | 3.30 | 2.58 | 4.13 |
| UBE2L6 | NM_004223.3 | 38157980 | ILMN_7531 | 20110 | 0.0252 | 2.91 | 2.35 | 3.56 |
| BCL6 | NM_138931.1 | 21040335 | ILMN_18289 | 4640044 | 0.025 | 1.23 | 1.16 | 1.30 |
| AP1S1 | NM_001283.2 | 16950626 | ILMN_21653 | 6270301 | 0.025 | 1.51 | 1.38 | 1.63 |
| NOD27 | NM_032206.2 | 28951070 | ILMN_23914 | 6650445 | 0.025 | 2.13 | 1.83 | 2.41 |
| STX16 | NM_001001433.1 | 47778942 | ILMN_12925 | 3290307 | 0.0249 | 1.39 | 1.30 | 1.48 |
| HIATL2 | NM_032318.1 | 14150087 | ILMN_138936 | 6480390 | 0.0249 | 1.93 | 1.69 | 2.19 |
| C1ORF58 | NM_144695.1 | 21389600 | ILMN_11942 | 2710612 | 0.0248 | 1.52 | 1.37 | 1.67 |
| OASL | NM_003733.2 | 38016933 | ILMN_4735 | 7150196 | 0.0248 | 1.85 | 1.65 | 2.07 |
| LOC255809 | XM_930239.1 | 89052292 | ILMN_31703 | 4900114 | 0.0247 | 2.16 | 1.86 | 2.51 |
| PTPN22 | NM_012411.2 | 15619017 | ILMN_25877 | 6100338 | 0.0247 | 1.99 | 1.72 | 2.27 |
| LOC440349 | XM_496129.2 | 89040448 | ILMN_40146 | 6380239 | 0.0246 | 1.33 | 1.24 | 1.42 |
| PDE7A | NM_002603.1 | 24429565 | ILMN_13515 | 2350646 | 0.0245 | 1.64 | 1.49 | 1.81 |
| LOC554223 | XR_001115.1 | 88998673 | ILMN_42290 | 1510341 | 0.0244 | 3.62 | 2.82 | 4.60 |
| RAB27A | NM_183235.1 | 34485708 | ILMN_13878 | 1580730 | 0.0244 | 1.37 | 1.28 | 1.46 |
| NPEPPS | NM_006310.2 | 15451906 | ILMN_8237 | 2190519 | 0.0244 | 1.31 | 1.24 | 1.37 |
| SLC39A3 | NM_144564.4 | 47080101 | ILMN_27676 | 3520605 | 0.0243 | 1.66 | 1.51 | 1.83 |
| IL1RN | NM_173842.1 | 27894318 | ILMN_3867 | 2190653 | 0.0241 | 3.72 | 2.92 | 4.71 |
| THAP4 | NM_015963.4 | 47059038 | ILMN_8784 | 540452 | 0.0241 | 1.23 | 1.17 | 1.29 |
| BMX | NM_203281.1 | 42544181 | ILMN_11912 | 1110341 | 0.024 | 1.15 | 1.11 | 1.20 |
| LOC652615 | XM_942150.1 | 89072185 | ILMN_40224 | 7160039 | 0.024 | 1.43 | 1.32 | 1.53 |
| ARHGAP25 | NM_014882.2 | 55770896 | ILMN_14823 | 7570280 | 0.024 | 1.35 | 1.28 | 1.42 |
| TSC22D3 | NM_001015881.1 | 62865624 | ILMN_20126 | 3800707 | 0.0239 | 1.40 | 1.29 | 1.52 |
| LBH | NM_030915.1 | 13569871 | ILMN_21350 | 4120086 | 0.0239 | 1.58 | 1.43 | 1.74 |
| SNAP23 | NM_003825.2 | 18765728 | ILMN_29211 | 4490053 | 0.0237 | 3.78 | 2.87 | 4.99 |
| DNTTIP2 | NM_014597.3 | 54633314 | ILMN_26105 | 2260411 | 0.0236 | −1.17 | −1.22 | −1.13 |
| MLL3 | NM_170606.1 | 24586652 | ILMN_14020 | 6330332 | 0.0236 | 2.20 | 1.91 | 2.52 |
| MAGED2 | NM_177433.1 | 29171704 | ILMN_16101 | 2630132 | 0.0235 | 1.29 | 1.22 | 1.36 |
| PPP2R2D | NM_018461.2 | 51093850 | ILMN_22358 | 3460026 | 0.0234 | 2.06 | 1.77 | 2.37 |
| TRIM5 | NM_033092.1 | 15011943 | ILMN_29177 | 4040095 | 0.0233 | 1.25 | 1.19 | 1.32 |
| LIMK2 | NM_001031801.1 | 73390139 | ILMN_6284 | 5390349 | 0.0233 | 2.41 | 2.02 | 2.82 |
| ATF2 | NM_001880.2 | 22538421 | ILMN_12901 | 1450546 | 0.0232 | 1.67 | 1.49 | 1.87 |
| ATP2A2 | NM_001681.2 | 27886536 | ILMN_4412 | 4250093 | 0.0232 | 1.67 | 1.49 | 1.87 |
| PPP1R9B | NM_032595.1 | 14211926 | ILMN_138367 | 5820717 | 0.0231 | 1.25 | 1.18 | 1.32 |
| MEF2A | NM_005587.1 | 5031906 | ILMN_17271 | 1450291 | 0.023 | 1.38 | 1.29 | 1.47 |
| HP1BP3 | NM_016287.2 | 56676329 | ILMN_29502 | 150291 | 0.023 | 1.85 | 1.62 | 2.09 |
| CRLF3 | NM_015986.2 | 27764872 | ILMN_22668 | 4390397 | 0.023 | 2.89 | 2.37 | 3.53 |
| C9ORF77 | NM_016014.2 | 71051599 | ILMN_12685 | 4850008 | 0.023 | 1.63 | 1.47 | 1.78 |
| ADAM17 | NM_003183.4 | 73747888 | ILMN_5977 | 2900468 | 0.0229 | 1.30 | 1.23 | 1.38 |
| METRNL | XM_941466.1 | 89043124 | ILMN_42199 | 1170288 | 0.0228 | 1.97 | 1.72 | 2.27 |
| HIATL2 | NM_032318.1 | 14150087 | ILMN_138936 | 3460424 | 0.0228 | 2.24 | 1.89 | 2.59 |
| DHRS9 | NM_199204.1 | 40548396 | ILMN_25196 | 1300746 | 0.0227 | 1.69 | 1.50 | 1.90 |
| SP3 | NM_003111.3 | 67078401 | ILMN_15345 | 2060768 | 0.0227 | 1.41 | 1.31 | 1.51 |
| FYN | NM_153047.1 | 23510361 | ILMN_25662 | 1090372 | 0.0226 | 2.75 | 2.21 | 3.36 |
| CDC42EP3 | NM_006449.3 | 30089964 | ILMN_1066 | 1780072 | 0.0226 | 2.02 | 1.75 | 2.32 |
| HS.559151 | AW292488 | 6699124 | ILMN_113570 | 5130402 | 0.0224 | 1.53 | 1.40 | 1.68 |
| CASP6 | NM_032992.2 | 73622127 | ILMN_11438 | 5860113 | 0.0224 | −1.34 | −1.43 | −1.25 |
| VPS16 | NM_022575.2 | 17978478 | ILMN_11344 | 4210524 | 0.0223 | 1.62 | 1.47 | 1.77 |
| LOC653650 | XM_935348.1 | 89039623 | ILMN_45794 | 2320066 | 0.0222 | 1.98 | 1.73 | 2.27 |
| PRKCD | NM_006254.3 | 47157323 | ILMN_17715 | 1770554 | 0.0221 | 1.22 | 1.17 | 1.27 |
| Sep. 7, 2010 | NM_001011553.1 | 58535460 | ILMN_24703 | 2680754 | 0.0221 | −1.74 | −1.99 | −1.49 |
| FLJ38973 | NM_153689.3 | 31581540 | ILMN_23846 | 7160577 | 0.0221 | −1.17 | −1.22 | −1.13 |
| USP21 | NM_001014443.2 | 74027268 | ILMN_18137 | 7330504 | 0.0221 | 1.17 | 1.13 | 1.21 |
| MANEA | NM_024641.2 | 41393555 | ILMN_6991 | 620474 | 0.022 | 1.46 | 1.35 | 1.57 |
| LOC648022 | XM_943614.1 | 88952757 | ILMN_36041 | 1030243 | 0.0219 | 1.84 | 1.64 | 2.03 |
| LOC644614 | XM_927729.1 | 88943047 | ILMN_42864 | 4670373 | 0.0219 | 1.12 | 1.09 | 1.16 |
| CD200R1 | NM_138940.2 | 68215643 | ILMN_3165 | 1570687 | 0.0218 | 1.47 | 1.35 | 1.59 |
| GMPR2 | NM_016576.3 | 50541955 | ILMN_1280 | 870551 | 0.0218 | 1.15 | 1.11 | 1.18 |
| LOC642323 | XM_925863.1 | 88943701 | ILMN_32302 | 6590066 | 0.0217 | 1.50 | 1.36 | 1.64 |
| NFS1 | NM_181679.1 | 32307129 | ILMN_3492 | 6980053 | 0.0217 | 1.27 | 1.19 | 1.34 |
| LOC650654 | XM_939739.1 | 89039101 | ILMN_37996 | 5490121 | 0.0216 | −1.22 | −1.28 | −1.16 |
| VNN3 | NM_078625.2 | 66932886 | ILMN_25942 | 2060600 | 0.0215 | 1.55 | 1.41 | 1.69 |
| CXORF40B | NM_001013845.1 | 62241037 | ILMN_12545 | 4640068 | 0.0215 | 1.20 | 1.14 | 1.26 |
| LOC653942 | XM_938116.1 | 89033520 | ILMN_39856 | 360382 | 0.0214 | 2.67 | 2.15 | 3.31 |
| CCR4 | NM_005508.4 | 48762930 | ILMN_10745 | 6270246 | 0.0214 | 1.33 | 1.22 | 1.44 |
| LOC643025 | XM_926168.1 | 89060501 | ILMN_38834 | 7550139 | 0.0214 | 2.39 | 2.00 | 2.82 |
| ROCK1 | NM_005406.1 | 4885582 | ILMN_23091 | 6250497 | 0.0213 | 1.77 | 1.58 | 1.98 |
| REPS2 | NM_004726.1 | 4758943 | ILMN_21036 | 6450220 | 0.0213 | 2.01 | 1.74 | 2.33 |
| MCTP2 | NM_018349.2 | 50657351 | ILMN_3204 | 2570338 | 0.0212 | 1.80 | 1.60 | 2.02 |
| XKR8 | NM_018053.2 | 24431976 | ILMN_11071 | 2350338 | 0.0211 | 1.40 | 1.30 | 1.50 |
| LOC645625 | XM_935208.1 | 89041729 | ILMN_35948 | 130070 | 0.021 | 1.65 | 1.48 | 1.81 |
| RAB37 | NM_175738.3 | 54859694 | ILMN_520 | 4570279 | 0.021 | 1.21 | 1.16 | 1.26 |
| FABP5 | NM_001444.1 | 4557580 | ILMN_27564 | 2350040 | 0.0209 | −1.13 | −1.16 | −1.09 |
| MCCC2 | NM_022132.3 | 14251210 | ILMN_19445 | 290400 | 0.0209 | 1.32 | 1.23 | 1.40 |
| UBXD7 | XM_931517.1 | 88967344 | ILMN_36533 | 4490564 | 0.0207 | 1.36 | 1.27 | 1.44 |
| OPN3 | NM_014322.2 | 71999130 | ILMN_26561 | 5220170 | 0.0205 | 1.44 | 1.33 | 1.56 |
| TMLHE | NM_018196.1 | 8922624 | ILMN_29460 | 840053 | 0.0199 | 1.28 | 1.21 | 1.36 |
| LOC553158 | NM_181334.3 | 66346696 | ILMN_2156 | 2750253 | 0.0198 | 1.36 | 1.27 | 1.45 |
| LSP1 | NM_001013253.1 | 61742788 | ILMN_12132 | 5720192 | 0.0198 | 2.51 | 2.04 | 3.03 |
| MICAL2 | NM_014632.2 | 41281417 | ILMN_27460 | 4010753 | 0.0197 | 1.50 | 1.38 | 1.63 |
| DERPC | NM_017804.3 | 50811884 | ILMN_25110 | 4290673 | 0.0197 | 1.65 | 1.49 | 1.82 |
| UBL7 | NM_032907.3 | 41152105 | ILMN_17890 | 4900348 | 0.0197 | 1.76 | 1.57 | 1.95 |
| GTDC1 | NM_001006636.1 | 54859762 | ILMN_13831 | 6660154 | 0.0197 | 1.26 | 1.19 | 1.33 |
| HS.570636 | AK023371 | 10435278 | ILMN_122817 | 2750068 | 0.0194 | 1.35 | 1.26 | 1.44 |
| ATRX | NM_138270.1 | 20336204 | ILMN_16109 | 6020156 | 0.0194 | 1.39 | 1.29 | 1.49 |
| PPM1G | NM_177983.1 | 29826281 | ILMN_878 | 1300470 | 0.0193 | 1.45 | 1.34 | 1.57 |
| DPP7 | XM_939309.1 | 89030620 | ILMN_137782 | 1440102 | 0.0193 | 1.90 | 1.64 | 2.20 |
| IFIT3 | NM_001549.2 | 31542979 | ILMN_1944 | 430021 | 0.0193 | 4.44 | 3.29 | 5.85 |
| HERC3 | NM_014606.1 | 7657151 | ILMN_21657 | 4860138 | 0.0192 | 1.88 | 1.67 | 2.13 |
| CCNDBP1 | NM_037370.1 | 16554567 | ILMN_23609 | 4760520 | 0.0191 | 1.20 | 1.15 | 1.26 |
| UNC45A | NM_017979.1 | 8922201 | ILMN_27819 | 670255 | 0.0191 | 1.32 | 1.24 | 1.38 |
| HNRPA3 | NM_194247.1 | 34740328 | ILMN_5256 | 1070138 | 0.019 | 1.89 | 1.65 | 2.16 |
| C15ORF44 | XM_940546.1 | 89039133 | ILMN_138325 | 1340477 | 0.019 | 1.18 | 1.14 | 1.23 |
| PPP2R5D | NM_006245.2 | 31083266 | ILMN_5210 | 1410411 | 0.019 | 1.53 | 1.39 | 1.67 |
| CUGBP2 | NM_001025076.1 | 68303644 | ILMN_21178 | 5700392 | 0.0189 | 1.19 | 1.13 | 1.25 |
| PPM1A | NM_177952.1 | 29557938 | ILMN_10552 | 580520 | 0.0189 | 1.47 | 1.36 | 1.58 |
| TGFBR2 | NM_001024847.1 | 67782325 | ILMN_22189 | 7100403 | 0.0189 | 1.49 | 1.35 | 1.63 |
| TRADD | NM_003789.2 | 24234723 | ILMN_27933 | 3610020 | 0.0188 | 2.39 | 2.01 | 2.80 |
| GRIPAP1 | NM_020137.3 | 46592990 | ILMN_27811 | 4730215 | 0.0188 | 1.53 | 1.42 | 1.63 |
| MATK | NM_139355.1 | 21450845 | ILMN_13609 | 7650424 | 0.0188 | 1.82 | 1.62 | 2.05 |
| TBL2 | NM_032988.1 | 14670378 | ILMN_136934 | 5090703 | 0.0187 | 1.52 | 1.39 | 1.65 |
| PHC2 | NM_004427.2 | 37595529 | ILMN_28897 | 6650739 | 0.0185 | 1.55 | 1.42 | 1.68 |
| RPLP1 | NM_001003.2 | 16905511 | ILMN_23181 | 6560114 | 0.0184 | −2.29 | −2.78 | −1.86 |
| PPP1R3B | NM_024607.1 | 13375814 | ILMN_9571 | 1070626 | 0.0182 | 1.54 | 1.40 | 1.70 |
| AP1GBP1 | NM_007247.3 | 38569408 | ILMN_13930 | 2350474 | 0.0182 | 1.68 | 1.51 | 1.87 |
| LOC651621 | XM_940809.1 | 89031867 | ILMN_45641 | 3840215 | 0.0182 | 1.43 | 1.31 | 1.54 |
| TSC22D1 | NM_006022.2 | 31543826 | ILMN_26720 | 4200719 | 0.0182 | 2.58 | 2.12 | 3.17 |
| CUTL1 | NM_001913.2 | 31652235 | ILMN_8630 | 4850189 | 0.018 | 1.39 | 1.29 | 1.48 |
| LOC647100 | XM_930115.1 | 89040267 | ILMN_34067 | 4900577 | 0.0179 | −2.26 | −2.73 | −1.82 |
| RPL27A | NM_000990.2 | 14141189 | ILMN_139166 | 3420367 | 0.0178 | −2.18 | −2.60 | −1.82 |
| DUSP10 | NM_007207.3 | 21536334 | ILMN_17179 | 5420242 | 0.0178 | 1.22 | 1.16 | 1.28 |
| BIRC2 | NM_001166.3 | 41349435 | ILMN_23760 | 2570064 | 0.0177 | 2.07 | 1.79 | 2.38 |
| MGC3123 | NM_177441.1 | 28973798 | ILMN_9166 | 3520386 | 0.0177 | 2.05 | 1.82 | 2.28 |
| PCK2 | NM_001018073.1 | 66346722 | ILMN_18787 | 60671 | 0.0177 | 1.33 | 1.25 | 1.42 |
| PSEN1 | NM_007319.1 | 7549814 | ILMN_762 | 5690561 | 0.0176 | 2.14 | 1.84 | 2.45 |
| LAIR1 | NM_021708.1 | 11231178 | ILMN_26463 | 2340646 | 0.0175 | 1.70 | 1.52 | 1.90 |
| RGS3 | NM_021106.3 | 62865652 | ILMN_2596 | 2000500 | 0.0173 | 1.14 | 1.10 | 1.17 |
| APOBEC3F | NM_001006666.1 | 54873618 | ILMN_18531 | 4070132 | 0.0172 | 1.46 | 1.31 | 1.62 |
| AGPAT3 | NM_020132.3 | 41327762 | ILMN_138486 | 2470113 | 0.0171 | 1.56 | 1.40 | 1.74 |
| LOC653542 | XM_927999.1 | 89058191 | ILMN_39385 | 4290364 | 0.0168 | 2.85 | 2.26 | 3.50 |
| SLCO3A1 | NM_013272.2 | 7706713 | ILMN_27392 | 870224 | 0.0168 | 2.01 | 1.73 | 2.31 |
| VRK3 | NM_016440.3 | 71164885 | ILMN_13129 | 3290487 | 0.0167 | 2.16 | 1.83 | 2.50 |
| DNASE1L1 | NM_006730.2 | 58430940 | ILMN_6814 | 3440341 | 0.0167 | 1.48 | 1.35 | 1.62 |
| ZNF274 | NM_133502.1 | 19743800 | ILMN_18462 | 1660184 | 0.0166 | 1.60 | 1.45 | 1.75 |
| LOC388344 | XM_371023.4 | 89041208 | ILMN_34544 | 2350719 | 0.0166 | −2.05 | −2.40 | −1.72 |
| CASP4 | NM_033306.2 | 73622124 | ILMN_7434 | 3610048 | 0.0166 | 1.70 | 1.50 | 1.93 |
| DALRD3 | NM_018114.4 | 58331231 | ILMN_12427 | 5390703 | 0.0166 | 1.48 | 1.37 | 1.60 |
| LOC641750 | XM_935596.1 | 89027461 | ILMN_31678 | 5720184 | 0.0166 | −1.70 | −1.93 | −1.48 |
| OGFOD1 | NM_001031707.1 | 72534703 | ILMN_16561 | 1570181 | 0.0163 | 1.14 | 1.10 | 1.19 |
| EHMT1 | NM_024757.3 | 40217807 | ILMN_18594 | 6420730 | 0.0163 | 1.25 | 1.19 | 1.31 |
| PHKB | NM_001031835.1 | 73611905 | ILMN_18544 | 1770703 | 0.0162 | 1.73 | 1.56 | 1.90 |
| RAB43 | NM_198490.1 | 50234888 | ILMN_12157 | 3290220 | 0.0161 | 1.59 | 1.45 | 1.74 |
| C1ORF80 | NM_022831.1 | 12383075 | ILMN_6651 | 4810246 | 0.0161 | 1.49 | 1.36 | 1.62 |
| LOC643300 | XM_931981.1 | 88982356 | ILMN_40497 | 770369 | 0.0159 | 1.31 | 1.23 | 1.39 |
| C1ORF108 | XM_941120.1 | 88948823 | ILMN_138824 | 2760128 | 0.0158 | 2.89 | 2.33 | 3.52 |
| ARMC8 | NM_014154.2 | 47458044 | ILMN_20531 | 130437 | 0.0157 | 1.97 | 1.71 | 2.26 |
| FLJ12886 | NM_019108.1 | 10092658 | ILMN_5022 | 2600279 | 0.0157 | 1.43 | 1.32 | 1.55 |
| CD8A | NM_001768.4 | 27886640 | ILMN_2358 | 540731 | 0.0157 | 2.99 | 2.37 | 3.70 |
| REEP3 | NM_001001330.1 | 47679088 | ILMN_4633 | 1850482 | 0.0156 | 1.39 | 1.28 | 1.51 |
| EPB41 | NM_203343.1 | 42716288 | ILMN_15301 | 240255 | 0.0156 | 1.99 | 1.72 | 2.27 |
| TAPBP | NM_172209.1 | 27436896 | ILMN_26682 | 7040731 | 0.0156 | 1.94 | 1.69 | 2.19 |
| LOC643668 | XM_928629.1 | 89035385 | ILMN_38401 | 2190181 | 0.0155 | 2.26 | 1.89 | 2.68 |
| RAB24 | NM_130781.1 | 18640747 | ILMN_21668 | 6350201 | 0.0154 | 1.96 | 1.67 | 2.28 |
| ANXA2 | NM_001002857.1 | 50845385 | ILMN_8830 | 5820403 | 0.0153 | 2.59 | 2.10 | 3.18 |
| F8A3 | NM_001007524.1 | 56090585 | ILMN_4937 | 7150161 | 0.0153 | 1.51 | 1.37 | 1.64 |
| LOC124491 | NM_145254.1 | 21687071 | ILMN_14043 | 2140484 | 0.0152 | 1.94 | 1.68 | 2.21 |
| TBC1D3 | NM_032258.1 | 14149984 | ILMN_26578 | 2470215 | 0.0151 | 1.46 | 1.33 | 1.58 |
| LOC650967 | XM_946056.1 | 89057446 | ILMN_31342 | 5900209 | 0.0151 | 1.53 | 1.38 | 1.67 |
| PLEKHB2 | NM_001031706.1 | 72534701 | ILMN_8325 | 6100240 | 0.0151 | 2.54 | 2.09 | 3.07 |
| HS.168950 | BC036496.1 | 71051952 | ILMN_80128 | 7560195 | 0.0151 | −1.13 | −1.16 | −1.09 |
| (GDAP1) | | | | | | | | |
| C3ORF28 | NM_014367.3 | 49355720 | ILMN_24382 | 5050047 | 0.015 | −1.47 | −1.63 | −1.33 |
| IL12RB1 | NM_005535.1 | 5031784 | ILMN_4594 | 5670647 | 0.015 | 1.79 | 1.55 | 2.06 |
| TRIM23 | NM_001656.3 | 44955890 | ILMN_24727 | 6280161 | 0.015 | 1.91 | 1.65 | 2.21 |
| PARG | NM_003631.2 | 70610135 | ILMN_8739 | 4010603 | 0.0149 | 1.24 | 1.18 | 1.31 |
| COMT | NM_000754.2 | 6466451 | ILMN_2463 | 2710603 | 0.0148 | 1.68 | 1.51 | 1.85 |
| SSFA2 | NM_006751.3 | 34222128 | ILMN_4525 | 5700343 | 0.0148 | 1.79 | 1.59 | 1.98 |
| TNFRSF10C | NM_003841.2 | 22547120 | ILMN_4106 | 520553 | 0.0147 | 1.64 | 1.46 | 1.82 |
| GALK2 | NM_001001556.1 | 48527956 | ILMN_6492 | 5700039 | 0.0147 | 1.31 | 1.23 | 1.38 |
| INPP4A | NM_001566.1 | 4504704 | ILMN_19517 | 1740048 | 0.0145 | 1.31 | 1.23 | 1.39 |
| LOC51136 | NM_016125.2 | 21361528 | ILMN_27239 | 2750403 | 0.0145 | 2.09 | 1.77 | 2.45 |
| PLD3 | NM_001031696.1 | 72534683 | ILMN_6460 | 4780037 | 0.0144 | 1.26 | 1.20 | 1.32 |
| DIABLO | NM_138930.2 | 42544194 | ILMN_19433 | 2480041 | 0.0142 | 1.66 | 1.47 | 1.86 |
| LOC651575 | XM_940750.1 | 89066735 | ILMN_33487 | 4210041 | 0.0142 | 1.23 | 1.15 | 1.30 |
| LOC124216 | XR_001518.1 | 89039499 | ILMN_37388 | 6270768 | 0.0142 | 1.73 | 1.56 | 1.92 |
| SFI1 | NM_014775.2 | 55956783 | ILMN_23938 | 630181 | 0.0142 | −1.32 | −1.42 | −1.22 |
| C1ORF9 | NM_014283.2 | 29837653 | ILMN_9240 | 6520424 | 0.0142 | −1.16 | −1.21 | −1.12 |
| SCAMP1 | NM_004866.3 | 33598919 | ILMN_28654 | 2690709 | 0.0141 | 1.45 | 1.33 | 1.60 |
| ARPC4 | NM_005718.3 | 68161505 | ILMN_24602 | 1070196 | 0.014 | 1.16 | 1.12 | 1.20 |
| FAM73A | NM_198549.1 | 38348383 | ILMN_4385 | 3940747 | 0.014 | −1.22 | −1.28 | −1.15 |
| PKNOX1 | NM_004571.3 | 37595549 | ILMN_29895 | 6250379 | 0.014 | 1.21 | 1.16 | 1.26 |
| SERPINA1 | NM_001002236.1 | 50363218 | ILMN_30268 | 2060592 | 0.0139 | 2.50 | 2.04 | 3.01 |
| FCGR2A | XM_938849.1 | 88952546 | ILMN_138445 | 2100100 | 0.0139 | 3.82 | 2.92 | 5.00 |
| FBXL17 | NM_022824.1 | 45238579 | ILMN_8400 | 610164 | 0.0139 | 1.15 | 1.10 | 1.19 |
| PIK3C2A | NM_002645.1 | 4505798 | ILMN_2470 | 6270181 | 0.0139 | 1.45 | 1.33 | 1.56 |
| LOC650020 | XM_939111.1 | 88952884 | ILMN_40049 | 2480152 | 0.0138 | 1.43 | 1.32 | 1.55 |
| LOC642998 | XM_931228.1 | 88995794 | ILMN_38499 | 2140170 | 0.0137 | 1.13 | 1.10 | 1.17 |
| SULT1A1 | NM_177536.1 | 29540542 | ILMN_29763 | 5270477 | 0.0137 | 1.26 | 1.20 | 1.32 |
| C17ORF60 | XM_945975.1 | 89042847 | ILMN_33002 | 130010 | 0.0136 | 1.91 | 1.60 | 2.26 |
| WDR43 | XM_944889.1 | 88954702 | ILMN_43073 | 4210164 | 0.0136 | 1.42 | 1.31 | 1.54 |
| LOC652826 | XM_942509.1 | 89064749 | ILMN_34282 | 4250373 | 0.0136 | 2.14 | 1.83 | 2.49 |
| SFRS11 | NM_004768.2 | 23111060 | ILMN_4847 | 1400626 | 0.0134 | −1.45 | −1.59 | −1.31 |
| COG5 | NM_006348.2 | 32481215 | ILMN_10374 | 4920463 | 0.0134 | 1.42 | 1.31 | 1.55 |
| CASP8 | NM_001228.3 | 73623018 | ILMN_29639 | 650241 | 0.0134 | 1.36 | 1.27 | 1.44 |
| GOLGA7 | NM_016099.2 | 50541949 | ILMN_30279 | 1170619 | 0.0133 | 1.36 | 1.28 | 1.45 |
| HSPBP1 | NM_012267.2 | 21361406 | ILMN_19625 | 160543 | 0.0133 | 1.21 | 1.15 | 1.27 |
| MOCS2 | NM_176806.2 | 35493763 | ILMN_27055 | 3450484 | 0.0133 | 1.53 | 1.39 | 1.68 |
| RAB33B | NM_031296.1 | 13786128 | ILMN_21878 | 3930138 | 0.0133 | −1.60 | −1.85 | −1.38 |
| CLSTN1 | NM_001009566.1 | 57242756 | ILMN_29098 | 6760600 | 0.0133 | 1.25 | 1.17 | 1.31 |
| TALDO1 | XM_938697.1 | 89034447 | ILMN_138767 | 1010491 | 0.0132 | 1.59 | 1.41 | 1.77 |
| JAK1 | NM_002227.1 | 4504802 | ILMN_554 | 2510246 | 0.0132 | 3.31 | 2.58 | 4.17 |
| LOC652613 | XM_942146.1 | 89063256 | ILMN_42004 | 4890576 | 0.0131 | 2.07 | 1.81 | 2.36 |
| SPN | NM_003123.3 | 71892475 | ILMN_19780 | 7210192 | 0.0131 | 1.22 | 1.16 | 1.29 |
| FAM18B | NM_016078.3 | 71061433 | ILMN_18985 | 1190706 | 0.013 | 1.91 | 1.63 | 2.22 |
| DOK2 | NM_003974.2 | 41406049 | ILMN_21820 | 3890605 | 0.013 | 1.63 | 1.47 | 1.81 |
| LOC647392 | XM_942791.1 | 88987475 | ILMN_41904 | 6270685 | 0.0129 | 1.16 | 1.12 | 1.20 |
| CGI-09 | NM_015939.3 | 29244922 | ILMN_29842 | 1440100 | 0.0128 | −1.16 | −1.22 | −1.10 |
| LOC651319 | XM_944594.1 | 88957160 | ILMN_45901 | 2190424 | 0.0128 | 1.24 | 1.17 | 1.30 |
| CTDSP1 | NM_182642.1 | 32813442 | ILMN_13739 | 3610072 | 0.0128 | 1.83 | 1.61 | 2.06 |
| RPS6KA3 | XM_944112.1 | 89060584 | ILMN_137549 | 7330136 | 0.0128 | 2.37 | 2.02 | 2.78 |
| LOC388122 | XM_370865.3 | 89038392 | ILMN_46143 | 5290064 | 0.0127 | −1.16 | −1.20 | −1.11 |
| HNRPUL1 | NM_144732.1 | 21536319 | ILMN_4191 | 6180091 | 0.0127 | 1.46 | 1.31 | 1.62 |
| ATP1A1 | NM_000701.6 | 48762680 | ILMN_677 | 6370121 | 0.0127 | 1.95 | 1.67 | 2.27 |
| HDLBP | NM_005336.2 | 42716278 | ILMN_5820 | 6960411 | 0.0127 | 1.19 | 1.14 | 1.25 |
| IL1R2 | NM_004633.3 | 27894332 | ILMN_25995 | 5960754 | 0.0126 | 1.95 | 1.66 | 2.27 |
| LOC648081 | XM_937132.1 | 88951496 | ILMN_33238 | 7560598 | 0.0126 | 1.89 | 1.66 | 2.14 |
| OGT | NM_003605.3 | 32307145 | ILMN_4667 | 1050168 | 0.0124 | 1.13 | 1.09 | 1.17 |
| CD151 | NM_004357.3 | 34328913 | ILMN_139293 | 4830504 | 0.0124 | 1.98 | 1.72 | 2.26 |
| ADK | NM_001123.2 | 32484972 | ILMN_4107 | 6840209 | 0.0124 | 1.34 | 1.24 | 1.45 |
| DBR1 | NM_016216.2 | 56549112 | ILMN_19003 | 6130494 | 0.0122 | 1.63 | 1.46 | 1.82 |
| BLR1 | NM_001716.2 | 14589867 | ILMN_27589 | 1440291 | 0.0121 | 1.34 | 1.21 | 1.47 |
| EZH2 | NM_004456.3 | 23510382 | ILMN_25740 | 580296 | 0.0121 | 1.23 | 1.17 | 1.29 |
| LOC653276 | XM_931495.1 | 89035740 | ILMN_34785 | 2570717 | 0.012 | 1.38 | 1.28 | 1.49 |
| GLT8D1 | NM_001010983.1 | 58331224 | ILMN_8696 | 3930754 | 0.012 | 1.19 | 1.14 | 1.24 |
| RQCD1 | NM_005444.1 | 4885578 | ILMN_29301 | 3990243 | 0.0119 | 1.36 | 1.27 | 1.45 |
| RAB39B | NM_171998.2 | 64762487 | ILMN_22924 | 2690142 | 0.0117 | −1.15 | −1.19 | −1.11 |
| NR3C1 | NM_001018076.1 | 66528585 | ILMN_6719 | 6550079 | 0.0117 | 1.22 | 1.17 | 1.27 |
| HS.574855 | DN917404 | 77945616 | ILMN_127036 | 4070192 | 0.0115 | 1.11 | 1.07 | 1.15 |
| NEDD9 | NM_182966.1 | 33667052 | ILMN_137978 | 4570091 | 0.0115 | 1.24 | 1.18 | 1.30 |
| ILF3 | NM_153464.1 | 24234755 | ILMN_24364 | 4810139 | 0.0115 | 1.41 | 1.31 | 1.51 |
| LOC196264 | NM_198275.1 | 38093644 | ILMN_23862 | 2340131 | 0.0114 | 1.89 | 1.66 | 2.15 |
| AP1S1 | NM_001283.2 | 16950626 | ILMN_21653 | 2650075 | 0.0114 | 1.48 | 1.36 | 1.61 |
| LOC653382 | XM_934354.1 | 89042081 | ILMN_44569 | 5220243 | 0.0114 | 1.26 | 1.19 | 1.33 |
| SMARCD3 | NM_003078.3 | 51477705 | ILMN_8015 | 1400605 | 0.0113 | 1.58 | 1.40 | 1.78 |
| ILF3 | NM_004516.2 | 24234752 | ILMN_12252 | 5090168 | 0.0113 | 1.87 | 1.61 | 2.15 |
| SNX15 | NM_013306.3 | 46370087 | ILMN_1967 | 2750605 | 0.0112 | 1.20 | 1.15 | 1.26 |
| LOC652773 | XM_942415.1 | 89077406 | ILMN_38539 | 7610167 | 0.0112 | 1.49 | 1.36 | 1.63 |
| LOC651023 | XM_940136.1 | 89030314 | ILMN_40631 | 2120048 | 0.0111 | 1.23 | 1.17 | 1.30 |
| CDKAL1 | NM_017774.1 | 8923317 | ILMN_26274 | 4120445 | 0.0111 | 1.56 | 1.41 | 1.72 |
| CDKN2D | NM_001800.3 | 39995074 | ILMN_28866 | 1500364 | 0.011 | 1.91 | 1.69 | 2.14 |
| SORD | NM_003104.3 | 34147623 | ILMN_27787 | 4260075 | 0.011 | 1.38 | 1.28 | 1.49 |
| HS.514843 | BX094382 | 27841938 | ILMN_98745 | 2680400 | 0.0109 | 1.50 | 1.37 | 1.65 |
| CEP57 | NM_014679.3 | 59710114 | ILMN_27141 | 6370445 | 0.0109 | −1.34 | −1.45 | −1.24 |
| KIAA0564 | NM_015058.1 | 57863270 | ILMN_16560 | 7380594 | 0.0109 | −1.27 | −1.36 | −1.18 |
| HNRPA1 | NM_031157.1 | 14043069 | ILMN_138150 | 610400 | 0.0108 | 1.38 | 1.26 | 1.50 |
| ARF4 | NM_001660.2 | 6995998 | ILMN_5548 | 2490243 | 0.0107 | 1.96 | 1.66 | 2.32 |
| LOC649095 | XM_945154.1 | 89059185 | ILMN_32679 | 580709 | 0.0107 | 1.64 | 1.45 | 1.87 |
| SUMO2 | NM_001005849.1 | 54792070 | ILMN_16713 | 1070181 | 0.0106 | −2.02 | −2.39 | −1.65 |
| LOC653743 | XM_929369.1 | 88953184 | ILMN_34703 | 1770068 | 0.0106 | 1.79 | 1.54 | 2.04 |
| MCM7 | NM_005916.3 | 33469967 | ILMN_1986 | 2360278 | 0.0106 | 1.23 | 1.16 | 1.30 |
| HS.562444 | AI961125 | 5753763 | ILMN_115550 | 4120300 | 0.0106 | 1.28 | 1.21 | 1.36 |
| LOC388621 | XM_371243.4 | 88942623 | ILMN_43918 | 4180564 | 0.0106 | −2.05 | −2.46 | −1.66 |
| CCT7 | NM_006429.2 | 58331183 | ILMN_22959 | 7150017 | 0.0106 | 2.81 | 2.26 | 3.45 |
| SF3B1 | NM_001005526.1 | 54112118 | ILMN_13059 | 7150072 | 0.0106 | 1.96 | 1.68 | 2.25 |
| SIAH1 | NM_003031.3 | 63148617 | ILMN_18192 | 7200398 | 0.0106 | 1.18 | 1.14 | 1.22 |
| CPT1A | NM_001876.2 | 73623029 | ILMN_14446 | 6130450 | 0.0105 | 1.14 | 1.10 | 1.18 |
| RBMS1 | NM_002897.3 | 46249390 | ILMN_18726 | 1500411 | 0.0104 | 1.28 | 1.18 | 1.37 |
| UTP11L | NM_016037.2 | 52856412 | ILMN_2243 | 2190554 | 0.0104 | −1.25 | −1.32 | −1.18 |
| ING3 | NM_198267.1 | 38201658 | ILMN_23155 | 5820113 | 0.0104 | 1.83 | 1.62 | 2.06 |
| STAM2 | NM_005843.3 | 21265030 | ILMN_9193 | 4480608 | 0.0103 | 1.65 | 1.48 | 1.85 |
| PTPRA | NM_002836.2 | 18450367 | ILMN_8330 | 6060603 | 0.0103 | 1.36 | 1.27 | 1.44 |
| C3ORF23 | NM_001029839.1 | 71067097 | ILMN_10936 | 1170128 | 0.0102 | 1.54 | 1.39 | 1.68 |
| SERPINA1 | NM_000295.3 | 50363216 | ILMN_1034 | 1470719 | 0.0102 | 1.23 | 1.16 | 1.30 |
| OPA3 | NM_025136.1 | 13376716 | ILMN_11296 | 4150189 | 0.0102 | 1.11 | 1.07 | 1.15 |
| ERCC8 | NM_001007234.1 | 55956772 | ILMN_5204 | 4120292 | 0.0101 | 1.25 | 1.18 | 1.33 |
| HMBS | NM_000190.3 | 66933007 | ILMN_16358 | 4560315 | 0.0101 | 1.47 | 1.35 | 1.60 |
| LOC649707 | XM_938775.1 | 89059247 | ILMN_34741 | 1050142 | 0.01 | 1.16 | 1.12 | 1.20 |
| LOC644295 | XM_927468.1 | 89037300 | ILMN_38707 | 380390 | 0.01 | 1.22 | 1.15 | 1.28 |
| CCT6A | NM_001762.3 | 58331169 | ILMN_21650 | 70347 | 0.01 | 2.22 | 1.85 | 2.63 |
| MLKL | XM_936963.1 | 89041041 | ILMN_139138 | 780148 | 0.01 | 1.25 | 1.19 | 1.32 |
| HS.570385 | DA674107 | 80937528 | ILMN_122566 | 6450408 | 0.0099 | 2.18 | 1.87 | 2.53 |
| HS.385555 | BC035378 | 23273407 | ILMN_89057 | 2710148 | 0.0098 | 1.30 | 1.21 | 1.39 |
| LOC646144 | XM_935294.1 | 89025359 | ILMN_45775 | 2750152 | 0.0098 | 1.14 | 1.09 | 1.19 |
| ZBTB41 | NM_194314.2 | 61743929 | ILMN_7261 | 2100471 | 0.0097 | −1.21 | −1.28 | −1.15 |
| DAP3 | NM_033657.1 | 16905525 | ILMN_13395 | 270528 | 0.0097 | 1.65 | 1.48 | 1.84 |
| LOC644037 | XM_933604.1 | 88983852 | ILMN_37144 | 5390719 | 0.0097 | 2.91 | 2.29 | 3.71 |
| LOC648294 | XM_939952.1 | 89030185 | ILMN_36674 | 6330133 | 0.0097 | −2.14 | −2.61 | −1.73 |
| SIGLEC7 | NM_014385.1 | 7657569 | ILMN_29432 | 5860538 | 0.0096 | 1.29 | 1.21 | 1.37 |
| PDLIM2 | NM_021630.4 | 40288188 | ILMN_11298 | 3930564 | 0.0095 | 1.15 | 1.11 | 1.20 |
| DNAJC11 | NM_018198.1 | 8922628 | ILMN_14957 | 3290136 | 0.0093 | 1.26 | 1.19 | 1.32 |
| LIG4 | NM_002312.3 | 46255050 | ILMN_25322 | 5670129 | 0.0093 | 1.22 | 1.17 | 1.28 |
| SFRS12 | NM_139168.1 | 21040254 | ILMN_8967 | 6420356 | 0.0093 | −1.24 | −1.35 | −1.15 |
| TMEM23 | NM_147156.3 | 41350331 | ILMN_26608 | 4540102 | 0.0092 | 1.26 | 1.19 | 1.33 |
| RIOK1 | NM_031480.2 | 23510355 | ILMN_8030 | 4780593 | 0.0092 | 1.27 | 1.19 | 1.36 |
| QKI | XM_942223.1 | 88999422 | ILMN_45956 | 1660746 | 0.0091 | 1.72 | 1.52 | 1.94 |
| KIAA1432 | NM_020829.1 | 75832028 | ILMN_26728 | 1820026 | 0.0091 | 1.12 | 1.08 | 1.16 |
| CSNK1A1 | NM_001892.4 | 68303571 | ILMN_24977 | 4850092 | 0.0091 | 1.99 | 1.68 | 2.36 |
| BRD4 | NM_014299.1 | 7657217 | ILMN_19745 | 5360523 | 0.0091 | 1.20 | 1.15 | 1.25 |
| KLHL7 | NM_001031710.1 | 72534709 | ILMN_8698 | 2760411 | 0.009 | −1.32 | −1.41 | −1.22 |
| CCM2 | NM_031443.3 | 71067339 | ILMN_4086 | 4040681 | 0.009 | 1.63 | 1.47 | 1.80 |
| CES1 | NM_001025194.1 | 68508964 | ILMN_4194 | 4670402 | 0.009 | 1.35 | 1.25 | 1.46 |
| TRPV4 | NM_147204.1 | 22547179 | ILMN_649 | 7320291 | 0.0089 | 1.35 | 1.25 | 1.45 |
| IHPK1 | NM_153273.3 | 58530860 | ILMN_1661 | 2120433 | 0.0088 | 1.33 | 1.25 | 1.42 |
| APP | NM_000484.2 | 41406053 | ILMN_30235 | 7210167 | 0.0087 | 1.23 | 1.16 | 1.28 |
| C4ORF13 | NM_001030316.1 | 71896704 | ILMN_11185 | 4120025 | 0.0086 | 1.18 | 1.13 | 1.23 |
| PPP1CA | NM_002708.3 | 45827796 | ILMN_26836 | 5570035 | 0.0086 | 2.26 | 1.88 | 2.73 |
| LOC643035 | XM_931996.1 | 88943744 | ILMN_33896 | 2100022 | 0.0085 | −1.69 | −1.90 | −1.47 |
| LOC642684 | XM_926137.1 | 89025519 | ILMN_34902 | 5290661 | 0.0085 | 1.83 | 1.61 | 2.08 |
| QKI | NM_206854.1 | 45827709 | ILMN_4669 | 6660097 | 0.0085 | 1.91 | 1.67 | 2.16 |
| HS.570444 | AJ003554 | 2792050 | ILMN_122625 | 670477 | 0.0084 | 1.23 | 1.15 | 1.32 |
| PTMA | NM_002823.2 | 21359859 | ILMN_7102 | 730129 | 0.0084 | −2.42 | −3.00 | −1.90 |
| LOC651726 | XM_940945.1 | 89062121 | ILMN_42644 | 1090050 | 0.0083 | 1.39 | 1.29 | 1.49 |
| RPL23 | NM_000978.2 | 14591907 | ILMN_137528 | 4120707 | 0.0083 | −1.92 | −2.29 | −1.58 |
| LOC651816 | XM_941060.1 | 89062188 | ILMN_46354 | 6110053 | 0.0083 | −1.16 | −1.22 | −1.11 |
| CASP10 | NM_032974.2 | 47078268 | ILMN_13756 | 6770253 | 0.0083 | 1.20 | 1.15 | 1.25 |
| LOC642112 | XM_936252.1 | 89026476 | ILMN_33587 | 4220148 | 0.0082 | 1.91 | 1.68 | 2.18 |
| TIA1 | NM_022173.1 | 11863162 | ILMN_29910 | 1030358 | 0.0081 | 1.84 | 1.58 | 2.12 |
| RBM3 | NM_001017430.1 | 63054839 | ILMN_15994 | 2970356 | 0.0081 | 1.47 | 1.35 | 1.60 |
| CCS | NM_005125.1 | 4826664 | ILMN_23509 | 4200286 | 0.0081 | 1.84 | 1.58 | 2.13 |
| LOC650155 | XM_939236.1 | 89032028 | ILMN_35338 | 4610753 | 0.0081 | 1.47 | 1.35 | 1.59 |
| C14ORF124 | NM_020195.1 | 9910257 | ILMN_4144 | 6590270 | 0.0081 | 1.40 | 1.28 | 1.53 |
| CRSP8 | XM_933599.1 | 88983845 | ILMN_45168 | 7400739 | 0.0081 | 1.64 | 1.45 | 1.84 |
| NCF1 | NM_000265.1 | 4557784 | ILMN_136961 | 1230538 | 0.008 | 3.66 | 2.71 | 4.88 |
| LOC652537 | XM_942027.1 | 88971364 | ILMN_31258 | 3290600 | 0.0079 | 1.16 | 1.11 | 1.20 |
| CLEC7A | NM_197953.1 | 37675384 | ILMN_3417 | 1090170 | 0.0078 | 1.95 | 1.59 | 2.41 |
| RNU108 | NR_002324.1 | 68342028 | ILMN_19266 | 160326 | 0.0078 | 1.20 | 1.15 | 1.26 |
| CPEB4 | NM_030627.1 | 32698754 | ILMN_5007 | 1690360 | 0.0078 | 2.01 | 1.72 | 2.35 |
| HPCAL1 | NM_134421.1 | 19913442 | ILMN_12582 | 240019 | 0.0078 | 1.23 | 1.15 | 1.30 |
| CSNK2A1 | NM_177559.2 | 47419901 | ILMN_30267 | 2750767 | 0.0078 | 1.15 | 1.10 | 1.20 |
| BCR | NM_004327.2 | 11038638 | ILMN_136932 | 4250463 | 0.0078 | 1.16 | 1.12 | 1.21 |
| LOC641949 | XM_935713.1 | 89026832 | ILMN_45778 | 6660162 | 0.0078 | 1.24 | 1.19 | 1.30 |
| C6ORF106 | NM_024294.2 | 46094084 | ILMN_24069 | 6770070 | 0.0078 | 1.18 | 1.13 | 1.23 |
| LOC642817 | XM_926703.1 | 88990450 | ILMN_46700 | 1190079 | 0.0077 | 2.82 | 2.17 | 3.64 |
| ALDH3B1 | NM_000694.2 | 71773289 | ILMN_27131 | 1780202 | 0.0077 | 1.20 | 1.14 | 1.26 |
| SNX11 | NM_152244.1 | 23111027 | ILMN_9237 | 5690280 | 0.0077 | 1.21 | 1.16 | 1.27 |
| LOC653328 | XM_926913.1 | 88942611 | ILMN_43519 | 7320709 | 0.0077 | −1.51 | −1.70 | −1.34 |
| NNT | NM_012343.2 | 33695083 | ILMN_20204 | 10674 | 0.0076 | −1.12 | −1.16 | −1.09 |
| CXORF53 | NM_024332.2 | 64762482 | ILMN_18443 | 1820541 | 0.0076 | 1.14 | 1.10 | 1.18 |
| IQWD1 | NM_018442.2 | 63252907 | ILMN_16460 | 5490068 | 0.0076 | 1.20 | 1.13 | 1.26 |
| TMCC1 | NM_001017395.1 | 62859976 | ILMN_29162 | 6290131 | 0.0076 | 1.36 | 1.26 | 1.47 |
| HS.550193 | U43604 | 1171236 | ILMN_110215 | 1450088 | 0.0075 | 1.34 | 1.22 | 1.48 |
| LOC641913 | XM_935667.1 | 89026774 | ILMN_43603 | 2810605 | 0.0075 | 1.17 | 1.12 | 1.23 |
| FLJ11712 | NM_024570.1 | 13375741 | ILMN_20578 | 4010600 | 0.0075 | −1.61 | −1.83 | −1.40 |
| LOC647743 | XM_936805.1 | 89065527 | ILMN_46476 | 510753 | 0.0075 | 1.56 | 1.40 | 1.72 |
| LOC644482 | XM_927612.1 | 88943848 | ILMN_37198 | 5560097 | 0.0075 | −1.17 | −1.22 | −1.12 |
| ZMAT1 | NM_032441.1 | 58533171 | ILMN_9028 | 6280603 | 0.0075 | −1.17 | −1.22 | −1.12 |
| LOC650696 | XM_944334.1 | 89031821 | ILMN_44076 | 940241 | 0.0075 | 1.31 | 1.21 | 1.40 |
| RNU64 | NR_002326.1 | 68510027 | ILMN_19397 | 10736 | 0.0074 | 1.30 | 1.21 | 1.39 |
| PLB1 | NM_153021.3 | 76096365 | ILMN_27755 | 3120600 | 0.0074 | 1.34 | 1.24 | 1.43 |
| C3ORF17 | NM_015412.3 | 75812961 | ILMN_26202 | 3420044 | 0.0074 | 1.19 | 1.15 | 1.25 |
| ABR | NM_001092.3 | 38679953 | ILMN_23502 | 3780131 | 0.0074 | 1.30 | 1.22 | 1.38 |
| TOP1MT | NM_052963.1 | 16418460 | ILMN_15321 | 4480465 | 0.0074 | 1.41 | 1.26 | 1.56 |
| SNCB | NM_001001502.1 | 48255902 | ILMN_8144 | 5960309 | 0.0074 | 1.30 | 1.21 | 1.40 |
| SBDSP | NR_001588.1 | 38348442 | ILMN_12233 | 1450102 | 0.0073 | 1.96 | 1.67 | 2.25 |
| HS.543405 | AA668142 | 2629641 | ILMN_107001 | 2350500 | 0.0073 | 1.04 | 1.01 | 1.06 |
| C17ORF80 | NM_017941.3 | 34222156 | ILMN_21070 | 4290609 | 0.0073 | 1.58 | 1.43 | 1.72 |
| SLC25A30 | NM_001010875.1 | 58197561 | ILMN_27751 | 610717 | 0.0073 | 1.14 | 1.10 | 1.19 |
| ADAM18 | NM_014237.1 | 7656860 | ILMN_5673 | 7150338 | 0.0073 | 1.28 | 1.19 | 1.36 |
| MTMR3 | NM_021090.2 | 23510385 | ILMN_27578 | 7330435 | 0.0073 | 1.49 | 1.36 | 1.62 |
| RAB27A | NM_183234.1 | 34485705 | ILMN_10265 | 1580619 | 0.0072 | 1.15 | 1.11 | 1.21 |
| AMACR | NM_203382.1 | 42822892 | ILMN_2954 | 2260039 | 0.0072 | 1.22 | 1.15 | 1.29 |
| LOC653141 | XM_926169.1 | 89040568 | ILMN_44352 | 4850112 | 0.0072 | 1.77 | 1.58 | 1.99 |
| FBXO7 | NM_001033024.1 | 74229028 | ILMN_28646 | 4920435 | 0.0072 | 1.36 | 1.23 | 1.49 |
| ITGB1 | NM_133376.1 | 19743822 | ILMN_11529 | 5890707 | 0.0072 | 2.94 | 2.29 | 3.70 |
| TFEC | NM_012252.2 | 64762384 | ILMN_15030 | 1240082 | 0.0071 | 1.11 | 1.09 | 1.14 |
| ZNF655 | NM_001009957.1 | 58331259 | ILMN_3621 | 1940138 | 0.0071 | 1.34 | 1.24 | 1.45 |
| LOC652481 | XM_941942.1 | 89062863 | ILMN_35551 | 3120056 | 0.0071 | 1.69 | 1.49 | 1.91 |
| ASB3 | NM_016115.3 | 22208952 | ILMN_25973 | 4640020 | 0.0071 | 1.49 | 1.36 | 1.63 |
| HNRPAB | NM_031266.2 | 55956918 | ILMN_757 | 540437 | 0.0071 | 1.31 | 1.19 | 1.43 |
| CPT1B | NM_152247.1 | 23238257 | ILMN_13033 | 2850468 | 0.007 | 1.25 | 1.18 | 1.32 |
| RIF1 | NM_018151.3 | 56676334 | ILMN_4664 | 3460307 | 0.007 | 1.19 | 1.14 | 1.24 |
| RB1 | NM_000321.1 | 4506434 | ILMN_4636 | 4260113 | 0.007 | 1.86 | 1.62 | 2.13 |
| LOC653117 | XM_931656.1 | 88986976 | ILMN_37789 | 4570487 | 0.007 | 2.21 | 1.80 | 2.68 |
| MAPKAP1 | NM_001006618.1 | 56788400 | ILMN_13996 | 50053 | 0.007 | 1.55 | 1.42 | 1.69 |
| CCL7 | NM_006273.2 | 13435401 | ILMN_24123 | 6590500 | 0.007 | 1.28 | 1.20 | 1.38 |
| PTPN6 | NM_080548.2 | 34328901 | ILMN_25213 | 6900291 | 0.007 | 1.29 | 1.21 | 1.38 |
| ZA20D3 | NM_019006.2 | 21359917 | ILMN_16822 | 7380577 | 0.007 | 2.25 | 1.82 | 2.74 |
| NUP50 | NM_153645.1 | 24497446 | ILMN_138009 | 2370463 | 0.0069 | 1.18 | 1.12 | 1.24 |
| CD74 | NM_001025159.1 | 68448543 | ILMN_21963 | 3420154 | 0.0069 | 2.05 | 1.72 | 2.45 |
| HS.579654 | AW887586 | 8049599 | ILMN_131835 | 160025 | 0.0068 | 1.31 | 1.21 | 1.42 |
| C15ORF23 | NM_033286.1 | 57528365 | ILMN_28790 | 6770176 | 0.0068 | 1.26 | 1.17 | 1.37 |
| B3GALT2 | NM_003783.2 | 15451871 | ILMN_14361 | 1010187 | 0.0067 | −1.20 | −1.27 | −1.14 |
| PHC2 | NM_198040.1 | 37595527 | ILMN_7686 | 3180735 | 0.0067 | 1.94 | 1.67 | 2.25 |
| EEF1B2 | NM_021121.2 | 16519563 | ILMN_138368 | 5690162 | 0.0067 | 1.54 | 1.38 | 1.73 |
| FAM19A2 | NM_178539.3 | 52486623 | ILMN_2438 | 5900731 | 0.0067 | 1.28 | 1.18 | 1.37 |
| C5ORF4 | NM_032385.1 | 14150216 | ILMN_16262 | 1710747 | 0.0066 | 1.22 | 1.16 | 1.27 |
| ASPSCR1 | NM_024083.2 | 17572803 | ILMN_9446 | 1820014 | 0.0066 | 1.15 | 1.10 | 1.20 |
| WBSCR16 | NM_030798.2 | 22538491 | ILMN_26391 | 5570408 | 0.0066 | 1.21 | 1.16 | 1.26 |
| LOC649986 | XM_939071.1 | 89066123 | ILMN_31648 | 2260082 | 0.0065 | 2.71 | 2.18 | 3.35 |
| MCM7 | NM_182776.1 | 33469921 | ILMN_1133 | 1690475 | 0.0064 | 1.15 | 1.09 | 1.20 |
| HS.560098 | BQ214365 | 20395765 | ILMN_114052 | 2630730 | 0.0064 | 1.25 | 1.19 | 1.32 |
| SH3BP2 | NM_003023.2 | 19923154 | ILMN_1151 | 6250201 | 0.0063 | 1.30 | 1.22 | 1.39 |
| WNT1 | NM_005430.2 | 16936523 | ILMN_22389 | 6380215 | 0.0063 | −1.08 | −1.12 | −1.05 |
| CDC2L1 | NM_033493.1 | 16332371 | ILMN_4002 | 1260041 | 0.0062 | 2.15 | 1.81 | 2.52 |
| ZNF278 | NM_032051.1 | 14670363 | ILMN_2933 | 2970520 | 0.0062 | 1.15 | 1.10 | 1.20 |
| ETFB | NM_001985.2 | 62420878 | ILMN_7194 | 1340044 | 0.0061 | 1.90 | 1.63 | 2.23 |
| KIAA1967 | NM_199205.1 | 40548407 | ILMN_15274 | 2690477 | 0.0061 | 1.10 | 1.05 | 1.14 |
| NCF4 | NM_013416.2 | 47519769 | ILMN_7892 | 3610102 | 0.0061 | 1.89 | 1.64 | 2.17 |
| LIPE | NM_005357.2 | 21328445 | ILMN_896 | 70047 | 0.0061 | −1.09 | −1.12 | −1.06 |
| CSDE1 | NM_001007553.1 | 56117851 | ILMN_3664 | 4780347 | 0.006 | 1.44 | 1.32 | 1.57 |
| ESM1 | NM_007036.2 | 13259505 | ILMN_138415 | 1090743 | 0.0059 | −1.13 | −1.17 | −1.09 |
| TRIM5 | NM_033034.1 | 14719417 | ILMN_760 | 2360598 | 0.0059 | 2.23 | 1.87 | 2.65 |
| HS.571222 | AB032973 | 71891696 | ILMN_123403 | 4640544 | 0.0059 | 1.15 | 1.11 | 1.20 |
| SOS2 | NM_006939.1 | 39930603 | ILMN_12037 | 7160114 | 0.0059 | 1.81 | 1.62 | 2.03 |
| RTN1 | NM_021136.2 | 45827774 | ILMN_2601 | 7210520 | 0.0059 | 1.20 | 1.15 | 1.26 |
| NOMO3 | NM_001004067.1 | 51944968 | ILMN_5042 | 4490035 | 0.0058 | 1.67 | 1.47 | 1.86 |
| SERPINB2 | NM_002575.1 | 4505594 | ILMN_14466 | 5090327 | 0.0058 | 1.39 | 1.26 | 1.52 |
| FBXW7 | NM_033632.2 | 61743923 | ILMN_7221 | 5270152 | 0.0058 | 1.42 | 1.29 | 1.56 |
| GPR109B | NM_006018.1 | 5174460 | ILMN_22584 | 5960360 | 0.0058 | 2.35 | 1.90 | 2.90 |
| LOC652253 | XM_941661.1 | 88955119 | ILMN_34827 | 6220220 | 0.0058 | −1.11 | −1.16 | −1.07 |
| NFKBIZ | NM_031419.2 | 53832022 | ILMN_18526 | 6380039 | 0.0058 | 1.19 | 1.13 | 1.25 |
| FLT3LG | NM_001459.2 | 38455415 | ILMN_4754 | 780544 | 0.0058 | −1.39 | −1.53 | −1.25 |
| MAP4K3 | NM_003618.2 | 15451901 | ILMN_5588 | 870095 | 0.0058 | 1.23 | 1.16 | 1.30 |
| TNPO1 | NM_002270.2 | 23510378 | ILMN_18758 | 460368 | 0.0057 | 1.11 | 1.07 | 1.15 |
| BIRC1 | XM_936944.1 | 88987995 | ILMN_137577 | 60541 | 0.0057 | 1.95 | 1.64 | 2.28 |
| C9ORF77 | NM_001025780.1 | 71051601 | ILMN_12321 | 870070 | 0.0057 | −1.46 | −1.63 | −1.30 |
| PMS2CL | XR_001272.1 | 89025732 | ILMN_39709 | 3520521 | 0.0056 | 1.22 | 1.17 | 1.28 |
| HS.445121 | BM545878 | 18778358 | ILMN_92941 | 4570136 | 0.0056 | 1.26 | 1.19 | 1.33 |
| EPIM | NM_194356.1 | 37577161 | ILMN_17438 | 4590241 | 0.0056 | 1.21 | 1.15 | 1.27 |
| GPR89A | NM_016334.2 | 56181388 | ILMN_10695 | 4780709 | 0.0056 | 1.48 | 1.33 | 1.63 |
| DDX17 | NM_030881.2 | 38201711 | ILMN_28024 | 7400475 | 0.0056 | 1.82 | 1.60 | 2.04 |
| C19ORF12 | NM_001031726.1 | 72534737 | ILMN_10211 | 1470605 | 0.0055 | 1.23 | 1.17 | 1.30 |
| SBDS | NM_016038.2 | 28416939 | ILMN_15766 | 20181 | 0.0055 | −1.57 | −1.77 | −1.36 |
| AKAP10 | NM_007202.2 | 21493032 | ILMN_5307 | 2120349 | 0.0055 | 1.82 | 1.59 | 2.09 |
| HS.170828 | AI498339 | 4390321 | ILMN_80247 | 5810706 | 0.0055 | −1.04 | −1.07 | −1.01 |
| SYPL1 | NM_182715.1 | 33239442 | ILMN_20394 | 5890202 | 0.0055 | 1.37 | 1.25 | 1.51 |
| DCUN1D4 | NM_015115.1 | 32698693 | ILMN_9395 | 610010 | 0.0055 | −1.18 | −1.23 | −1.13 |
| HCRTR2 | NM_001526.2 | 6006037 | ILMN_4206 | 6200736 | 0.0055 | 1.04 | 1.02 | 1.07 |
| STX5A | NM_003164.2 | 31543665 | ILMN_22175 | 6860288 | 0.0055 | 1.77 | 1.51 | 2.05 |
| CLEC4E | NM_014358.1 | 7657332 | ILMN_136933 | 940754 | 0.0055 | 1.77 | 1.51 | 2.05 |
| PSMA1 | NM_002786.2 | 23110933 | ILMN_2036 | 3130040 | 0.0054 | 1.82 | 1.59 | 2.10 |
| EVI2A | NM_001003927.1 | 51511748 | ILMN_29280 | 3990538 | 0.0054 | 1.34 | 1.24 | 1.46 |
| LOC651076 | XM_940198.1 | 89057421 | ILMN_46930 | 4220138 | 0.0054 | 1.34 | 1.24 | 1.45 |
| CDC42SE2 | NM_020240.1 | 9910377 | ILMN_138762 | 4250682 | 0.0054 | 2.59 | 2.10 | 3.14 |
| CLASP2 | NM_015097.1 | 57863300 | ILMN_25670 | 4780070 | 0.0054 | 1.50 | 1.34 | 1.68 |
| MAGED1 | NM_001005332.1 | 52632378 | ILMN_27182 | 6110086 | 0.0054 | 1.21 | 1.16 | 1.27 |
| RASGRP4 | NM_170604.1 | 26051257 | ILMN_17558 | 6200021 | 0.0054 | 2.00 | 1.71 | 2.32 |
| AGT | NM_000029.2 | 73622269 | ILMN_1261 | 6380273 | 0.0054 | 1.01 | −1.01 | 1.04 |
| HS.291319 | CR627122 | 50949744 | ILMN_85013 | 6980537 | 0.0054 | −1.50 | −1.73 | −1.31 |
| PDCD8 | NM_004208.2 | 22202627 | ILMN_20381 | 7320433 | 0.0054 | 1.32 | 1.25 | 1.40 |
| LOC649679 | XM_945045.1 | 88981262 | ILMN_34833 | 840433 | 0.0054 | −1.13 | −1.18 | −1.08 |
| CHKB | NM_005198.3 | 23238259 | ILMN_1067 | 2470592 | 0.0053 | 1.67 | 1.42 | 1.95 |
| STK16 | NM_001008910.1 | 57165435 | ILMN_23507 | 2640066 | 0.0053 | 1.27 | 1.20 | 1.34 |
| C19ORF6 | NM_001033026.1 | 74229024 | ILMN_12941 | 2900128 | 0.0053 | 1.46 | 1.31 | 1.61 |
| C6ORF25 | NM_138275.1 | 19913380 | ILMN_20734 | 3440161 | 0.0053 | 1.27 | 1.21 | 1.35 |
| LOC400197 | XM_928858.1 | 89037276 | ILMN_37870 | 3930112 | 0.0053 | 1.80 | 1.57 | 2.05 |
| ABLIM1 | NM_006720.3 | 51173716 | ILMN_21737 | 4570445 | 0.0053 | 1.60 | 1.42 | 1.78 |
| PDPK1 | NM_002613.3 | 60498971 | ILMN_27765 | 4850471 | 0.0053 | 2.15 | 1.81 | 2.56 |
| TMPO | NM_003276.1 | 4507554 | ILMN_12700 | 6590221 | 0.0053 | 1.47 | 1.32 | 1.63 |
| HS.579980 | CR984787 | 68223121 | ILMN_132161 | 7210358 | 0.0053 | 1.33 | 1.22 | 1.44 |
| THAP1 | NM_018105.2 | 40068498 | ILMN_15754 | 2600167 | 0.0052 | 1.91 | 1.64 | 2.21 |
| AMACR | NM_014324.4 | 42794624 | ILMN_3438 | 270603 | 0.0052 | 1.55 | 1.41 | 1.72 |
| C14ORF118 | NM_017926.2 | 40018645 | ILMN_23576 | 2900167 | 0.0052 | 1.33 | 1.23 | 1.43 |
| BRMS1 | NM_001024958.1 | 68348703 | ILMN_18543 | 3800730 | 0.0052 | 2.15 | 1.82 | 2.51 |
| TM9SF1 | NM_001014842.1 | 62460634 | ILMN_1371 | 3840491 | 0.0052 | 1.89 | 1.64 | 2.16 |
| C17ORF55 | NM_178519.2 | 31341837 | ILMN_17830 | 4880367 | 0.0052 | 1.17 | 1.12 | 1.24 |
| DYRK2 | NM_006482.1 | 5922003 | ILMN_18934 | 5270446 | 0.0052 | 1.08 | 1.05 | 1.10 |
| MR1 | NM_001531.1 | 4504416 | ILMN_10108 | 5310274 | 0.0052 | 1.91 | 1.62 | 2.22 |
| CD163 | NM_203416.1 | 44889962 | ILMN_17347 | 5570414 | 0.0052 | 1.77 | 1.50 | 2.08 |
| LOC642269 | XM_930699.1 | 89028396 | ILMN_30585 | 6060372 | 0.0052 | 1.28 | 1.20 | 1.37 |
| COP1 | NM_052889.2 | 62953111 | ILMN_21555 | 6100010 | 0.0052 | −1.34 | −1.46 | −1.22 |
| DDX17 | NM_006386.3 | 38201709 | ILMN_28983 | 6220035 | 0.0052 | 1.58 | 1.42 | 1.77 |
| HS.578712 | BG427758 | 13334264 | ILMN_130893 | 7200619 | 0.0052 | 1.30 | 1.21 | 1.39 |
| LOC90379 | XM_944706.1 | 89057238 | ILMN_34089 | 840452 | 0.0052 | 1.27 | 1.20 | 1.34 |
| PHF6 | NM_032335.2 | 63478059 | ILMN_21948 | 840520 | 0.0052 | 1.19 | 1.13 | 1.26 |
| FAM18B2 | XM_936923.1 | 89065553 | ILMN_137075 | 1230386 | 0.0051 | 1.77 | 1.54 | 2.03 |
| KCNH1 | NM_002238.2 | 27436999 | ILMN_6368 | 2030181 | 0.0051 | −1.06 | −1.09 | −1.02 |
| HS.561411 | CN364852 | 47364786 | ILMN_114851 | 3140241 | 0.0051 | −1.36 | −1.47 | −1.25 |
| CXORF15 | NM_018360.1 | 8922939 | ILMN_26850 | 3360592 | 0.0051 | 1.21 | 1.14 | 1.27 |
| SPCS3 | NM_021928.1 | 11345461 | ILMN_14718 | 5130255 | 0.0051 | 1.79 | 1.55 | 2.04 |
| LOC641848 | XM_935588.1 | 89027387 | ILMN_45490 | 5290070 | 0.0051 | −1.88 | −2.21 | −1.59 |
| PLAA | NM_001031689.1 | 72534669 | ILMN_14096 | 5310070 | 0.0051 | 1.19 | 1.15 | 1.24 |
| PHF17 | NM_199320.1 | 40556392 | ILMN_26400 | 5310152 | 0.0051 | 1.51 | 1.35 | 1.68 |
| ZNF124 | NM_003431.2 | 42733607 | ILMN_19934 | 7050474 | 0.0051 | 1.21 | 1.15 | 1.28 |
| NFRKB | NM_006165.2 | 23346419 | ILMN_18461 | 7560372 | 0.0051 | 1.30 | 1.21 | 1.40 |
| HS.432352 | BX113158 | 27838052 | ILMN_90908 | 1240204 | 0.005 | 1.09 | 1.05 | 1.12 |
| LOC651633 | XM_940830.1 | 89062068 | ILMN_39811 | 1510176 | 0.005 | −1.30 | −1.39 | −1.20 |
| HMGB1 | NM_002128.3 | 31982879 | ILMN_23421 | 2230367 | 0.005 | −1.23 | −1.30 | −1.16 |
| LOC653972 | XM_938779.1 | 89038888 | ILMN_31111 | 2510554 | 0.005 | 1.40 | 1.27 | 1.55 |
| ANAPC7 | NM_016238.1 | 7705283 | ILMN_4717 | 3440278 | 0.005 | 1.39 | 1.28 | 1.51 |
| CKAP5 | NM_001008938.1 | 57164941 | ILMN_12487 | 4280246 | 0.005 | 1.14 | 1.10 | 1.19 |
| HS.580128 | DA861647 | 82131639 | ILMN_132309 | 430181 | 0.005 | 1.27 | 1.19 | 1.36 |
| LOC650224 | XM_939316.1 | 89036235 | ILMN_34466 | 4480181 | 0.005 | 1.18 | 1.12 | 1.24 |
| ZNF200 | NM_003454.2 | 37675272 | ILMN_25695 | 4560672 | 0.005 | 1.16 | 1.12 | 1.20 |
| GCET2 | NM_001008756.1 | 57165368 | ILMN_21048 | 4730328 | 0.005 | 1.18 | 1.12 | 1.25 |
| LOC648998 | XM_938078.1 | 89065846 | ILMN_32035 | 4810543 | 0.005 | 1.42 | 1.30 | 1.55 |
| LOC647596 | XM_936646.1 | 89060867 | ILMN_35176 | 4900731 | 0.005 | 1.15 | 1.12 | 1.19 |
| DDX47 | NM_016355.3 | 41327774 | ILMN_8096 | 5310431 | 0.005 | 1.96 | 1.66 | 2.31 |
| CTNS | NM_004937.1 | 4826681 | ILMN_11769 | 5390273 | 0.005 | 1.33 | 1.25 | 1.41 |
| LOC129607 | NM_207315.1 | 46409273 | ILMN_3648 | 5720438 | 0.005 | 1.42 | 1.29 | 1.58 |
| HIST2H4 | NM_003548.2 | 29553982 | ILMN_22069 | 610300 | 0.005 | 1.88 | 1.62 | 2.20 |
| ZNF658 | NM_033160.4 | 55769536 | ILMN_14759 | 6770543 | 0.005 | −1.16 | −1.22 | −1.11 |
| C12ORF23 | NM_152261.1 | 22748614 | ILMN_11109 | 7040753 | 0.005 | −1.25 | −1.32 | −1.17 |
| BAD | NM_032989.1 | 14670387 | ILMN_27816 | 770739 | 0.005 | 1.22 | 1.16 | 1.29 |
| BTN3A3 | NM_006994.3 | 37574626 | ILMN_20620 | 160446 | 0.0049 | 2.54 | 2.01 | 3.16 |
| CSNK1G1 | NM_001011664.2 | 71773653 | ILMN_19857 | 2190056 | 0.0049 | 1.13 | 1.10 | 1.17 |
| VEGFB | NM_003377.3 | 39725673 | ILMN_15862 | 2350739 | 0.0049 | −1.11 | −1.21 | −1.02 |
| WDSOF1 | NM_015420.4 | 31542525 | ILMN_7024 | 3800170 | 0.0049 | −1.25 | −1.34 | −1.16 |
| LOC649419 | XM_941569.1 | 89036024 | ILMN_43489 | 3850100 | 0.0049 | 2.57 | 2.06 | 3.20 |
| C15ORF44 | XM_940546.1 | 89039133 | ILMN_138325 | 4610050 | 0.0049 | 1.71 | 1.51 | 1.92 |
| RUSC1 | NM_014328.2 | 42476122 | ILMN_13485 | 4780411 | 0.0049 | 1.50 | 1.32 | 1.69 |
| HIST1H2AC | NM_003512.3 | 21396481 | ILMN_26493 | 4890192 | 0.0049 | 2.45 | 1.90 | 3.14 |
| DPY19L3 | NM_207325.1 | 46409291 | ILMN_17111 | 50278 | 0.0049 | 1.22 | 1.15 | 1.28 |
| ACBD5 | NM_145698.1 | 21735486 | ILMN_12634 | 5360112 | 0.0049 | 1.41 | 1.29 | 1.55 |
| JAGN1 | NM_032492.2 | 31982910 | ILMN_2462 | 5360348 | 0.0049 | 1.35 | 1.24 | 1.47 |
| SPTLC1 | NM_006415.2 | 30474867 | ILMN_10107 | 5490768 | 0.0049 | 1.99 | 1.70 | 2.31 |
| LOC645472 | XM_928498.1 | 89050811 | ILMN_30737 | 5810154 | 0.0049 | −1.19 | −1.26 | −1.13 |
| DPP3 | NM_005700.2 | 18491023 | ILMN_138296 | 6370541 | 0.0049 | 1.35 | 1.26 | 1.44 |
| LOC440732 | XM_496441.2 | 88943885 | ILMN_38370 | 6550709 | 0.0049 | −1.76 | −2.08 | −1.45 |
| LOC644096 | XM_927323.1 | 89056790 | ILMN_41860 | 6660474 | 0.0049 | 1.28 | 1.19 | 1.37 |
| MBD2 | NM_015832.3 | 48255922 | ILMN_13743 | 7560255 | 0.0049 | 1.61 | 1.42 | 1.81 |
| HS.557625 | AW132136 | 6133743 | ILMN_112916 | 1110068 | 0.0048 | 1.08 | 1.05 | 1.13 |
| HS.557745 | AW138070 | 6142388 | ILMN_112965 | 1710204 | 0.0048 | 1.22 | 1.14 | 1.29 |
| HS.553605 | DN831967 | 62640651 | ILMN_111520 | 2070544 | 0.0048 | 1.27 | 1.19 | 1.37 |
| SEC24B | NM_006323.1 | 5454045 | ILMN_13898 | 2490520 | 0.0048 | 1.69 | 1.50 | 1.90 |
| PHTF2 | NM_020432.2 | 40254932 | ILMN_13666 | 2900438 | 0.0048 | −1.46 | −1.64 | −1.28 |
| LOC643995 | XM_930156.1 | 89042169 | ILMN_31229 | 430066 | 0.0048 | 1.29 | 1.18 | 1.42 |
| TLR4 | NM_138557.1 | 19924152 | ILMN_1390 | 4390615 | 0.0048 | 2.08 | 1.68 | 2.56 |
| FLJ11016 | NM_018301.2 | 38454187 | ILMN_16421 | 4860735 | 0.0048 | 1.29 | 1.20 | 1.38 |
| LOC644134 | XM_932013.1 | 89025548 | ILMN_35992 | 4880369 | 0.0048 | 1.31 | 1.22 | 1.39 |
| LOC402573 | NM_001004323.1 | 51972225 | ILMN_9570 | 4880709 | 0.0048 | 1.22 | 1.13 | 1.31 |
| NFATC2IP | XM_944125.1 | 89040767 | ILMN_137710 | 5960356 | 0.0048 | 1.28 | 1.18 | 1.39 |
| SRM | NM_003132.2 | 63253297 | ILMN_2445 | 6510725 | 0.0048 | −1.25 | −1.36 | −1.15 |
| DLEU1 | XR_001515.1 | 89036588 | ILMN_34088 | 6520274 | 0.0048 | −1.15 | −1.21 | −1.10 |
| SACM1L | NM_014016.2 | 41281578 | ILMN_19838 | 7040768 | 0.0048 | −1.48 | −1.72 | −1.28 |
| SEPT10 | NM_144710.2 | 30795194 | ILMN_5056 | 7400392 | 0.0048 | 1.09 | 1.06 | 1.12 |
| HSPBP1 | XM_938008.1 | 89057639 | ILMN_138021 | 7550736 | 0.0048 | 1.18 | 1.13 | 1.24 |
| HS.545128 | R07429 | 759352 | ILMN_108408 | 7560161 | 0.0048 | 1.18 | 1.12 | 1.25 |
| DEDD | NM_004216.2 | 14670395 | ILMN_13705 | 780709 | 0.0048 | 1.36 | 1.24 | 1.48 |
| CBWD3 | NM_201453.1 | 42558280 | ILMN_18690 | 130180 | 0.0047 | −1.05 | −1.08 | −1.02 |
| ASPSCR1 | XM_941362.1 | 89043116 | ILMN_138126 | 2690112 | 0.0047 | 1.40 | 1.29 | 1.52 |
| SBNO1 | NM_018183.2 | 33620762 | ILMN_12636 | 3610041 | 0.0047 | 1.47 | 1.35 | 1.60 |
| HS.545727 | AA668234 | 2629733 | ILMN_108864 | 4860500 | 0.0047 | 1.05 | 1.02 | 1.08 |
| HIPK3 | NM_005734.2 | 29469068 | ILMN_20690 | 6650301 | 0.0047 | 1.62 | 1.46 | 1.78 |
| ANKRD13D | XM_945567.1 | 89034918 | ILMN_138354 | 2470189 | 0.0046 | 1.17 | 1.12 | 1.23 |
| LOC390414 | XM_940915.1 | 89031778 | ILMN_42621 | 3420458 | 0.0046 | −1.10 | −1.14 | −1.07 |
| RFXDC1 | NM_173560.1 | 27734870 | ILMN_10052 | 3440053 | 0.0046 | −1.05 | −1.07 | −1.03 |
| HDAC9 | NM_058177.1 | 17158040 | ILMN_20565 | 5050634 | 0.0046 | 1.24 | 1.18 | 1.30 |
| MBTPS1 | NM_003791.2 | 41350325 | ILMN_12720 | 5310037 | 0.0046 | 1.67 | 1.48 | 1.88 |
| HS.379327 | CX165253 | 56795333 | ILMN_88679 | 540142 | 0.0046 | 1.52 | 1.36 | 1.68 |
| HS.578208 | AA431122 | 2114830 | ILMN_130389 | 6480138 | 0.0046 | 1.22 | 1.15 | 1.29 |
| SLC26A2 | NM_000112.2 | 45935386 | ILMN_21352 | 6480692 | 0.0046 | 1.28 | 1.17 | 1.38 |
| HS.581533 | AA431235 | 2114943 | ILMN_133714 | 6550373 | 0.0046 | 1.07 | 1.04 | 1.11 |
| TUBG1 | NM_001070.3 | 34222287 | ILMN_1608 | 6770553 | 0.0046 | 1.15 | 1.10 | 1.19 |
| P2RX5 | NM_175081.1 | 28416936 | ILMN_10544 | 730040 | 0.0046 | 1.38 | 1.24 | 1.52 |
| LOC641808 | XM_935566.1 | 89027344 | ILMN_43943 | 830541 | 0.0046 | 1.26 | 1.18 | 1.34 |
| AFTIPHILIN | NM_203437.2 | 50409938 | ILMN_16341 | 2230392 | 0.0045 | −1.62 | −1.83 | −1.43 |
| EXOSC1 | NM_016046.2 | 22035626 | ILMN_138117 | 2360433 | 0.0045 | 1.33 | 1.23 | 1.43 |
| MDS1 | NM_004991.1 | 4826827 | ILMN_9694 | 240332 | 0.0045 | 1.21 | 1.15 | 1.28 |
| HS.247659 | BI752029 | 15743607 | ILMN_83183 | 3930450 | 0.0045 | 1.10 | 1.07 | 1.13 |
| HS.201113 | BX108670 | 27835318 | ILMN_81638 | 4560328 | 0.0045 | 1.33 | 1.22 | 1.45 |
| GPR177 | NM_024911.4 | 50541968 | ILMN_3521 | 4760309 | 0.0045 | 1.51 | 1.34 | 1.70 |
| CBX3 | NM_016587.2 | 20544150 | ILMN_11642 | 4880020 | 0.0045 | 1.61 | 1.42 | 1.83 |
| ANKRD13D | XM_945565.1 | 89034916 | ILMN_138345 | 5670091 | 0.0045 | 1.13 | 1.08 | 1.18 |
| FLJ45187 | NM_207371.2 | 50726976 | ILMN_5232 | 6350528 | 0.0045 | 1.08 | 1.05 | 1.11 |
| HS.291377 | CN430296 | 47417890 | ILMN_85018 | 6420288 | 0.0045 | −1.12 | −1.17 | −1.09 |
| HNRPA1 | NM_002136.1 | 4504444 | ILMN_137048 | 6620292 | 0.0045 | 1.39 | 1.29 | 1.50 |
| SEPT11 | NM_018243.2 | 38605734 | ILMN_27161 | 7380670 | 0.0045 | −1.34 | −1.47 | −1.21 |
| HS.577425 | DB337747 | 83130755 | ILMN_129606 | 830368 | 0.0045 | 1.42 | 1.28 | 1.57 |
| LOC644122 | XM_934731.1 | 89040142 | ILMN_46404 | 1070707 | 0.0044 | 1.89 | 1.63 | 2.17 |
| LOC644380 | XM_929628.1 | 89058831 | ILMN_36938 | 2060358 | 0.0044 | −1.21 | −1.28 | −1.15 |
| BCR | NM_021574.1 | 11038640 | ILMN_136985 | 4050427 | 0.0044 | −1.17 | −1.26 | −1.09 |
| SNRPN | NM_003097.3 | 29540556 | ILMN_15998 | 4060195 | 0.0044 | 2.17 | 1.77 | 2.63 |
| LOC646426 | XM_929353.1 | 89030901 | ILMN_45139 | 4540296 | 0.0044 | 1.19 | 1.14 | 1.25 |
| PPM1A | NM_177951.1 | 29557854 | ILMN_13918 | 5340066 | 0.0044 | 1.37 | 1.26 | 1.48 |
| GCH1 | NM_000161.2 | 66932966 | ILMN_2599 | 5550767 | 0.0044 | 1.41 | 1.31 | 1.52 |
| C10ORF61 | NM_001013840.1 | 62079296 | ILMN_24822 | 6270546 | 0.0044 | 1.56 | 1.39 | 1.74 |
| MAX | NM_145113.1 | 21704264 | ILMN_2124 | 6330180 | 0.0044 | 1.83 | 1.61 | 2.08 |
| METRNL | NM_001004431.1 | 52345386 | ILMN_13002 | 6480026 | 0.0044 | 1.77 | 1.54 | 2.05 |
| NOLA1 | NM_018983.2 | 15011914 | ILMN_138472 | 1430309 | 0.0043 | 1.71 | 1.49 | 1.94 |
| HS.574749 | AA701948 | 2705061 | ILMN_126930 | 1780019 | 0.0043 | 1.01 | −1.03 | 1.05 |
| LOC644823 | XM_932416.1 | 89025127 | ILMN_37724 | 2690669 | 0.0043 | 1.19 | 1.14 | 1.25 |
| ATE1 | NM_007041.2 | 50345874 | ILMN_26196 | 2850543 | 0.0043 | 1.27 | 1.19 | 1.35 |
| KCNV2 | NM_133497.2 | 28329446 | ILMN_23743 | 3870040 | 0.0043 | 1.32 | 1.23 | 1.41 |
| LOC653371 | XM_927125.1 | 88983818 | ILMN_36293 | 4150431 | 0.0043 | 2.30 | 1.84 | 2.83 |
| ST8SIA4 | NM_175052.1 | 28373098 | ILMN_7432 | 4590367 | 0.0043 | 2.47 | 1.96 | 3.06 |
| ZNF628 | NM_033113.1 | 60097911 | ILMN_6417 | 5260377 | 0.0043 | −1.05 | −1.08 | −1.02 |
| LOC652314 | XM_941733.1 | 88955139 | ILMN_35193 | 5260619 | 0.0043 | 1.07 | 1.03 | 1.11 |
| HS.569049 | R79598 | 855879 | ILMN_121230 | 5310273 | 0.0043 | 1.01 | −1.02 | 1.04 |
| LOC643319 | XM_927980.1 | 89028240 | ILMN_41137 | 1440427 | 0.0042 | 2.89 | 2.21 | 3.72 |
| SLC6A6 | NM_003043.2 | 54607093 | ILMN_25763 | 3520086 | 0.0042 | 1.56 | 1.38 | 1.77 |
| HS.127242 | CR607514 | 50488321 | ILMN_76396 | 4150192 | 0.0042 | 1.49 | 1.34 | 1.67 |
| KRTAP19-5 | NM_181611.1 | 31791021 | ILMN_1096 | 6130181 | 0.0042 | −1.04 | −1.07 | −1.01 |
| C14ORF126 | NM_080664.1 | 18087836 | ILMN_138867 | 6350768 | 0.0042 | 1.33 | 1.23 | 1.43 |
| ARSD | NM_009589.2 | 71852585 | ILMN_23380 | 670605 | 0.0042 | 1.21 | 1.15 | 1.27 |
| CECR1 | NM_177405.1 | 29029551 | ILMN_29872 | 1570468 | 0.0041 | 1.16 | 1.10 | 1.22 |
| ANKRD17 | NM_032217.3 | 38683806 | ILMN_11277 | 160278 | 0.0041 | 1.17 | 1.12 | 1.22 |
| PCNA | NM_182649.1 | 33239450 | ILMN_6858 | 3140403 | 0.0041 | 1.26 | 1.19 | 1.33 |
| HS.192268 | AI216576 | 3785617 | ILMN_81161 | 3990471 | 0.0041 | 1.23 | 1.14 | 1.32 |
| THRAP5 | NM_005481.2 | 38146093 | ILMN_24784 | 3990768 | 0.0041 | 1.86 | 1.58 | 2.15 |
| HS.253430 | AW204748 | 6504220 | ILMN_83478 | 4120441 | 0.0041 | −1.09 | −1.12 | −1.06 |
| LOC646082 | XM_929042.1 | 89058924 | ILMN_39807 | 4490167 | 0.0041 | −1.13 | −1.18 | −1.07 |
| DKFZP781I1119 | NM_152622.2 | 40255126 | ILMN_20832 | 6020487 | 0.0041 | 1.11 | 1.07 | 1.14 |
| SLC39A6 | NM_012319.2 | 12751474 | ILMN_3197 | 6420026 | 0.0041 | 1.92 | 1.67 | 2.20 |
| BCDO2 | NM_031938.2 | 41350209 | ILMN_138224 | 6770615 | 0.0041 | 1.40 | 1.27 | 1.52 |
| CROP | NM_006107.2 | 52426742 | ILMN_10300 | 1570670 | 0.004 | −1.04 | −1.06 | −1.01 |
| UEV3 | NM_018314.2 | 23943813 | ILMN_10227 | 4760196 | 0.004 | 1.30 | 1.20 | 1.40 |
| SEC24B | XM_945425.1 | 88980515 | ILMN_138457 | 5690674 | 0.004 | 1.72 | 1.50 | 1.94 |
| HS.548785 | BF062138 | 10821048 | ILMN_109905 | 6480577 | 0.004 | 1.04 | 1.01 | 1.06 |
| TENC1 | NM_015319.2 | 38787940 | ILMN_3159 | 1690711 | 0.0039 | 1.01 | −1.02 | 1.03 |
| SMARCD1 | NM_003076.3 | 21264349 | ILMN_16093 | 2710193 | 0.0039 | −1.11 | −1.16 | −1.07 |
| AFG3L1 | NM_001031805.1 | 73476314 | ILMN_11411 | 5220301 | 0.0039 | 1.09 | 1.06 | 1.12 |
| LOC652608 | XM_942140.1 | 89071890 | ILMN_39273 | 5720730 | 0.0039 | −1.21 | −1.28 | −1.15 |
| HS.570989 | BQ420825 | 21116140 | ILMN_123170 | 6060296 | 0.0039 | 1.29 | 1.19 | 1.41 |
| HIVEP2 | NM_006734.2 | 19923373 | ILMN_21520 | 6380546 | 0.0039 | −1.20 | −1.32 | −1.10 |
| PLS1 | NM_002670.1 | 4505896 | ILMN_26859 | 1710551 | 0.0038 | −1.05 | −1.07 | −1.03 |
| ADAM15 | XM_937888.1 | 88952487 | ILMN_138255 | 3990709 | 0.0038 | 1.22 | 1.16 | 1.30 |
| HS.568712 | DA188950 | 80506383 | ILMN_120893 | 5310333 | 0.0038 | 1.21 | 1.14 | 1.28 |
| HS.580148 | DB338928 | 83154923 | ILMN_132329 | 5390672 | 0.0038 | 1.37 | 1.26 | 1.49 |
| SMAP1L | NM_022733.1 | 23943871 | ILMN_1697 | 1010168 | 0.0037 | 1.76 | 1.47 | 2.10 |
| LOC653125 | XM_931236.1 | 89038164 | ILMN_38938 | 380544 | 0.0037 | 1.18 | 1.12 | 1.24 |
| LDHB | NM_002300.3 | 22726178 | ILMN_16800 | 4040609 | 0.0037 | −1.71 | −2.03 | −1.43 |
| HS.570348 | AI199741 | 3752347 | ILMN_122529 | 4250176 | 0.0037 | −1.02 | −1.05 | 1.01 |
| SOS1 | NM_005633.2 | 15529995 | ILMN_11376 | 5720719 | 0.0037 | 1.39 | 1.24 | 1.53 |
| AGPAT7 | NM_153613.1 | 23957707 | ILMN_137968 | 6350427 | 0.0037 | −1.13 | −1.19 | −1.07 |
| HS.125087 | BQ437417 | 21176493 | ILMN_76085 | 6900603 | 0.0037 | 1.30 | 1.20 | 1.41 |
| LOC643007 | XM_927198.1 | 89038191 | ILMN_39863 | 1110524 | 0.0036 | −1.64 | −1.90 | −1.41 |
| HS.560740 | BQ775960 | 21984436 | ILMN_114429 | 1400164 | 0.0036 | 1.04 | 1.02 | 1.06 |
| HS.574780 | BG198379 | 13720066 | ILMN_126961 | 4150228 | 0.0036 | 1.35 | 1.25 | 1.46 |
| EIF1AX | NM_001412.3 | 77404356 | ILMN_22164 | 4610546 | 0.0036 | −1.41 | −1.55 | −1.27 |
| SACS | NM_014363.3 | 38230497 | ILMN_3633 | 4780400 | 0.0036 | −1.12 | −1.16 | −1.09 |
| LOC400807 | XM_933808.1 | 88986393 | ILMN_32838 | 5670551 | 0.0036 | 1.18 | 1.12 | 1.23 |
| LOC284361 | NM_175063.3 | 45580693 | ILMN_139159 | 670369 | 0.0036 | −1.14 | −1.19 | −1.09 |
| APOBEC3A | NM_145699.2 | 22907036 | ILMN_12846 | 2810040 | 0.0035 | −1.72 | −1.99 | −1.48 |
| NEDD1 | NM_152905.2 | 34303960 | ILMN_4251 | 4610132 | 0.0035 | 1.39 | 1.28 | 1.52 |
| HS.547807 | BQ888875 | 22280889 | ILMN_109650 | 4640575 | 0.0035 | 1.39 | 1.28 | 1.53 |
| HS.163416 | CR745073 | 51667560 | ILMN_79907 | 7570722 | 0.0035 | −1.01 | −1.04 | 1.01 |
| AAAS | NM_015665.3 | 34222322 | ILMN_22994 | 870088 | 0.0035 | −1.13 | −1.18 | −1.07 |
| APPBP1 | NM_001018159.1 | 66363685 | ILMN_19510 | 4060465 | 0.0034 | 1.15 | 1.08 | 1.22 |
| LOC643550 | XM_926853.1 | 89035757 | ILMN_35341 | 4120458 | 0.0034 | −1.14 | −1.19 | −1.09 |
| CEP72 | NM_018140.2 | 62899064 | ILMN_10995 | 430279 | 0.0034 | −1.06 | −1.11 | −1.02 |
| SNAP23 | NM_130798.1 | 18765730 | ILMN_679 | 4880390 | 0.0034 | −1.68 | −1.97 | −1.42 |
| HS.570330 | AW962683 | 8152519 | ILMN_122511 | 7000300 | 0.0034 | 1.12 | 1.08 | 1.16 |
| PNLIPRP2 | NM_005396.3 | 37059783 | ILMN_9007 | 7210465 | 0.0034 | 1.06 | 1.03 | 1.10 |
| FAM11A | NM_032508.1 | 22296883 | ILMN_24307 | 1450750 | 0.0033 | 1.43 | 1.30 | 1.56 |
| HS.574453 | AK024399 | 10436778 | ILMN_126634 | 1710398 | 0.0033 | −1.03 | −1.06 | −1.01 |
| ASAH1 | NM_004315.2 | 30089929 | ILMN_27657 | 2030010 | 0.0033 | 1.08 | 1.04 | 1.11 |
| ASB15 | NM_080928.2 | 38261966 | ILMN_8926 | 2060132 | 0.0033 | −1.06 | −1.08 | −1.04 |
| HS.582091 | DA326910 | 78741011 | ILMN_134272 | 3130176 | 0.0033 | −1.09 | −1.11 | −1.06 |
| SCRIB | NM_182706.2 | 45827730 | ILMN_21867 | 3800470 | 0.0033 | −1.04 | −1.07 | −1.01 |
| HS.552431 | AA579194 | 2357378 | ILMN_110992 | 4060142 | 0.0033 | 1.10 | 1.03 | 1.16 |
| PPHLN1 | NM_016488.5 | 48255928 | ILMN_6863 | 4120259 | 0.0033 | −1.12 | −1.16 | −1.08 |
| MGC40499 | XM_941945.1 | 89026172 | ILMN_139128 | 4780487 | 0.0033 | 1.23 | 1.16 | 1.30 |
| KIAA0423 | NM_015091.1 | 44888819 | ILMN_18327 | 6100692 | 0.0033 | −1.30 | −1.41 | −1.20 |
| HRIHFB2122 | NM_138632.1 | 20336762 | ILMN_138238 | 630669 | 0.0033 | 1.31 | 1.24 | 1.39 |
| UBE2D3 | NM_181889.1 | 33149315 | ILMN_28535 | 7200097 | 0.0033 | 1.15 | 1.11 | 1.19 |
| SLC27A6 | NM_014031.3 | 62865629 | ILMN_10102 | 7550131 | 0.0033 | 1.42 | 1.30 | 1.55 |
| GSR | NM_000637.2 | 50301237 | ILMN_14467 | 7560093 | 0.0033 | 1.79 | 1.55 | 2.05 |
| FBXO43 | NM_001029860.1 | 71143129 | ILMN_7498 | 2900669 | 0.0032 | 1.08 | 1.05 | 1.11 |

*For each covariate entry the United States National Center for Biotechnology Information (NCBI, U.S. National Library of Medicine, 800 Rockville Pike, Bethesda, MD, 20894 USA) identifiers (accession number/version and NCBI GI Number) are provided. Those NCBI identifiers uniquely identify nucleic acid and/or protein sequences present in the NCBI databases and are publicly available, for example, on the word wide web at www.ncbi.nlm.nih.gov. Where an NCBI accession number or GI number is provided for a nucleic acid sequence encoding a protein produced by a gene indicated herein (e.g., a cDNA sequence) the corresponding gene sequence is also available in the NCBI database and considered part of this disclosure. Where any accession number does not recite a specific version, the version is taken to be the most recent version of the sequence associated with that accession number at the time the earliest priority document for the present application was filed.
NA = Not Applicable

Supplementary Table III
Posterior probabilities from the spirometric class, and random
forest model-predicted class for the 16 misclassified
subjects in the test set (n = 65, FEV₁/FVC 0.60-0.75).

| | | | Random forest |
|---|---|---|---|
| P (Control\|RF) | P (Case\|RF) | Spirometric class | model-predicted class |
| 0.517 | 0.483 | Case | Control |
| 0.684 | 0.316 | Case | Control |
| 0.886 | 0.114 | Case | Control |
| 0.912 | 0.088 | Case | Control |
| 0.925 | 0.075 | Case | Control |
| 0.912 | 0.088 | Case | Control |
| 0.936 | 0.064 | Case | Control |
| 0.641 | 0.359 | Case | Control |
| 0.821 | 0.180 | Case | Control |
| 0.860 | 0.140 | Case | Control |
| 0.894 | 0.106 | Case | Control |
| 0.572 | 0.428 | Case | Control |
| 0.606 | 0.394 | Case | Control |
| 0.955 | 0.046 | Case | Control |
| 0.042 | 0.958 | Control | Case |
| 0.477 | 0.528 | Control | Case |

FEV₁, forced expiratory volume in 1 s;
FVC, forced vital capacity

Substitutions, modifications, changes and omissions may be made in the design, operating conditions and arrangement of the aspects and embodiments described herein without departing from the spirit of the subject matter as expressed, inter alia, in the appended claims. Additional advantages, features and modifications will readily occur to those skilled in the art. Therefore, the subject matter of this disclosure, in its broader aspects, is not limited to the specific details, examples, or representative devices, shown and described herein. Accordingly, various modifications may be made without departing from the spirit or scope of the general concepts as defined, inter alia, by the appended claims and their equivalents.

All of the references cited herein, including patents, patent applications, and publications, are hereby incorporated in their entireties by reference.

The scope of the claims below is not restricted to the particular embodiments described herein. The following examples describe for illustrative purposes and are not intended to limit the methods and compositions of the present disclosure in any manner. Those of skill in the art will recognize a variety of parameters that can be changed or modified to yield the same results.

1. A composition comprising two nucleic acid molecules, wherein the first nucleic acid molecule comprises a first nucleotide sequence and the second nucleic acid molecule comprises a second nucleotide sequence, wherein the first nucleotide sequence differs from the second nucleotide sequence and the first and second nucleotide sequences are selected independently from the group consisting of the sequences of the nucleic acids set forth in Supplementary Table II or a fragment of any thereof, and nucleotide sequences having 70-99% identity to the nucleic acid set forth in Supplementary Table II or a fragment of any thereof.

2. The composition of claim 1, further comprising a third nucleic acid molecule comprising a third nucleotide sequence, wherein the third nucleotide sequence differs from the first and the second nucleotide sequences and the third nucleotide sequence is selected independently from the group consisting of the sequences of the nucleic acids set forth in Supplementary Table II or a fragment of any thereof, and nucleotide sequences having 70-99% identity to the nucleic acid set forth in Supplementary Table II or a fragment of any thereof.

3-9. (canceled)

10. The composition of claim 2, wherein the first through the nth third nucleotide sequences each comprise a nucleotide sequence of a nucleic acid expressed by a gene selected from the group consisting of IL6R, CCR2, PPP2CB, RASSF2, WTAP, DNTTIP2, GDAP1, LIPE, and RPL14, or a sequence having 70%-99% identity to a nucleic acid expressed by a gene selected from the group consisting of IL6R, CCR2, PPP2CB, RASSF2, WTAP, DNTTIP2, GDAP1, LIPE, and RPL14, or a sequence complementary to any thereof, or a fragment of any of the foregoing.

11-25. (canceled)

26. A method of diagnosing lung disease or an increased risk of developing lung disease in a subject comprising measuring the expression of 2, 3, 4, 6, 8, 10, 12, 25, 20, 30, 40, 50, 75, 100, 200, 300, 400, 500, or more nucleic acids expressed from the nucleic acids set forth in Supplementary Table II or fragments thereof.

27. The method of claim 26, comprising measuring the expression of 2 or more nucleic acid molecules expressed by two or more genes selected from the group consisting of: IL6R, CCR2, PPP2CB, RASSF2, WTAP, DNTTIP2, GDAP1, LIPE, and RPL14.

28. (canceled)

29. The method of claim 27, wherein the lung disease is selected from the group consisting of asthma, chronic obstructive pulmonary disease (COPD), lung cancer, alpha-1 antitrypsin deficiency, respiratory distress syndrome, chronic bronchitis, chronic systemic inflammation, and inflammatory respiratory disease.

30. The method of claim 27, wherein the lung disease is COPD.

31. The method of claim 29, wherein increased nucleic acid expression correlates with a diagnosis of lung disease or an increased risk of developing lung disease.

32. The method of claim 30, wherein increased nucleic acid expression correlates with a diagnosis of lung disease or an increased risk of developing lung disease.

33-34. (canceled)

35. A method of diagnosing lung disease or an increased risk of developing lung disease in a subject comprising:
a. obtaining a measurement of the level of expression of one or more nucleic acids set forth in Supplementary Table II in a sample from a subject; and
b. comparing the measurement of the levels of expression in the sample from the subject to the level of expression of said one or more nucleic acids set forth in Supplementary Table II in a control sample;
wherein said control sample is obtained from an individual or population of individuals not having lung disease; and
wherein a difference in levels of expression in the sample from the subject as compared to the levels of expression in the control sample indicates that the subject has or is at risk of developing lung disease.

36. A method screening a subject who smokes tobacco products for the risk of developing lung disease or a decline in lung function comprising:
(a) obtaining a measurement of the level of expression of one or more nucleic acids set forth in Supplementary Table II in a sample from the subject; and
(b) comparing the measurement of the levels of expression in the sample from the subject to the level of expression of said one or more nucleic acids set forth in Supplementary Table II in a control sample; wherein said control sample is obtained from an individual or population of individuals not having lung disease; and wherein a difference in levels of expression in the sample from the subject as compared to the levels of expression in the control sample indicates that the subject has or is at risk of developing lung disease or a decline in lung function.

37-38. (canceled)

39. The method of claim 36, wherein the difference is an increased expression of any one, two, three, four or five of CCR2, IL6R, PP2CB, RASSF2 and WT AP and/or a decreased expression of any one, two, three, or four of DNTTIP2, GDAP1, LIPE, RPL 14.

40. (canceled)

41. A method of treating a subject having or suspected of having a lung disease or of following the course of lung disease in a subject having or suspected of having a lung disease comprising:
(a) obtaining a measurement of the level of expression of one or more nucleic acids set forth in Supplementary Table II in a sample from the subject at a first time; and
(b) obtaining a second measurement of the level of expression of at least the same one or more nucleic acids set forth in Supplementary Table II in a second sample obtained from the subject at a second time; and comparing the first measurement to the second measurement to determine the progression or regression or stability of the lung disease.

42. The method of claim 41, wherein at least one measurement is conducted by measuring or observing the quantity or concentration of one or more proteins encoded by a nucleic acid set forth in Supplementary Table II.

43. The method of any of claim 41, wherein at least one therapeutic agent is administered to said subject,
wherein said first sample was obtained from said subject before said second sample and said therapeutic agent is administered after said first sample was obtained from said subject, and before said second sample was obtained from said subject; and
wherein said therapeutic agent is selected from the group consisting of immunosuppressants, corticosteroids, p2(beta 2)-adrenergic receptor agonists, anticholinergics, and oxygen

44-45. (canceled)

46. The method of claim 41, further comprising changing the treatment of a subject based upon said progression or regression or stability of said lung disease.

47. A device comprising a plurality of locations, wherein 2, 3, 4, 5, 6, 7, 8 or more of said locations each comprise a different nucleic acid molecule having a nucleotide sequence of a nucleic acid molecule set forth in Supplementary Table II, or a sequence having 70-99% identity to the nucleic acid sequence of a nucleic acid molecule set forth in Supplementary Table II, or a fragment of any of the foregoing.

48. The device of claim 47, wherein said 2, 3, 4, 5, 6, 7, 8 or more of said locations comprise a nucleic acid molecule encoding a protein expressed from a different gene selected from CCR2, IL6R, PP2CB, RASSF2, WT AP, DNTTIP2, GDAP1, LIPE. and RPL14, or a sequence having 70-99% identity to a nucleic acid molecule encoding a protein expressed from a different gene selected from CCR2, IL6R, PP2CB, RASSF2, WTAP, DNTTIP2, GDAP1, LIPE, and RPL14, or a complement or fragment of any of the foregoing having a length from about 20 to about 225 nucleotides.

49-50. (canceled)


SEQUENCE LISTING (9 sequences)

SEQ ID NO: 1 - DNA, 4176 bp, Homo sapiens
ggcggtcccctgttctccccgctcaggtgcggcgctgtggcaggaagccaccccctcggtcggccggtgcgcggggctgttgcgccatccgctccggctttcgtaaccgcaccctgggacggcccagagacgctccagcgcgagttcctcaaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctggg

SEQ ID NO: 2 - DNA, 2556 bp, Homo sapiens
agaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgataaaagatt

SEQ ID NO: 3 - DNA, 1987 bp, Homo sapiens
ggaggggagagaaagagcgagagaaggggaaagacaagtcgggagaggccggtaggcgtgaggcgggcctgaagcggcagcgggcggccttcgtccggcgagagctaggccgaggacccgcgccgcgctccccggcacctcaccgcgtccttcaccgactcccgcggcgcgcggccgggcggggaagggcgggcgggggtctcctccaggctgcgcgctcggagccgcctgctgggcttgggcggggcgcggggcccgcggccgccctacccggctcagtcctccccctgtgggacctggcgacggcggcggagggagaggggagcggcgcccgggccggggccgggggcgggtggggaggggggagggcggcggccgggctggggctcgggatccgcatcgggatcgggccgccatggacgacaaggcgttcaccaaggagctggaccagtgggtcgagcagctgaacgagtgtaagcagctgaacgagaaccaagtgcggacgctgtgcgagaaggcaaaggaaattttaacaaaagaatcaaatgtgcaagaggttcgttgccctgttactgtctgtggagatgtgcatggtcaatttcatgatcttatggaactctttagaattggtggaaaatcaccggatacaaactacttattcatgggtgactatgtagacagaggatattattcagtggagactgtgactcttcttgtagcattaaaggtgcgttatccagaacgcattacaatattgagaggaaatcacgaaagccgacaaattacccaagtatatggcttttatgatgaatgtctgcgaaagtatgggaatgccaacgtttggaaatattttacagatctctttgattatcttccacttacagctttagtagatggacagatattctgcctccatggtggcctctctccatccatagacacactggatcatataagagccctggatcgtttacaggaagttccacatgagggcccaatgtgtgatctgttatggtcagatccagatgatcgtggtggatggggtatttcaccacgtggtgctggctacacatttggacaagacatttctgaaacctttaaccatgccaatggtctcacactggtttctcgtgcccaccagcttgtaatggagggatacaattggtgtcatgatcggaatgtggttaccattttcagtgcacccaattactgttatcgttgtgggaaccaggctgctatcatggaattagatgacactttaaaatattccttccttcaatttgacccagcgcctcgtcgtggtgagcctcatgttacacggcgcaccccagactacttcctataaatttctcctgggaaacctgcctttgtatgtggaagtatacctggctttttaaaatatatgtatttaaaaacaaaaagcaacagtaatctatgtgtttctgtaacaaattgggatctgtcttggcattaaaccacatcatggaccaaatgtgccatactaatgatgagcatttagcacaatttgagactgaaatttagtacactatgttctaggtcagtctaacagtttgcctgctgtatttatagtaaccattttcctttggactgttcaagcaaaaaaggtaactaactgcttcatctccttttgcgcttatttggaaattttagttatagtgtttaactggcatggattaatagagttggagttttatttttaagaaaaattcacaagctaacttccactaatccattatcctttattttattgaaatgtataattaacttaactgaagaaaaggttcttcttgggagtatgttgtcataacatttaaagagatttcccttcatttaaactaaattactgttttatgttgatctgcatatttctgtatatttgtcatgacagtgcttgcatcctatttggtgtactcagcaaataaacttttcattttaaacaaaaacattcaaaaaaaaaaaaaaaaaa

SEQ ID NO: 4 - DNA, 5223 bp, Homo sapiens
gtggctagtgaggcccagcctgtatatttctaacagggcgccccatgccccagcaccctccccactgggaccactcctcatagaatagccccactccaaagcatttatgacttgaaccttctgaccaaagctctctgaatcttgggggaacctagaagaggaaaaggaatgtcaggctctgtcagagtcagccaagcgagatcagccaggggtctgctccctgcagaccatacggggagccagggtttgcaatgagtctaaactggaatcttaccctgcaaaacgaatggccactattagagttttccaaaaccactctgaagcccctgactgtgcccaaagttcagatctcagaggtggatgccccgccggagggtgaccagatgccaagctccacagactccaggggcctgaagcccctgcaggaggacaccccacagctgatgcgcacacgcagtgatgttggggtgcgtcgccgtggcaatgtgaggacgcctagtgaccagcggcgaatcagacgccaccgcttctccatcaacggccatttctacaaccataagacatccgtgttcacaccagcctatggctctgtcaccaacgtccgcatcaacagcaccatgaccaccccacaggtcctgaagctgctgctcaacaaatttaagattgagaattcagcagaggagtttgccttgtacgtggtccatacgagtggtgggcccatgtgagcagatctccaaagtgttcctaatggagaaggaccaggtggaggaagtcacctacgacgtggcccagtatataaagttcgagatgccggtacttaaaagcttcattcagaagctccaggaggaagaagatcgggaagtaaagaagctgatgcgcaagtacaccgtgctccggctaatgattcgacagaggctggaggagatagccgagaccccagcaacaatctgagccatgagaacgaggggatctgggcaccccaggaaccgccattgcccataagacccccaggaagctaggcactttctttccatggaaacatttagacacaaacctccccagctccggccaagccatcatttgctacctggagctggatgtagaagtcagcagacagctccctatccctggacccctgccctccttttttctgctcacaaggacttttgattttagttataaggaggacccaaaatgtgtgtgtgtacatgtgtgtgcacacatggtacgtgtccatgtgcctacctgatactttcacatgtaattaaattccaggcaaccagcacaagagccgtgagcttggcacatgtgctgctcgtgagcaggaaaatcagaggagccactgatctgagtggtatttaggttgaaggaaagatttctcctctcaagtgccagggagcagccacacgtctgtctgtgtttagagagggaagagggttctccaggttcaccatttgggttgtttatatgttggtagaaattctccctgtatgcctagaaggatcagtgaatgtaagagccttggaaattaacaaaataacagccacataaccttgcggcaagtctgatggaaagaaaaagataaaccatccgtggggtagatgcaataagcccacgtatttttacactggaaacgttgattgttttaaatgacaaagacatatgtgatgttctatgtggaaacctgtgaagagtggattctgcctccatctctgcctccatggctacctttaggagacagagaagatcctgtgtgtttctctgtacccagctgacagcctgtctctatggcgcttccttgagtggaaggaaatgtctcaagaaacaaagatctcgctggtgcgtacacagtgctgaccagctagtgtggccagggcctggtggcctggtggccaggaagtttcaggttgaagggaaatgtcgaggctacctgcagatatgacaggtgccttgaacgcagcccatcttcatgtcatcaaaggtcttcctgcacttgaagctggggcgatgtttgcagtcaagaccattctttccaacctctgggttcttgcaagttgccctcaccttgtgtgtggagatgcattccaagaatgaagcctcatcttgctactgagtgtggggttcagggaagctctttaggccacctggtgaaggtgcatggggaggatggagcttctcctcagctcctctgagcagccacctatgtgatctttaaatccaaccccaatgggagaaaagggcaagaacagtctgtgccctgggactcctatcaggaagcttgacaggcagctgggcatcagtgcagctgatatcgtttgaggagggagacagatgcttggacctgggtgcctggctatggagattgaccaagcaagatcaggagctcctgatagcaggcgtctttgagcctagctggggtagaggcactgcccatctcttctccaccttctctccacagaatgtttgcagagctgggcagttgaggaaaggacagcccctggttggtgcctccaaaggaaggtggacttttttggtggagacgtttctgccctgggcaccctcctgcccccgattcatacctatggcttcttgagaaggctcacagctgtggtcttaacgtagactgcagaaagatggcatgcggcccctggcatttcgccaagggttttatagcaagtctccttcctccatagggacagcagcaccagccctgtggggcatggagtggaagcccagaagggcttctgcaagctgcacagaactggggtaagaagacaaagagtagccaccgggagaggcttcctttgttacagctgggaaagaacagttctgtgaatgcaaacacctcctgagttttgcaattgagaaaatgatttggagaacttctcttctggtaatttttattttgaatgttcagggccttagttggccccagtaattctccttggaggacttgggagaagaatttccacaaagcaaactactaaccactagctcttactggacagcgatttctggcttataagagttctctttgatttgcactagcactacgatagtgttagatggggaaatactgcaacatgtccagttggccagatcactttccaagggagcgatactaaggcagactcagctttttaaagatgggaggtcaggaggtggaagtgagaggagatcccatctcacacaacacacttccacgtaatgcagaccacacttttccattttgtcctgccctcttgagaggtcatttctcacgtcctaagaacctgatcagaaattttggaagggttctttgaaatagcagcagttgaaacagagacactttgccacagtgtggagcagattttctcactggtatcacatggtcttgcagttttgaactcttcgaccgatttgtgggagtttatgtaattgcgtgcaatgaacctgaaattgtgtaaaggacaaaagaccagtttatagggttgggttttttttccaacttgtgaaaagcagtttagctgcatctgtctccccaccacccccaccccgggaggggcttatgttacaaggtgatcaagtgaaggaaaaacctgagcctatctggctgggatggtggaattaagcacaaggtcacattctctgtgatcacatgagagggaaggtgatgacttaaatggcagggggtggggattatcttggggagaggctgaaaagcacaaaagatagtcttccctgtacgtattggtgaagaacgtgcacaaggctggatggacttcaacttggagttgagttgaggcaagaggatttctggatattagtcacccatctgcaagaaaaatgctgaggcctcgggtcaagattttgatctgagacatgctgatgcttcaaggagaaatattttcacaatcctctcttccctcaccagaagagaacagtactctctcctagaaacctctaggtaaacacattttatcctaatatcggtagcatataatgccccccccaaaatatctgttttccatgcaaaaaagtctcaacaagaagtctgtggagttgagtggttacttcaaagtgtcaggagagtgaagaaattggccacagaagagcaagaagctctcttaagaaaagggaattctctttaaagaaaccaccaccaacaacaaaacaaccaaaaaccatgttttatgtcaaagctctgtagcacagagaatgtggtgtcacagatacatcgccgagagaggtttctttctttcttttttttttttttgagacagagtctggttctgtttcccaggctggagtgcagtggtgggatctcagctcactgcaacatccgcctctggggttcaagtgattctcctgtctcagcctcccaagtagctggaattacagggacccgccaccacgcccggctaatttttttgtgtggttttagtagaggtggggtttcaccatcttggccaggctggtcttgaactcctgacctcgtgatccacccgcctaggcctcccaaagtgttgggattacaggcgtgagccactgtgcccagccaaaagagaaatttctacatgaacaaggcaatttcagtgtcttacagcggccaaaccatgacgtgaagaatgagataggagacaggagatcaccataagcgtccctgatatagcagcacacattttcacgtttccacttaaatcgttttgcacaaagtcttgcttcgctcagatgagatgagatatgatttcctagagatgtaaaaataagaatgaatgtggcgcccccttcttccagatgtaatagaaagctctgccctatcacaaggggggtgttgaagcgccccttgtgttttaactgtatttaactgagcacaagatgcacaagctgtggtgggaaaccctcagtttacctttggagtcttccctgcagatcgcagacctgtttccaggctgatgtttctggtgtgtaattgctagcgtttctgaagggttttcccaattgttttagccttgtgaagtattcttaattataacttgcctttcagcgatggtacatgacttgattcaacgtttggttctgaacttacacactgatgcgtttactcatctaacataatctgacagggcctcagcaagggagccatacatttttgtaacattttgatatgttttaatgcatctgacttagatcttactgaaataaagcacttttcaaagagaaaaaaaaaaaaaaaaaaaaaaa

SEQ ID NO: 5 - DNA, 954 bp, Homo sapiens
gcggagccgtgcggcggggcggggccgggcggcggggcctggtttcctccctcagcgccattttgtggcagcgagacccacaaataaaggggagcgcaggggttgcggcgggactaggagcgcggcggggccggcggcagagctgtccggctgcgcggtggcccggggggcccgggcggcagggcaagcagcgcggcctcggcctatgcgaccggtggcgccggcgcggcttctgcctggagaggtaggcgcgggccggctggcgggagcggacgcgggggacctccggggcctgagggctgatgcgcagccgcccccggcgggtaaggggcgggcagggcccgaaaggccacacgggcctggtgcggcaccggtctctcgtgagccgctctaccttcccgcctgcggggaacacttccgccgcttcgaggccattttatctctgtcctccgtccccaaggcccgggttgagaggggtgctctggcggccggagagaccggccactcacggaggccggaggatgccctccccgggctgagcggcggcgctcgttttttcctcgcctcggggtcgcccccttcacctcccttgggcctctcctgaggacctggcggcactaacgagttcagcaaggaaaaaaagaaacattcgttccctacatttcatgtcacgcagtaggatgttactacgttctcttgtagacttccatttttaaatgagtataagcattctgaggaaagaaactgaattattgcagatttttgtgaaatccaagtcttaccttggatccctgtgagacagttactagatttttttttcaccgtttttcataagtctgtgtacacgtaatggaaaagcccgagttcttgttttgataagaaagtcacttggttgtttgatcccaaaggattagcttaacattatcaaggactagcataactgtgattatgtaa

SEQ ID NO: 6 - DNA, 2382 bp, Homo sapiens
aagagggaagtcgtggtggtcgcgagggagccggaaagatggtggttaccagatctgcacgggctaaggccagcatccaagccgcgtcggctgaaagttccgggcaaaagagttttgctgctaatgggattcaagcgcatccagaaagtagtactggatctgatgcccgaactactgctgaatcacagaccactgggaagcaaagtttaatccctagaactcctaaagctagaaagaggaagagcagaactacaggctcactaccaaaggggactgaaccatctacggatggagagacctctgaggcagagtcaaattattctgtgtctgagcaccatgataccattttaagggtaactaggagaaggcagatcttaattgcatgctccccagtgtccagtgttaggaaaaagccgaaagtaactccaacaaaggagtcttacactgaagaaatagtgtctgaagcagaatctcatgtttcaggtatttctagaattgtgcttcctacagaaaaaactacaggagccagaagaagtaaggctaaatctctgacagatccaagccaagaatctcatacagaagctatatctgatgctgagacatcaagctcagacatttcattctctggaattgcaactagaagaaccaggagtatgcagaggaaattaaaggcacaaactgaaaagaaagatagtaagattgtaccaggaaatgagaaacagatcgtgggtacacctgtgaattcagaggattcagataccagacaaacttcccatttacaagcaagatctctttctgagataaataagccaaatttctataataatgactttgatgatgatttctcccacagaagttcagaaaatatattaacagtgcacgaacaggccaatgttgaatctcttaaagaaacaaaacagaattgtaaggatttggatgaagatgccaatggaataacagatgaggggaaagaaattaatgagaaaagttctcagctgaagaatctttctgaacttcaggacactagccttcaacagttagtttctcagagacattcaaccccccaaaataaaaatgctgtatcagtgcactctaatctgaactctgaggctgtaatgaaatcattaactcaaacatttgcaactgtggaagtaggcagatggaataacaacaaaaagagccccataaaagcaagtgacttgacaaagtttggtgattgtggtggtagtgatgatgaagaagagtccacagttataagtgtcagtgaagacatgaacagtgaagggaatgtagattttgaatgtgataccaaactatacacgtctgcgcccaacacatctcagggtaaagataattctgtcttactagttctcagcagtgatgaaagccaacagtctgaaaacagtgagaatgaagaggatactttatgttttgttgaaaatagtggccaaagggagtcattaagtggagacacaggaagtctgtcatgtgacaatgcattgtttgtaattgacacaactcctggaatgagtgctgataaaaatttttacttggaagaggaagacaaggcaagtgaggttgccattgaggaagaaaaagaagaggaagaggatgaaaaaagtgaagaagattcatcagaccatgacgaaaatgaagatgagtttagtgatgaagaagacttcctaaatagcacaaaggctaaacttctgaagttgacaagcagcagcatagaccctggtctgagtatcaagcagttgggtggtttgtatattaattttaatgcagataaactacagtctaacaagagaaccctaacacagatcaaggagaaaaagaaaaatgagcttctgcagaaagccgtcattacacctgattttgaaaaaaaccactgtgttccaccatatagtgaatcaaagtatcaacttcagaaaaaacgcagaaaagaacgacaaaaaacagcaggggatggctggtttggtatgaaagctccagaaatgacaaatgaactgaaaaatgatctcaaagcactgaagatgagagccagcatggacccgaaaagattttacaagaaaaatgatagagatggcttccccaagtacttccagattggaaccattgttgacaatccagctgatttctaccattcacgaattcccaagaagcaaaggaaaagaactattgtggaagaactgctggctgattctgaattcagaagatacaaccgaaggaagtactcagagatcatggctgaaaaagcagcaaatgcagcaggaaaaaagttccgaaagaagaagaaatttcgcaattaagatttaccaagcaaactgcaacattttacattgctcctttatttacttattaaagacgtttggaaaactaaaa

SEQ ID NO: 7 - DNA, 2512 bp, Homo sapiens
agcgggatcttgtcgtggcagctcatccaagcatttagctagaccctgtgattgcccgtggctctctgagtctgtcttattgagtagttagcagtattttttcctaaaattcagaagtcatctttgttacacaacacaggggttcaggtagcaataggacacaaaattgctttattctacaactgccagctccaggcagaaataggaaggcaaagagataagagaaggaaaaatgagagaatgaagtctgtatagggtagagcaatagaaagtaagcttcgggtgcctccaacgttcatggctgcctgtctcattggtaaacctcacatttagttacttgtggctactgcccacacatacacttctgtaattgagaactcttaggagaggactagggaatcactggggatagtgggctggagagaaccccaggctttatatgtatactttgacctcagtgttaattttaaatgcttatgaatcacacacattgctttagtaagattaagtgcttatatactagaaatttgatgctcattggaacacatctgcctagcatttctgtaaaagtcttaagtgatattaagatgattccttaccatttcagatggtccgcaatttgaattaccaagtggtaatggttccttactgttttagatggtgcctgtgagataccattcctctggatggtcatgtccagtcagtgggaggtagaaagggtggcatctgtagccctcttcatacacataagtggcatttaggtgaatgtcccagctaatcactagcatgtctgggtattggctgggtagtgggtattttgatgatctgggagcaccaaatatgttcattcttcgtttggggaggctggtctgtaaacacaaaaattgttgtccagatctttcatctgtttatgatcatcaacaaagacttgttagaaaggtctagtcttagcacttggcagttaatctagggaagatgaattaaatgggtagatagtgatgcatacctgtattcactgatgtatgtttaagggatttgggggggatacctcagttcatgtggaagggacagtctcggtgtgtcccatgaataaccttggaactgcaacaaatggtttgtgctcagaaaaagtctttcatggtgacaggaagacagtttccctggagctggccatgaaggccttagaagccatttctggtgtctggtgggtagcaggcatagagatgatgtgccgaggtcccagtgaacaacagtagccaaagaatgtactaactttatcattaataggaaagtcatacctaggaaacaatgactttttgatggcaaaatgattttttaattctattttgatgctgtaattccatttcatgacctagttgtattagaaaaccttgatgaactatatgttcccgatttacaaaaaaattaataaaacctccagagtaaactcagtcaaacaataattgagtagcagcttttataacatttaaaatttgcacatgagtgtgttgtcatatggagtgtctgaatctgttgctgggacataccaatccatgtattcattagagccatagaagttattattcattagttcatagtgtttgagttctttatgtcactctgttagaaacaaggactgagtcgtgaagaaataagaattggatttttataaaaacctctgaaggatatttacctatgaaaaagttgttaagaataaaaattagaagtccatggttaacttttccttcaatttatattattcctgaatcatagggaatctttctagaatgtgtttataatttccttgtacagtttctttggaaatacgttaaagatagtggcaatttcatatatttcatggatacttgagtttgtgcttttaaggtgtttgtttagggatacaatgaccactagatgtcgctgtttatccagtagactaagattgagtgttctttttgttcagcaactcttctaaaatgtttcaagcaaagatagtaatgacctcagtttctgaaataagggcatcttcatcagattattcttctgttttaaaaaaaagcttgaggcaaatgtgagtgatttccagtgctttgaaagggattacagtatcacacaatgtcaagctagagttaaacacagtattagctaaataggcacttatgtgtattttctttttcatgattatcggtgactggtcagtgtactcatcaatttccaaaatttgtataaatatcacaattagaaaaatgcctgaggtactaaagttatgttggctttttgtgtcttaacacacataaatactattgttattgcagcagatgccttttgaatccattttccataattgcggatagtcataaattgcttgctcaatttttagtaattattgctgttgacaccagcgttgtagatttttggtgttgttgaatgcagtagagagaccaagacactattctgtaagatcaataaaagtaattggaaaataaatatgaacccaaaaaaaaaaaaaaaa

SEQ ID NO: 8 - DNA, 3806 bp, Homo sapiens
cttcttgtaagagagtgctaggcacatagtcccctcctattcctaatcctcccaccaaagaaagaggcacagagttcattacttagtgggggccagctgtgatcggccaactgccagctgccttaaaaaggaagaccagtgatgctaggatggagtgaaacccaagaggaagtgccatcatgaggaatcaatgagagatctgtgaagagagagggctgggtgggagcccagaaggatagaacctggaagatcaatatctcccgtgagggaaataacaatggagccaggttctaagtcagtgtctaggtcagactggcaacctgaaccacaccagaggcctataaccccgctagagcctgggccagaaaagacacccatagcccagccagaatcgaagactctgcagggatccaatacccaacagaagcctgcttcaaaccaaagacccctcacccagcaggagacccctgcacaacatgatgctgaatcccagaaggaacctagagcccaacaaaaatctgcttcacaagaggaatttcttgccccacagaagcccgcaccacagcaatcaccttacatccaaagggtgctgctcactcaacaggaagctgcctcccagcagggacctgggctaggaaaagaatctataactcaacaggagccagcattgagacaaagacatgtagcccagccagggcctgggccaggagagccacctccagctcaacaagaagctgaatcaacacctgcggcccaggctaaacctggagccaaaagggagccatctgccccgactgaatctacgtcccaagagacacctgaacagtcagacaagcaaacaacgccagtccagggagccaaatccaagcagggatctttgacagagctgggatttctaacaaaacttcaggaactatccatacagcgatcagccctagagtggaaggcactttctgagtgggtcacagattctgagtcagaatcagatgtgggatcatcttcagacacagattctccagccacgatgggtggaatggtggcccagggagtgaagctaggcttcaaaggaaaatctggttataaagtgatgtcaggatacagtgggacgtcgccacatgagaaaaccagtgctcggaatcacagacactaccaggatacagcctcaaggctcatccacaacatggacctgcgcacaatgacacagtcgctggtgactctggcggaggacaacatagccttcttctcgagccagggtcctggggaaacggcccagcggctgtcaggcgtttttgccggtgtacgggagcaggcgctggggctggagccggccctgggccgcctgctgggtgtggcgcacctctttgacctggacccagagacaccggccaacgggtaccgcagcctagtgcacacagcccgctgctgcctggcgcacctcctgcacaaatcccgctatgtggcctccaaccgccgcagcatcttcttccgcaccagccacaacctggccgagctggaggcctacctggctgccctcacccagctccgcgctctggtctactacgcccagcgcctgctggttaccaatcggccgggggtactcttctttgagggcgacgaggggctcaccgccgacttcctccgggagtatgtcacgctgcataagggatgcttctatggccgctgcctgggcttccagttcacgcctgccatccggccattcctgcagaccatctccattgggctggtgtccttcggggagcactacaaacgcaacgagacaggcctcagtgtggccgccagctctctcttcaccagcggccgctttgccatcgaccccgagctgcgtggggctgagtttgagcggatcacacagaacctggacgtgcacttctggaaagccttctggaacatcaccgagatggaagtgctatcgtctctggccaacatggcatcggccaccgtgagggtaagccgcctgctcagcctgccacccgaagcctttgagatgccactgactgccgaccccacgctcacggtcaccatctcacccccactggcccacacaggccctgggcccgtcctcgtcaggctcatctcctatgacctgcgtgaaggacaggacagtgaggagctcagcagcctgataaagtccaacggccaacggagcctggagctgtggccgcgcccccagcaggcaccccgctcgcggtccctgatagtgcacttccacggcggtggctttgtggcccagacctccagatcccacgagccctacctcaagagctgggcccaggagctgggcgcccccatcatctccatcgactactccctggcccctgaggcccccttcccccgtgcgctggaggagtgcttcttcgcctactgctgggccatcaagcactgcgccctccttggctcaacaggggaacgaatctgccttgcgggggacagtgcaggcgggaacctctgcttcaccgtggctcttcgggcagcagcctacggggtgcgggtgccagatggcatcatggcagcctacccggccacaatgctgcagcctgccgcctctccctcccgcctgctgagcctcatggaccccttgctgcccctcagtgtgctctccaagtgtgtcagcgcctatgctggtgcaaagacggaggaccactccaactcagaccagaaagccctcggcatgatggggctggtgcggcgggacacagccctgctcctccgagacttccgcctgggtgcctcctcatggctcaactccttcctggagttaagtgggcgcaagtcccagaagatgtcggagcccatagcagagccgatgcgccgcagtgtgtctgaagcagcactggcccagccccagggcccactgggcacggattccctcaagaacctgaccctgagggacttgagcctgaggggaaactccgagacgtcgtcggacacccccgagatgtcgctgtcagctgagacacttagcccctccacaccctccgatgtcaacttcttattaccacctgaggatgcaggggaagaggctgaggccaaaaatgagctgagccccatggacagaggcctgggcgtccgtgccgccttccccgagggtttccacccccgacgctccagccagggtgccacacagatgcccctctactcctcacccatagtcaagaaccccttcatgtcgccgctgctggcacccgacagcatgctcaagagcctgccacctgtgcacatcgtggcgtgcgcgctggaccccatgctggacgactcggtcatgctcgcgcggcgactgcgcaacctgggccagccggtgacgctgcgcgtggtggaggacctgccgcacggcttcctgaccctagcggcgctgtgccgcgagacgcgccaggccgcagagctgtgcgtggagcgcatccgcctcgtcctcactcctcccgccggagccgggccgagcggggagacgggggctgcgggggtagacgggggctgcggggggcgacactaaaagcctgttgttcccatctgcgccggcctccgtcatgaatgccttccgggccgggcggaaggggacgcgggctgtgccttacttaagtcgggggtggcaagggggcggggcgggggcccgaaagctgagaccctcgccacggggagggggacgcgcacacacaccggtcaccgagacggctggacctgcacgccaccgctgccttttgctgctgctgctgcggcgaccgccgcagggacggggactggccctcccttgcaggtcggtttggtttgttgtaaataaaagtatttaatta

SEQ ID NO: 9 - DNA, 843 bp, Homo sapiens
gggcgggtcttcttccttctcgcctaacgccgccaacatggtgttcaggcgcttcgtggaggttggccgggtggcctatgtctcctttggacctcatgccggaaaattggtcgcgattgtagatgttattgatcagaacagggctttggtcgatggaccttgcactcaagtgaggagacaggccatgcctttcaagtgcatgcagctcactgatttcatcctcaagtttccgcacagtgcccaccagaagtatgtccgacaagcctggcagaaggcagacatcaatacaaaatgggcagccacacgatgggccaagaagattgaagccagagaaaggaaagccaagatgacagattttgatcgttttaaagttatgaaggcaaagaaaatgaggaacagaataatcaagaatgaagttaagaagcttcaaaaggcagctctcctgaaagcttctcccaaaaaagcacctggtactaagggtactgctgctgctgctgctgctgctgctgctgctgctaaagttccagcaaaaaagatcaccgccgcgagtaaaaaggctccagcccagaaggttcctgcccagaaagccacaggccagaaagcagcgcctgctccaaaagctcagaagggtcaaaaagctccagcccagaaagcacctgctccaaaggcatctggcaagaaagcataagtggcaatcataaaaagtaataaaggttctttttgacctgttgacaaatgtatttaagcctttggatttaaagcctgttgaggctagagttaggaggcagattgatagtaggattataataaacattaaataatcaaaaaaaaaaaaaaaaaaaa